(19) United States
(12) Patent Application Publication
(10) Pub. No.: US 2024/0117299 A1
(43) Pub. Date: Apr. 11, 2024

(54) STREPTOCOCCUS THERMOPHILUS STARTER STRAIN

(71) Applicant: DSM IP Assets B.V., HEERLEN (NL)

(72) Inventors: Lourdes Mariela SERRANO DAVALOS, ECHT (NL); Paul KLAASSEN, ECHT (NL); Ali MAY, ECHT (NL); Sofia DASHKO, ECHT (NL); Claire Emile PRICE, ECHT (NL); Paulus Petrus DE WAAL, ECHT (NL)

(21) Appl. No.: 18/484,980

(22) Filed: Oct. 11, 2023

(30) Foreign Application Priority Data
Oct. 11, 2022 (EP) 22200834.4

(51) Int. Cl. C12N 1/20; A23C 9/123

(52) U.S. Cl. CPC C12N 1/205; A23C 9/1238; C12R 2001/46

(57) ABSTRACT

The invention relates to the field of dairy technology. The invention specifically relates to a Streptococcus thermophilus starter strain and composition comprising such strain, to methods to produce a fermented milk product using such strain or composition and to dairy products produced using such strain or composition.

### CROSS-REFERENCE TO RELATED APPLICATIONS

This application claims priority to European Patent Application No. 22200834.4, filed 11 Oct. 2022, the entire contents of which are incorporated herein by reference.

### REFERENCE TO SEQUENCE LISTING SUBMITTED AS A COMPLIANT ASCII TEXT FILE (.txt)/(.xml)

Pursuant to the EFS-Web legal framework and 37 CFR §§ 1.821-825 (see MPEP § 2442.03(a)), a Sequence Listing in eXtensible Markup Language (XML) format (named “Sequence_Listing_2919208-621000” created on 11 Oct. 2023 and having a size of 324,931 bytes) is submitted concurrently herewith. The material in the aforementioned file is hereby incorporated by reference in its entirety.

### BACKGROUND

### Field

The invention relates to the field of dairy technology. The invention specifically relates to a Streptococcus thermophilus starter strain and composition comprising such strain, to methods to produce a fermented milk product using such strain or composition and to dairy products produced using such strain or composition.

### Description of Related Art

Lactic acid bacteria are widely used in the food industry to improve taste and texture of food products as well as to extend the shelf life of food products. In the dairy industry, lactic acid bacteria are used to acidify milk and to texturize the product by fermentation. Widely used lactic acid bacteria are species of Streptococcus, Lactococcus, Lactobacillus, Leuconostoc and Bifidobacterium. Streptococcus thermophilus (S. thermophilus) is used extensively either alone or in combination with other lactic acid bacteria such as Lactobacillus delbrueckii subspecies bulgaricus (L. bulgaricus). These bacteria are widely used for the production of fermented milk products such as yoghurts. During fermentation of milk wherein milk is transformed into yoghurt, the product is acidified and texture is developed by secretion of polysaccharides (exopolysaccharides or EPS) by the lactic acid bacteria.

Within consumers of fermented milk products, such as yoghurts, there is a trend for increased demand of products with mild flavour and thus be attractive to be able to produce yoghurts of the desired texture without having to add artificial agents; such would result in a more natural product and reduction of production costs. A further problem with state of the art yoghurt production is that after acidification of the product, during shelf life, post-acidification occurs; i.e. the pH of the food product is further lowered by the lactic acid bacteria present in the yoghurt. It would thus be attractive to be able to produce yoghurts with desired texture while reducing post-acidification.

Whilst a mild flavour and low post-acidification are desired from a consumer perspective, producers still desire a short acidification time. As a result there is a desire to have a fast acidification of a milk base to a fermented milk product, followed by low post-acidification in such fermented milk base once a certain pH has been reached.

It would be a further advantage in the art to provide lactic acid bacteria, especially Streptococcus thermophilus bacteria, that allows for a good viability of the strain, short acidification time, combined with low post-acidification.

### BRIEF SUMMARY

Inventors have now advantageously found special Streptococcus thermophilus bacteria, that allow for a short acidification time, combined with low post-acidification and acceptable or even improved viability of the strain.

The invention thus provides in a first aspect an isolated Streptococcus thermophilus strain, wherein the activity of a native ABC transporter complex ATP binding protein or a component thereof, is reduced, impaired, or disabled.

This isolated Streptococcus thermophilus strain may or may not be the Streptococcus thermophilus strain deposited as CBS149207 at the Westerdijk Fungal Biodiversity Institute, Uppsalalaan 8, 3548 CT, the Netherlands on 14 Jun. 2022. In addition, the isolated Streptococcus thermophilus strain may or may not be the Streptococcus thermophilus strain deposited as CBS149207 at the Westerdijk Fungal Biodiversity Institute, Uppsalalaan 8, 3548 CT, the Netherlands on 14 Jun. 2022.

The invention also provides in a second aspect an isolated Streptococcus thermophilus strain, comprising, as compared to wild-type strain Streptococcus thermophilus ASCC1275:
a mutation in the nucleotide sequence of SEQ ID NO: 1;
and
optionally a mutation in the nucleotide sequence of SEQ ID NO: 19; and/or
optionally a mutation in the nucleotide sequence of SEQ ID NO: 35; and/or
optionally a mutation in the nucleotide sequence of SEQ ID NO: 43; and/or
optionally a mutation in the nucleotide sequence of SEQ ID NO: 57; and/or
optionally a mutation in the nucleotide sequence of SEQ ID NO: 83.

The invention also provides in a third aspect an isolated Streptococcus thermophilus strain, comprising, as compared to wild-type strain Streptococcus thermophilus strain MN-ZLW-002:
a mutation in the nucleotide sequence of SEQ ID NO: 89;
and
optionally a mutation in the nucleotide sequence of SEQ ID NO: 123; and/or
optionally a mutation in the nucleotide sequence of SEQ ID NO: 125; and/or
optionally a mutation in the nucleotide sequence of SEQ ID NO: 129; and/or
optionally a mutation in the nucleotide sequence of SEQ ID NO: 141; and/or
optionally a mutation in the nucleotide sequence of SEQ ID NO: 143; and/or
optionally a mutation in the nucleotide sequence of SEQ ID NO: 151.

Without wishing to be bound by any kind of theory, it is believed that ABC transporter complex ATP binding proteins may play a role in the mechanisms on how bacteria handle stressful situations, such as an acid environment.

Provided is further an isolated Streptococcus thermophilus strain CBS149207 and mutants and variants thereof or an isolated Streptococcus thermophilus strain CBS149208 and mutants and variants thereof, wherein said mutants and variants thereof preferably comprise at least one mutation selected from the groups of mutations consisting of F1 to F43 as depicted in Table 1 and C1 to C35 as depicted in Table 2.

Further provided is a method for the production of an isolated Streptococcus thermophilus strain comprising:
a. providing a parent Streptococcus thermophilus strain,
b. mutagenizing the Streptococcus thermophilus strain,
c. selecting for a mutant with improved milk acidification characteristics, preferably at least one of the milk acidification characteristics, and
d. isolating the mutant with the improved milk acidification characteristics.

Further provided is an isolated Streptococcus thermophilus strain obtainable by said method.

Further provided is a starter culture for the production of a fermented milk product preferably having elongated shelf life and/or preferably having the ability to be stored at elevated temperature, comprising an isolated Streptococcus thermophilus strain according to the invention.

Further provided is the use of the isolated Streptococcus thermophilus strain according to the invention or of the starter culture according to the invention for the production of a fermented milk product.

Further provided is a fermented milk product comprising a Streptococcus thermophilus strain according to the invention.

### BRIEF DESCRIPTION OF THE DRAWINGS

The invention is illustrated by the following figures:

FIG. 1: Acidification curves (in duplicate) of CBS149207 and the parent strain of CBS149207.

FIG. 2: Acidification curves (in duplicate) of CBS149208 and the parent strain of CBS149208.

### BRIEF DESCRIPTION OF THE SEQUENCE LISTING

This application contains a Sequence Listing in computer readable form, which is incorporated herein by reference. An overview of the sequences is provided by Tables 1, 2 and 3 below.

### DETAILED DESCRIPTION

The isolated Streptococcus thermophilus strain

The invention provides in a first aspect an isolated Streptococcus thermophilus strain, wherein the activity of a ABC transporter complex ATP binding protein or a component thereof, is reduced, impaired, or disabled. The ABC transporter complex ATP binding protein is suitably native to the Streptococcus thermophilus strain. As will be understood by the person skilled in the art, the invention therefore suitably provides an isolated Streptococcus thermophilus strain, wherein the activity of a native ABC transporter complex ATP binding protein or a component thereof, can suitably be reduced, impaired, or disabled compared to its parent strain and/or a wild-type strain.

The term “ABC transporter complex ATP binding protein” is herein preferably understood to refer to an ATP binding protein in an ABC transporter complex. The term “ABC transporter complex” is herein preferably understood to refer to the complex of ATP-binding cassette transporters (also referred to as “ABC transporters”) that form the transport system that arranges the transport of compounds across the cellular membrane in the bacteria.

A subcategory of the ABC transporters is formed by ATPase. The term “ATPase” is herein preferably understood to refer to a protein that catalyses the decomposition of Adenosine triphosphate (ATP) to Adenosine diphosphate (ADP).

In this invention, the ABC transporter complex ATP binding protein is preferably an ATPase, more preferably a cell-membrane associated and/or cell-membrane bound, ATPase. Without wishing to be bound by any kind of theory, it is believed that the ATPase utilize the energy of adenosine triphosphate (ATP) binding and hydrolysis to provide the energy needed for the transport of compounds across membranes, either for uptake or for export of such compound.

Preferably the ABC transporter complex ATP binding protein is an ATPase, more preferably a cell-membrane associated and/or cell-membrane bound ATPase. That is, in such case the invention provides an isolated Streptococcus thermophilus strain, wherein the activity of a native ATPase, preferably a native cell-membrane associated and/or cell-membrane bound ATPase, is reduced, impaired, or disabled. Again, as will be understood by the person skilled in the art, such reduction, impairment, or disablement will preferably be as compared to its parent strain and/or the wild-type strain from which the isolated Streptococcus thermophilus strain can be derived.

The ATP binding protein can be an import ATP-binding protein or an export ATP binding protein. Preferably the ATP binding protein is an import ATP binding protein.

In one preferred embodiment the ABC transporter complex ATP binding protein is a hemin import ATP-binding protein. More preferably the ABC transporter complex ATP binding protein is a HrtA protein and/or a HrtA_2 protein.

In another preferred embodiment the ABC transporter complex ATP binding protein is a vitamin B12 import ATP-binding protein. In this case, more preferably the ABC transporter complex ATP binding protein is a BtuD protein.

The activity of the ABC transporter complex ATP binding protein can be reduced in several different ways. In this invention preferably the ABC transporter complex ATP binding protein is mutated causing it to have a reduced, impaired or disabled activity and/or to be otherwise reduced, impaired or disabled in its function. Such a mutation is preferably achieved by a mutation in the genes encoding for the ABC transporter complex ATP binding protein.

Hence, the invention also provides an isolated Streptococcus thermophilus strain, wherein a gene encoding for a ABC transporter complex ATP binding protein is mutated such that the activity of the ABC transporter complex ATP binding protein or a component thereof, is reduced, impaired, or disabled. The gene encoding for the ABC transporter complex ATP binding protein is suitably native to the Streptococcus thermophilus strain. Hence, the invention suitably provides an isolated Streptococcus thermophilus strain, wherein a native gene encoding for a ABC transporter complex ATP binding protein is mutated such that the activity of the ABC transporter complex ATP binding protein or a component thereof, is reduced, impaired, or disabled. Again, as will be understood by the person skilled in the art, such reduction, impairment, or disablement will preferably be as compared to its parent strain and/or the wild-type strain from which the isolated Streptococcus thermophilus strain can be derived.

In one preferred embodiment this invention provides an isolated Streptococcus thermophilus strain, comprising, as preferably compared to wild-type strain Streptococcus thermophilus ASCC1275,
a mutation in the HrtA_2 protein; and
optionally a mutation in the ALDC protein; and/or
optionally a mutation in the PURL protein; and/or
optionally a mutation in the RNHB protein; and/or
optionally a mutation in the DNAE2 protein; and/or
optionally a mutation in the STHA protein,
wherein preferably the activity of the HrtA_2 protein, and optionally one or more of the other proteins, or a component thereof, is reduced, impaired, or disabled, as preferably compared to wild-type strain Streptococcus thermophilus ASCC1275.

More preferably this invention provides an isolated Streptococcus thermophilus strain, comprising, as preferably compared to wild-type strain Streptococcus thermophilus ASCC1275,
a mutation in the hrtA_2 gene; and
optionally a mutation in the aldC gene; and/or
optionally a mutation in the purI gene; and/or
optionally a mutation in the rnhB gene; and/or
optionally a mutation in the dnaE2 gene; and/or
optionally a mutation in the sthA gene.

More preferably this invention provides an isolated Streptococcus thermophilus strain, comprising:
one or more genes encoding for an amino acid sequence that has equal to or more than 90%, more preferably equal to or more than 95%, and most preferably equal to or more than 99% sequence identity with the amino acid sequence of SEQ NO: 2 and comprises at least one mutation compared to the amino acid sequence of SEQ NO: 2; and
optionally one or more genes encoding for an amino acid sequence that has equal to or more than 90%, more preferably equal to or more than 95%, and most preferably equal to or more than 99% sequence identity with the amino acid sequence of SEQ NO: 20 and comprises at least one mutation compared to the amino acid sequence SEQ NO: 20; and/or
optionally one or more genes encoding for an amino acid sequence that has equal to or more than 90%, more preferably equal to or more than 95%, and most preferably equal to or more than 99% sequence identity with the amino acid sequence of SEQ NO: 36 and comprises at least one mutation compared to the amino acid sequence SEQ NO: 36; and/or
optionally one or more genes encoding for an amino acid sequence that has equal to or more than 90%, more preferably equal to or more than 95%, and most preferably equal to or more than 99% sequence identity with the amino acid sequence of SEQ NO: 44 and comprises at least one mutation compared to the amino acid sequence SEQ NO: 44; and/or
optionally one or more genes encoding for an amino acid sequence that has equal to or more than 90%, more preferably equal to or more than 95%, and most preferably equal to or more than 99% sequence identity with the amino acid sequence of SEQ NO: 58 and comprises at least one mutation compared to the amino acid sequence SEQ NO: 58; and/or
optionally one or more genes encoding for an amino acid sequence that has equal to or more than 90%, more preferably equal to or more than 95%, and most preferably equal to or more than 99% sequence identity with the amino acid sequence of SEQ NO: 84 and comprises at least one mutation compared to the amino acid sequence SEQ NO: 84.

Most preferably this invention provides an isolated Streptococcus thermophilus strain, comprising:
a nucleotide sequence of SEQ NO: 157 and/or one or more genes encoding for a protein comprising or consisting of an amino acid sequence of SEQ NO: 158; and
optionally a nucleotide sequence of SEQ NO: 159 and/or one or more genes encoding for a protein comprising or consisting of an amino acid sequence of SEQ NO: 160;
and/or
optionally a nucleotide sequence of SEQ NO: 161 and/or one or more genes encoding for a protein comprising or consisting of an amino acid sequence of SEQ NO: 162;
and/or
optionally a nucleotide sequence of SEQ NO: 163 and/or one or more genes encoding for a protein comprising or consisting of an amino acid sequence of SEQ NO: 164;
and/or
optionally a nucleotide sequence of SEQ NO: 165 and/or one or more genes encoding for a protein comprising or consisting of an amino acid sequence of SEQ NO: 166;
and/or
optionally a nucleotide sequence of SEQ NO: 167 and/or one or more genes encoding for a protein comprising or consisting of an amino acid sequence of SEQ NO: 168.

In another preferred embodiment this invention provides an isolated Streptococcus thermophilus strain, comprising, as preferably compared to wild-type strain Streptococcus thermophilus MN-ZLW-002,
a mutation in the HRTA protein; and
optionally a mutation in the PYRK protein; and/or
optionally a mutation in the OPPD_4 protein; and/or
optionally a mutation in the ARGD protein; and/or
optionally a mutation in the BTUD_1 protein; and/or
optionally a mutation in the SCMP protein, and/or
optionally a mutation in the MPRF protein.
wherein preferably the activity of the HRTA protein, and optionally one or more of the other proteins, or a component thereof, is reduced, impaired, or disabled, as preferably compared to wild-type strain Streptococcus thermophilus MN-ZLW-002.

More preferably this invention provides an isolated Streptococcus thermophilus strain, comprising, as preferably compared to wild-type strain Streptococcus thermophilus MN-ZLW-002,
a mutation in the hrtA gene; and
optionally a mutation in the pyrK gene; and/or
optionally a mutation in the oppD_4 gene; and/or
optionally a mutation in the argD gene; and/or
optionally a mutation in the btuD_1 gene; and/or
optionally a mutation in the scmP gene, and/or
optionally a mutation in the mprF gene.

A combination of mutations in the hrtA gene and the btuD_1 gene is especially preferred.

More preferably this invention provides an isolated Streptococcus thermophilus strain, comprising:
one or more genes encoding for an amino acid sequence that has equal to or more than 90%, more preferably equal to or more than 95%, and most preferably equal to or more than 99% sequence identity with the amino acid sequence of SEQ NO: 90 and comprises at least one mutation compared to the amino acid sequence of SEQ NO: 90; and
optionally one or more genes encoding for an amino acid sequence that has equal to or more than 90%, more preferably equal to or more than 95%, and most preferably equal to or more than 99% sequence identity with the amino acid sequence of SEQ NO: 124 and comprises at least one mutation compared to the amino acid sequence SEQ NO: 124; and/or
optionally one or more genes encoding for an amino acid sequence that has equal to or more than 90%, more preferably equal to or more than 95%, and most preferably equal to or more than 99% sequence identity with the amino acid sequence of SEQ NO: 126 and comprises at least one mutation compared to the amino acid sequence SEQ NO: 126; and/or
optionally one or more genes encoding for an amino acid sequence that has equal to or more than 90%, more preferably equal to or more than 95%, and most preferably equal to or more than 99% sequence identity with the amino acid sequence of SEQ NO: 130 and comprises at least one mutation compared to the amino acid sequence SEQ NO: 130; and/or
optionally one or more genes encoding for an amino acid sequence that has equal to or more than 90%, more preferably equal to or more than 95%, and most preferably equal to or more than 99% sequence identity with the amino acid sequence of SEQ NO: 142 and comprises at least one mutation compared to the amino acid sequence SEQ NO: 142; and/or
optionally one or more genes encoding for an amino acid sequence that has equal to or more than 90%, more preferably equal to or more than 95%, and most preferably equal to or more than 99% sequence identity with the amino acid sequence of SEQ NO: 144 and comprises at least one mutation compared to the amino acid sequence SEQ NO: 144; and/or.
optionally one or more genes encoding for an amino acid sequence that has equal to or more than 90%, more preferably equal to or more than 95%, and most preferably equal to or more than 99% sequence identity with the amino acid sequence of SEQ NO: 152 and comprises at least one mutation compared to the amino acid sequence SEQ NO: 152.

Most preferably this invention provides an isolated Streptococcus thermophilus strain, comprising:
a nucleotide sequence of SEQ NO: 169 and/or one or more genes encoding for a protein comprising or consisting of an amino acid sequence of SEQ NO: 170; and
optionally a nucleotide sequence of SEQ NO: 171 and/or one or more genes encoding for a protein comprising or consisting of an amino acid sequence of SEQ NO: 172;
and/or
optionally a nucleotide sequence of SEQ NO: 173 and/or one or more genes encoding for a protein comprising or consisting of an amino acid sequence of SEQ NO: 174;
and/or
optionally a nucleotide sequence of SEQ NO: 175 and/or one or more genes encoding for a protein comprising or consisting of an amino acid sequence of SEQ NO: 176;
and/or
optionally a nucleotide sequence of SEQ NO: 177 and/or one or more genes encoding for a protein comprising or consisting of an amino acid sequence of SEQ NO: 178;
and/or
optionally a nucleotide sequence of SEQ NO: 179 and/or one or more genes encoding for a protein comprising or consisting of an amino acid sequence of SEQ NO: 180;
and/or
optionally a nucleotide sequence of SEQ NO: 181 and/or one or more genes encoding for a protein comprising or consisting of an amino acid sequence of SEQ NO: 182.

In addition, the invention provides an isolated Streptococcus thermophilus strain, comprising, as compared to wild-type strain Streptococcus thermophilus ASCC1275:
a mutation in the nucleotide sequence of SEQ ID NO: 1;
and
optionally a mutation in the nucleotide sequence of SEQ ID NO: 19; and/or
optionally a mutation in the nucleotide sequence of SEQ ID NO: 35; and/or
optionally a mutation in the nucleotide sequence of SEQ ID NO: 43; and/or
optionally a mutation in the nucleotide sequence of SEQ ID NO: 57; and/or
optionally a mutation in the nucleotide sequence of SEQ ID NO: 83.

Further the invention provides an isolated Streptococcus thermophilus strain, comprising, as compared to wild-type strain Streptococcus thermophilus ASCC1275,
a mutation in the hrtA_2 gene; and
optionally a mutation in the aldC gene; and/or
optionally a mutation in the purI gene; and/or
optionally a mutation in the rnhB gene; and/or
optionally a mutation in the dnaE2 gene; and/or
optionally a mutation in the sthA gene.

Still further the invention provides an isolated Streptococcus thermophilus strain, comprising, as compared to wild-type strain Streptococcus thermophilus ASCC1275
a c.511G>A mutation in the hrtA_2 gene; and
optionally a c.473G>A mutation in the aldC gene; and/or
optionally a c.988G>A mutation in the purI gene; and/or
optionally a c.605G>A mutation in the rnhB gene; and/or
optionally a c.613C>T mutation in the dnaE2 gene; and/or
optionally a c.203G>A mutation in the sthA gene.

Even still further the invention provides an isolated Streptococcus thermophilus strain, comprising, as compared to wild-type strain Streptococcus thermophilus strain MN-ZLW-002:
a mutation in the nucleotide sequence of SEQ ID NO: 89;
and
optionally a mutation in the nucleotide sequence of SEQ ID NO: 123; and/or
optionally a mutation in the nucleotide sequence of SEQ ID NO: 125; and/or
optionally a mutation in the nucleotide sequence of SEQ ID NO: 129; and/or
optionally a mutation in the nucleotide sequence of SEQ ID NO: 141; and/or
optionally a mutation in the nucleotide sequence of SEQ ID NO: 143; and/or
optionally a mutation in the nucleotide sequence of SEQ ID NO: 151.

The isolated Streptococcus thermophilus strain according to the invention may or may not be the Streptococcus thermophilus strain deposited as CBS149207 at the Westerdijk Fungal Biodiversity Institute, Uppsalalaan 8, 3548 CT, the Netherlands on 14 Jun. 2022. In addition, the isolated Streptococcus thermophilus strain may or may not be the Streptococcus thermophilus strain deposited as CBS149208 at the Westerdijk Fungal Biodiversity Institute, Uppsalalaan 8, 3548 CT, the Netherlands on 14 Jun. 2022.

More preferably the isolated Streptococcus thermophilus strain according to the invention is the isolated Streptococcus thermophilus strain deposited as CBS149207 at the Westerdijk Fungal Biodiversity Institute, Uppsalalaan 8, 3548 CT, the Netherlands on 14 Jun. 2022 or the isolated Streptococcus thermophilus strain may or may not be the Streptococcus thermophilus strain deposited as CBS149208 at the Westerdijk Fungal Biodiversity Institute, Uppsalalaan 8, 3548 CT, the Netherlands on 14 Jun. 2022.

Most preferably the isolated Streptococcus thermophilus strain according to the invention is the isolated Streptococcus thermophilus strain deposited as CBS149207 at the Westerdijk Fungal Biodiversity Institute, Uppsalalaan 8, 3548 CT, the Netherlands on 14 Jun. 2022.

In a first aspect, there is provided for an isolated Streptococcus thermophilus strain CBS149207 and mutants and variants thereof or an isolated Streptococcus thermophilus strain CBS149208 and mutants and variants thereof, wherein said mutants and variants thereof preferably comprise at least one mutation selected from the groups of mutations consisting of F1 to F43 as depicted in Table 1 and C1 to C35 as depicted in Table 2. In the embodiments herein, Streptococcus thermophilus strain CBS149207 and mutants and variants thereof and Streptococcus thermophilus strain CBS149208 and mutants and variants thereof may collectively be referred as the Streptococcus thermophilus strain according to the invention. In the embodiments herein, the genome of the mutant or variant of Streptococcus thermophilus strain CBS149207 preferably has at least 90%, 91%, 92%, 93%, 94%, 95%, 96%, 97%, 98%, at least 99% or 100% sequence identity with the genome of Streptococcus thermophilus strain CBS149207. In the embodiments herein, the genome of the mutant and variant of Streptococcus thermophilus strain CBS149208 preferably has at least 90%, 91%, 92%, 93%, 94%, 95%, 96%, 97%, 98%, at least 99% or 100% sequence identity with the genome of Streptococcus thermophilus strain CBS149208. In the embodiments herein, a mutant or variant of Streptococcus thermophilus strain CBS149207 preferably has at least one, 2, 3, 4, 5, 6, 7, 8, 9, 10, 11, 12, 13, 14, 15, 16, 17, 18, 19, 20, 21, 22, 23, 24, 25, 2, 6 2, 7, 28, 29, 30, 31, 32, 33, 34, 35, 36, 37, 38, 39, 40, 41, 42, or 43 mutations selected from the group depicted in Table 1. Preferred mutations are selected from F20 to F43, more preferred mutations are selected from F8 to F19 and most preferred mutations are selected from F1 to F7. The mutant or variant of Streptococcus thermophilus strain CBS149207 preferably has or further has at least one mutation selected from the group depicted in Table 2; preferred mutations are selected from C1 to C8. In the embodiments herein, a mutant or variant of Streptococcus thermophilus strain CBS149208 preferably has at least one, 2, 3, 4, 5, 6, 7, 8, 9, 10, 11, 12, 13, 14, 15, 16, 17, 18, 19, 20, 21, 22, 23, 24, 25, 2, 6 2, 7, 28, 29, 30, 31, 32, 33, 34, or 35 mutations selected from the group depicted in Table 2. Preferred mutations are selected from C21 to C35, more preferred mutations are selected from C9 to C20 and most preferred mutations are selected from C1 to C8. The mutant or variant of Streptococcus thermophilus strain CBS149208 has or further has at least one mutation selected from the group depicted in Table 1; preferred mutations are selected from F1 to F7.

In the embodiments herein, the mutants and variants preferably demonstrate the same or substantially the same milk acidification characteristics as Streptococcus thermophilus strain CBS149207 or CBS149208 in the preparation of a fermented milk product. In the embodiments herein, the milk acidification characteristics in the preparation of a fermented milk product preferably comprise acidification of a milk substrate to a pH of about or exactly 4.6 in less than 5 hours and a pH variation of less than 0.2 unit during storage of the freshly prepared fermented milk product at 25° Celsius for 28 days. Preferably, the time to reach a pH of 4.4 after a pH of 4.6 has been reached is at least 15%, or, more preferably, at least 20%, 25%, or at least 30% more than compared to the time required to reach a pH of 4.4 after a pH of 4.6 has been reached by Streptococcus thermophilus ASCC1275 or Streptococcus thermophilus MN-ZLW-002. In the embodiments herein, the isolated Streptococcus thermophilus strain preferably is not be able to consume galactose. In the embodiments herein, the milk acidification characteristics preferably further comprise viscosity of the fermented milk product of at least about or exactly 300 mPa·s, preferably the viscosity is within the range of about or exactly 300 mPa·s to about or exactly 1000 mPa·s. Herein less than 5 hours, is preferably less than 300 minutes, 290, 280, 270, 260, 250, or less than 240 minutes.

Further provided is an isolated Streptococcus thermophilus strain according to the invention having a genome with at least 80%, 81%, 82%, 83%, 84%, 85%, 86%, 87%, 88%, 89%, 90%, 91%, 92%, 93%, 94%, 95%, 96%, 97%, 98%, or at least 99% sequence identity to the genome of Streptococcus thermophilus ASCC1275 or Streptococcus thermophilus MN-ZLW-002, wherein the isolated Streptococcus thermophilus strain demonstrates the same or substantially the same milk acidification characteristics as Streptococcus thermophilus strain CBS149207 or CBS149208 in the preparation of a fermented milk product, wherein the milk acidification characteristics in the preparation of a fermented milk product comprise acidification of a milk substrate to a pH of about or exactly 4.6 in less than hours and a pH variation of less than 0.2 unit during storage of the freshly prepared milk product at 25° Celsius for 28 days, and wherein the Streptococcus thermophilus strain comprises at least one mutation selected from the groups of mutations consisting of F1 to F43 as depicted in Table 1 and C1 to C35 as depicted in Table 2. In the embodiments herein, a mutant or variant of Streptococcus thermophilus strain CBS149207 preferably has at least one, 2, 3, 4, 5, 6, 7, 8, 9, 10, 11, 12, 13, 14, 15, 16, 17, 18, 19, 20, 21, 22, 23, 24, 25, 2, 6 2, 7, 28, 29, 30, 31, 32, 33, 34, 35, 36, 37, 38, 39, 40, 41, 42, or 43 mutations selected from the group depicted in Table 1. Preferred mutations are selected from F20 to F43, more preferred mutations are selected from F8 to F19 and most preferred mutations are selected from F1 to F7. The mutant or variant of Streptococcus thermophilus strain CBS149207 preferably has or further has at least one mutation selected from the group depicted in Table 2; preferred mutations are selected from C1 to C8. In the embodiments herein, a mutant or variant of Streptococcus thermophilus strain CBS149208 preferably has at least one, 2, 3, 4, 5, 6, 7, 8, 9, 10, 11, 12, 13, 14, 15, 16, 17, 18, 19, 20, 21, 22, 23, 24, 25, 2, 6 2, 7, 28, 29, 30, 31, 32, 33, 34, or 35 mutations selected from the group depicted in Table 2. Preferred mutations are selected from C21 to C35, more preferred mutations are selected from C9 to C20 and most preferred mutations are selected from C1 to C8. The mutant or variant of Streptococcus thermophilus strain CBS149208 preferably has or further has at least one mutation selected from the group depicted in Table 1; preferred mutations are selected from F1 to F7. In the embodiments herein, the Streptococcus thermophilus strain according to the invention preferably is not be able to consume galactose. In the embodiments herein, the milk acidification characteristics further comprise viscosity of the fermented milk product of at least about or exactly 300 mPa·s, preferably the viscosity is within the range of about or exactly 300 mPa·s to about or exactly 1000 mPa·s.

In the embodiments herein, the mutant or variant of the Streptococcus thermophilus strain according to the invention may be obtained by mutagenesis or by genome editing and subsequent selection for the milk acidification characteristics as defined herein. The person skilled in the art knows adequate methods of mutagenesis and genome editing.

Further provided is a method for the production of an isolated Streptococcus thermophilus strain comprising:
providing a parent Streptococcus thermophilus strain,
mutagenizing the Streptococcus thermophilus strain,
selecting for a mutant with improved milk acidification characteristics, preferably at least one of the milk acidification characteristics as defined elsewhere herein, and isolating the mutant with the improved milk acidification characteristics. An improved milk acidification characteristic preferably is decreased post-acidification as compared to the parent strain. Preferably, the time to reach a pH of 4.4 after a pH of 4.6 has been reached is preferably at least 15%, or, more preferably, at least 20%, 25%, or at least 30% more than compared to the time required to reach a pH of 4.4 after a pH of 4.6 has been reached by the parent strain. Preferably, the improved milk acidification characteristic further comprises viscosity of the fermented milk product of at least about or exactly 300 mPa·s, preferably the viscosity is within the range of about or exactly 300 mPa·s to about or exactly 1000 mPa·s. Preferably, the parent Streptococcus thermophilus strain is ASCC1275 or is MN-ZLW-002, or wherein the parent strain is a Streptococcus thermophilus strain with substantially the same milk acidification characteristics as ASCC1275 or is MN-ZLW-002. Further provided is an isolated Streptococcus thermophilus strain obtainable by said method. Such strain is also referred to as a Streptococcus thermophilus strain according to the invention.

Further provided is a starter culture for the production of a fermented milk product preferably having elongated shelf life and/or preferably having the ability to be stored at elevated temperature, comprising an isolated Streptococcus thermophilus strain according to the invention. The starter culture may be a composition or a kit of parts. Elevated temperature is a temperature above 4° C., such as least 4, 5, 6, 7, 8, 9, 10, 11, 12, 13, 14, 15, 16, 17, 18, 19, 20, 21, 22, 23, 24 or at least 25° C. or 5, 6, 7, 8, 9, 10, 11, 12, 13, 14, 15, 16, 17, 18, 19, 20, 21, 22, 23, 24 or 25° C. Preferably, the fermented milk product has a pH of about or exactly 4.6 when freshly prepared and has a pH variation of less than 0.2 unit during storage of the freshly prepared milk product at 25° Celsius for 28 days. Preferably, the fermented milk product has a viscosity of at least about or exactly 300 mPa·s, more preferably the viscosity is within the range of about or exactly 300 mPa·s to about or exactly 1000 m Pa·s, Preferably, the fermented milk product is a yoghurt. The starter culture is herein referred to as a starter culture according to the invention. Preferably, the starter culture further comprises an excipient such as a cryoprotectant, a lyoprotectant, an antioxidant and/or a nutrient. In the starter culture according to according to the invention, the composition may be frozen, lyophilized, spray-dried, vacuum-dried, air dried, tray dried or may be in liquid form. The starter culture according to the invention may further comprise other lactic acid bacteria such as, but not limited to Lactococcus, Lactobacillus, Leuconostoc and Bifidobacterium, preferably Lactobacillus bulgaricus, Lactobacillus acidophilus, Lactobacillus casei, Lactobacillus rhamnosus, and Bifidobacterium, more preferably Lactobacillus delbrueckii subsp. bulgaricus.

Further provided is the use of the isolated Streptococcus thermophilus strain according to the invention, or the starter culture according to the invention for the production of a fermented milk product.

Further provided is a fermented milk product comprising a Streptococcus thermophilus strain according to the invention. Preferably, the fermented milk product has a pH of about or exactly 4.6 when freshly prepared and has a pH variation of less than 0.2 unit during storage of the freshly prepared milk product at 25° Celsius for 28 days, wherein the fermented milk product. The fermented milk product comprises an isolated Streptococcus thermophilus strain according to the invention or comprises a starter culture according to the invention. Preferably, the fermented milk product has a viscosity of at least about or exactly 300 mPa·s, preferably the viscosity is within the range of about or exactly 300 mPa·s to about or exactly 1000 mPa·s. In all embodiments herein, the fermented milk product may be any fermented milk product known to the person skilled in the art, such as, but not limited to regular yoghurt, low fat yoghurt, non fat yoghurt, kefir, dahi, ymer, buttermilk, butterfat, sour cream and sour whipped cream as well as fresh cheeses and quark, the fermented milk product preferably is a yoghurt. Such fermented milk product is herein referred to a as a fermented milk product according to the invention.

Further provided is a method for the production of a fermented milk product according to the invention, comprising fermenting a milk substrate with an isolated Streptococcus thermophilus strain according to the invention, or with a starter culture according to the invention. Said method to prepare a fermented milk product may be any method known to the person skilled in the art. Further provided is a fermented milk product according to the invention obtainable by said method.

### Definitions

As used herein the term “milk substrate” (also referred to as “milk base”) is the starting material, or starting substrate, for the fermentation process to provide a fermented milk product. It includes whole milk, skim milk, fat-free milk, low fat milk, full fat milk, lactose-free or lactose-reduced milk (produced by hydrolyzing the lactose by lactase enzyme to glucose and galactose, or by other methods such as nanofiltration, electro dialysis, ion exchange chromatography and centrifugation technology), concentrated milk or dry milk.

As used herein, “fat-free milk” is non-fat or skim milk product. Low-fat milk is typically defined as milk that contains from about 1% to about 2% fat. Full fat milk often contains 2% fat or more.

As used herein, the term “milk” encompasses milks from mammals and plant sources or mixtures thereof. Preferably, the milk is from a mammal source. Mammals sources of milk include, but are not limited to cow, sheep, goat, buffalo, camel, llama, mare and deer. In an embodiment, the milk is from a mammal selected from the group consisting of cow, sheep, goat, buffalo, camel, llama, mare and deer, and combinations thereof. Plant sources of milk include, but are not limited to, milk extracted from soy bean, pea, peanut, barley, rice, oat, quinoa, almond, cashew, coconut, hazelnut, hemp, sesame seed and sunflower seed. Soy bean milk is preferred. In addition, the term “milk” refers to not only whole milk, but also skim milk or any liquid component derived thereof.

As used herein, the term “fermented milk product” or “acidified dairy product” refers to products which are obtained by the multiplication of lactic acid bacteria in a milk base leading to a milk coagulum. The milk preparation used as raw material for the fermentation may be skimmed or non-skimmed milk, optionally concentrated or in the form of powder. Furthermore, this milk preparation may have been subjected to a thermal processing operation which is at least as efficient as pasteurization. The particular characteristics of the various fermented dairy products depend upon various factors, such as the composition of milk base, the incubation temperature, the lactic acid flora and/or non-lactic acid flora. Thus, fermented dairy products manufactured herein include, various types of regular yoghurt, low fat yoghurt, non fat yoghurt, kefir, dahi, ymer, buttermilk, butterfat, sour cream and sour whipped cream as well as fresh cheeses and quark.

The term “starter composition” or “starter culture” as used herein refers to a culture of one or more food-grade micro-organisms, in particular lactic acid bacteria, which are responsible for the acidification of the milk base. Starter cultures may be fresh (liquid), frozen or freeze-dried. Freeze dried cultures need to be regenerated before use. For the production of a fermented dairy product, the starter is usually added in an amount from 0.01 to 3%, preferably from 0.01 and 0.02% by weight of the total amount of milk base.

As used herein, the term “lactic acid bacteria” (LAB) or “lactic bacteria” refers to food-grade bacteria producing lactic acid as the major metabolic end-product of carbohydrate fermentation. These bacteria are related by their common metabolic and physiological characteristics and are usually Gram positive, low-GC, acid tolerant, non-sporulating, non-respiring, rod-shaped bacilli or cocci. During the fermentation stage, the consumption of lactose by these bacteria causes the formation of lactic acid, reducing the pH and leading to the formation of a protein coagulum. These bacteria are thus responsible for the acidification of milk and for the texture of the dairy product. As used herein, the term “lactic acid bacteria” or “lactic bacteria” encompasses, but is not limited to, bacteria belonging to the genus of Lactobacillus spp., Bifidobacterium spp., Streptococcus spp., Lactococcus spp., such as Lactobacillus delbruekii subsp. bulgaricus, Streptococcus thermophilus, Lactobacillus lactis, Bifidobacterium animalis, Lactococcus lactis, Lactobacillus casei, Lactobacillus plantarum, Lactobacillus helveticus, Lactobacillus acidophilus and Bifidobacterium breve.

The term “post-acidification” refers to the acidification profile of a bacterium or a bacterial culture according to the invention between pH 4.6 and 4.4. Post-acidification is the production of lactic acid occurring after the end of the fermentation. The end of fermentation is generally when the desired pH of 4.6 is reached. Viscosity measurements are herein performed using the measurements were done with the Anton Paar Rheometer, unless explicitly mentioned otherwise.

“Sequence identity” is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. “Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A “nucleic acid molecule” or “polynucleotide” (the terms are used interchangeably herein) is represented by a nucleotide sequence. A “polypeptide” is represented by an amino acid sequence. A “polypeptide” as used herein refers to any peptide, oligopeptide, polypeptide, gene product, expression product, or protein. A polypeptide is comprised of consecutive amino acids. The term “polypeptide” encompasses naturally occurring and synthetic molecules.

The sequence information as provided herein should not be so narrowly construed as to require inclusion of erroneously identified bases. The skilled person is capable of identifying such erroneously identified bases and knows how to correct for such errors.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a nucleic acid molecule or a peptide or polypeptide of a nucleic acid construct or vector or cell as defined herein may comprise additional component(s) than the ones specifically identified; said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 10% of the value.

All patent and literature references cited in the present specification are hereby incorporated by reference in their entirety.

Unless otherwise indicated each embodiment as described herein may be combined with another embodiment as described herein.

TABLE 1
Preferred mutations in view of wild-type strain Streptococcus thermophilus ASCC1275

| | | | | | SEQ ID | SEQ ID |
|---|---|---|---|---|---|---|
| | | | | | NO: | NO: |
| Mutation | Gene | | Nucleotide | Amino acid | wild type | wild-type |
| number | name | Gene ID | mutation | mutation | gene | protein |
| F-1 | hrtA_2 | F_01828 | c. 511G > A | p. Ala171Thr | 1 | 2 |
| F-2 | fni | F_01794 | c. 766C > T | p. Pro256Ser | 3 | 4 |
| F-3 | mapZ | F_00102 | c. 452C > T | p. Ser151Leu | 5 | 6 |
| F-4 | amiA_2 | F_00603 | c. 413C > T | p. Ala138Val | 7 | 8 |
| F-5 | ftsH | F_00365 | c. 1963G > A | p. Glu655Lys | 9 | 10 |
| F-6 | tmcAL | F_00805 | c. 355G > A | p. Ala119Thr | 11 | 12 |
| F-7 | artP_4 | F_01476 | c. 282G > A | p. Met94Ile | 13 | 14 |
| F-8 | dinB_3 | F_01475 | c. 758G > A | p. Arg253Lys | 15 | 16 |
| F-9 | groL | F_00133 | c. 62C > T | p. Thr21Ile | 17 | 18 |
| F-10 | aldC | F_01437 | c. 473G > A | p. Gly158Asp | 19 | 20 |
| F-11 | hsdR_4 | F_01638 | c. 1598C > T | p. Ala533Val | 21 | 22 |
| F-12 | glmU | F_01793 | c. 203C > T | p. Ala68Val | 23 | 24 |
| F-13 | ppaC | F_01981 | c. 283G > A | p. Val95Ile | 25 | 26 |
| F-14 | comFA | F_01994 | c. 1184G > A | p. Gly395Glu | 27 | 28 |
| F-15 | tyrS | F_00550 | c. 886G > A | p. Val296Ile | 29 | 30 |
| F-16 | trxB | F_00772 | c. 82G > A | p. Ala28Thr | 31 | 32 |
| F-17 | ctpE | F_00825 | c. 634G > A | p. Asp212Asn | 33 | 34 |
| F-18 | purL | F_00330 | c. 988G > A | p. Glu330Lys | 35 | 36 |
| F-19 | cysS | F_00263 | c. 758C > T | p. Ala253Val | 37 | 38 |
| F-20 | icd | F_01127 | c. 1003C > T | p. Leu335Phe | 39 | 40 |
| F-21 | pstB3_1 | F_01372 | c. 469G > A | p. Val157Ile | 41 | 42 |
| F-22 | rnhB | F_01460 | c. 605G > A | p. Arg202Gln | 43 | 44 |
| F-23 | deoC_2 | F_01534 | c. 154G > A | p. Gly52Arg | 45 | 46 |
| F-24 | tcyN_1 | F_00177 | c. 484G > A | p. Ala162Thr | 47 | 48 |
| F-25 | lytG | F_00232 | c. 292C > T | p. Pro98Ser | 49 | 50 |
| F-26 | ymcA | F_00314 | c. 290C > T | p. Ala97Val | 51 | 52 |
| F-27 | rpsF | F_00672 | c. 265C > T | p. His89Tyr | 53 | 54 |
| F-28 | carB | F_01840 | c. 1426G > A | p. Val476Ile | 55 | 56 |
| F-29 | dnaE2 | F_01209 | c. 613C > T | p. Pro205Ser | 57 | 58 |
| F-30 | malQ | F_01364 | c. 1055C > T | p. Thr352Ile | 59 | 60 |
| F-31 | glnB | F_01871 | c. 326G > A | p. Gly109Asp | 61 | 62 |
| F-32 | gdh | F_01931 | c. 218G > A | p. Gly73Asp | 63 | 64 |
| F-33 | kimA | F_00876 | c. 1214G > A | p. Gly405Asp | 65 | 66 |
| F-34 | mfd | F_00372 | c. 2618G > A | p. Gly873Asp | 67 | 68 |
| F-35 | dinB_1 | F_00766 | c. 770G > A | p. Gly257Glu | 69 | 70 |
| F-36 | dinB_1 | F_00766 | c. 853G > A | p. Glu285Lys | 71 | 72 |
| F-37 | pknD | F_00985 | c. 1343C > T | p. Ala448Val | 73 | 74 |
| F-38 | hisZ | F_01169 | c. 785C > T | p. Pro262Leu | 75 | 76 |
| F-39 | mdtG_1 | F_01724 | c. 182C > T | p. Ala61Val | 77 | 78 |
| F-40 | dnak | F_00229 | c. 1009C > T | p. Pro337Ser | 79 | 80 |
| F-41 | potA | F_00883 | c. 751G > A | p. Asp251Asn | 81 | 82 |
| F-42 | sthA | F_01345 | c. 203G > A | p. Gly68Asp | 83 | 84 |
| F-43 | alaS | F_01908 | c. 2579G > A | p. Ser860Asn | 85 | 86 |

TABLE 2
Preferred mutations in view of wild-type strain Streptococcus thermophilus
strain MN-ZLW-002

| | | | | | SEQ ID | SEQ ID |
|---|---|---|---|---|---|---|
| | | | | | NO: | NO: |
| Mutation | Gene | | Nucleotide | Amino acid | wild type | wild-type |
| number | name | Gene ID | mutation | mutation | gene | protein |
| C-1 | tmcAL | C_01613 | c. 766G > A | p. Ala256Thr | 87 | 88 |
| C-2 | hrtA | C_01388 | c. 313C > T | p. Gln105* | 89 | 90 |
| C-3 | fni | C_00613 | c. 391G > A | p. Glu131Lys | 91 | 92 |
| C-4 | artP_3 | C_01490 | c. 769G > A | p. Glu257Lys | 93 | 94 |
| C-5 | mapZ | C_00292 | c. 1396G > A | p. Ala466Thr | 95 | 96 |
| C-6 | ftsH | C_00027 | c. 1307C > T | p. Ser436Leu | 97 | 98 |
| C-7 | ftsH | C_00027 | c. 1331C > T | p. Ala444Val | 99 | 100 |
| C-8 | amiA_2 | C_01446 | c. 1573G > A | p. Ala525Thr | 101 | 102 |
| C-9 | leuC | C_01205 | c. 1144T > A | p. Leu382Met | 103 | 104 |
| C-10 | pyrDA | C_01210 | c. 631C > T | p. Pro211Ser | 105 | 106 |
| C_11 | btuD_4 | C_01964 | c. 1120C > T | p. Arg374Cys | 107 | 108 |
| C-12 | polC | C_00107 | c. 1232C > T | p. Pro411Leu | 109 | 110 |
| C-13 | IMPDH_1 | C_00414 | c. 214G > A | p. Gly72Arg | 111 | 112 |
| C-14 | fabM | C_00439 | c. 518C > T | p. Ala173Val | 113 | 114 |
| C-15 | atpB | C_00533 | c. 14G > A | p. Ser5Asn | 115 | 116 |
| C-16 | yabA | C_00546 | c. 97G > A | p. Gly33Arg | 117 | 118 |
| C-17 | thrS | C_00625 | c. 319G > A | p. Asp107Asn | 119 | 120 |
| C-18 | nylA | C_01009 | c. 301G > A | p. Asp101Asn | 121 | 122 |
| C-19 | pyrK | C_01023 | c. 698G > A | p. Gly233Glu | 123 | 124 |
| C-20 | oppD_4 | C_01443 | c. 212G > A | p. Gly71Glu | 125 | 126 |
| C-21 | adeQ | C_00386 | c. 503G > A | p. Gly168Asp | 127 | 128 |
| C-22 | argD | C_00522 | c. 536G > A | p. Gly179Asp | 129 | 130 |
| C-23 | leuB | C_01207 | c. 374G > A | p. Gly125Asp | 131 | 132 |
| C-24 | rluB_3 | C_01501 | c. 607G > A | p. Ala203Thr | 133 | 134 |
| C-25 | purF | C_00064 | c. 1151C > T | p. Ala384Val | 135 | 136 |
| C-26 | radA | C_01821 | c. 335C > T | p. Thr112Ile | 137 | 138 |
| C-27 | urel | C_00334 | c. 262C > T | p. Arg88Cys | 139 | 140 |
| C-28 | btuD_1 | C_00596 | c. 125G > A | p. Gly42Asp | 141 | 142 |
| C-29 | scmP | C_00605 | c. 278G > A | p. Cys93Tyr | 143 | 144 |
| C-30 | glmM | C_01269 | c. 553G > A | p. Ala185Thr | 145 | 146 |
| C-31 | gyrA | C_01296 | c. 2128G > A | p. Val710Ile | 147 | 148 |
| C-32 | trpF | C_01586 | c. 248G > A | p. Gly83Glu | 149 | 150 |
| C-33 | mprF | C_01274 | c. 275C > A | p. Thr92Lys | 151 | 152 |
| C-34 | mutS | C_00080 | c. 2407C > T | p. Pro803Ser | 153 | 154 |
| C-35 | bgIH_4 | C_01627 | c. 742G > A | p. Asp248Asn | 155 | 156 |

TABLE 3
Preferred nucleotide sequences for genes in the Streptococcus
thermophilus strains of the invention and preferred amino
acid sequences for in the Streptococcus thermophilus strains
of the invention (hrtA sequence seq id no 170 is truncated).

| | | SEQ ID NO: | SEQ ID NO: |
|---|---|---|---|
| | | of the gene | of the protein |
| Mutated | Mutated | (DNA/nucleotide | (protein/amino |
| gene | protein | sequence) | acid sequence) |
| hrtA_2 | HRTA_2 | 157 | 158 |
| aldC | ALDC | 159 | 160 |
| purL | PURL | 161 | 162 |
| rnhB | RNHB | 163 | 164 |
| dnaE2 | DNAE2 | 165 | 166 |
| sthA | STHA | 167 | 168 |
| hrtA | HRTA | 169 | 170 |
| pyrk | PYRK | 171 | 172 |
| oppD_4 | OPPD_4 | 173 | 174 |
| argD | ARGD | 175 | 176 |
| btuD_1 | BTUD_1 | 177 | 178 |
| scmP | SCMP | 179 | 180 |
| mprF | MPRF | 181 | 182 |

The following examples are offered for illustrative purposes only, and are not intended to limit the scope of the present invention in any way.

### Example 1

Isolation of S. thermophilus Strains CBS149207 and CBS149208.

Using classical strain improvement (NTG mutagenesis), strains CBS149207 and CBS149208 were isolated. Strain CBS149207 was obtained by mutagenesis of a commercial strain obtainable from DSM Food Specialties in the Netherlands and compared with strain Streptococcus thermophilus ASCC1275. Strain CBS149208 was obtained by mutagenesis of a commercial strain obtainable from DSM Food Specialties in the Netherlands and compared with Streptococcus thermophilus MN-ZLW-002. Selection of mutant strains was done using a step-wise approach. The recovered single mutated bacterial strains were assessed upon acidification, post acidification (PA) and textural properties. Upon each screening step, the candidates with the right performance were selected. Mutant strains with equal acidification speed, lower PA, and equal texturing properties to that of the parent strain were selected. Selected mutant strains were further validated in down-scaled milk fermentation either as single or co-cultivations.

Strain CBS149207 was analysed genetically and phenotypically and was deposited at the Westerdijk Fungal Biodiversity Institute, Uppsalalaan 8, 3548 CT, the Netherlands. Strain CBS149208 was analysed genetically and phenotypically and was deposited at the Westerdijk Fungal Biodiversity Institute, Uppsalalaan 8, 3548 CT, the Netherlands. Deposits were made under the regulations of the Budapest Treaty.

### Example 2

Production of Yoghurt Using Strains CBS149207 and CBS149208 (Laboratory Scale)

Strains CBS149207 and CBS149208 were used for the production of yoghurt using routine methods known by the person skilled in the art and acidification, post-acidification and viscosity were determined. The milk used for this example was UHT treated full-fat milk (3.4% Protein, 3.6% Fat) was used for this experiment with extra addition of 7% sucrose and 10 ppm Sodium Formate. Acidification to pH 4.6 occurred for both strains within 5 hours. During the first 7 days shelf life at 25° C., the pH lowered to between pH 4.2 and pH 4.3 and remained between pH 4.2 and pH 4.3 up to and including day 14 of shelf life at 25° C. In contrast, the wild-type strains demonstrated considerable post-acidification during shelf life at 25° C., down to below pH 4 already during the first 7 days of shelf life at 25° C. The viscosity of the product obtained by either the wild-type or the newly isolated CBS149207 and CBS149208 strains was between 10000 and 25000 mPa·s using the Brookfield method.

### Example 3

Production of Yoghurt Using Strains CBS149207 and CBS149208 (Pilot Scale)

Strains CBS149207 and CBS149208 were used for the production of yoghurt using routine methods known by the person skilled in the art and acidification, post-acidification and viscosity were determined. The milk use for this example was yoghurt milk (5 min 92° C. mild pasteurization) with 3.0% fat, 2.7% protein, and 7% sucrose. Acidification to pH 4.5 occurred for both strains within 5 hours. During the first 7 days shelf life at 20° C., the pH lowered to between pH 4.35 and pH 4.4 and remained between pH 4.35 and pH 4.4 up to and including day 28 of shelf life at 20° C. In contrast, the wild-type strains demonstrated considerable post-acidification during shelf life at 20° C., down to below pH 4.25 already during the first 7 days of shelf life at 20° C. The viscosity of the product obtained by either the wild-type or the newly isolated CBS149207 and CBS149208 strains was between 300 and 1000 mPa·s. These measurements were done with the Anton Paar Rheometer.

### Example 4

Measurement of Acetoin and Acetolactate Levels in Milk Fermentations

In this experiment, the changes in acetoin and acetolactate production of mutant CBS149207 vis a vis its parent were determined. 12% (w/v) RSM (reconstituted skim milk) was pasteurized by heating in a water bath for 15 minutes at 90° C., followed by 30 minutes at 85° C. After pasteurization, the milk was quickly cooled in ice-water and kept at 4° C. The pasteurized milk was inoculated with either the parent strain (parent) or the CBS149207 (mutant), and incubated at 42° C. The pH was continuously monitored using a CINAC apparatus (Ysebaert, France). From duplicate milk acidifications, samples were taken once the parent reached pH 5.00 (pH1), pH 4.75 (pH2), pH 4.50 (pH3) and pH 4.25 (pH4) for milk fermented with either parent or with the mutant. The acidification curves showed a levelling off of the pH for mutant compared with parent especially below pH 4.5. This phenotype is correlated to the mild behavior of strain CBS149207, as it produces less lactic acid than the wildype.

As a reference, a blanco, non-inoculated pasteurized 12% RSM milk sample was prepared for subsequent measurement.

Subsequently the samples were measured with NMR.

Strikingly, the acetoin levels in the milk were much lower (ca. 50% lower) for the mutant compared to the parent (see table 4 below). This was an indication that the mutation in aldC gen in the mutant resulted in a loss of activity of the gene product, i.e. the α-Acetolactate Decarboxylase AldC) protein, an enzyme having α-Acetolactate Decarboxylase activity.

The reaction for AldC reads as: α-acetolactate a acetoin+CO₂.

This was further substantiated by the more pronounced accumulation of α-acetolactate in the milk of the mutant which is the substrate of AldC. These results indicate that the coding enzyme for the mutated aldC gene in the mutant displayed loss of activity. Consumed lactose levels seemed similar between parent and mutant. Lactic acid levels were generally lower at the last sampling time point (pH4) for the mutant but that fits with the earlier levelling off of the acidification compared to the parent.

TABLE 4
extracellular metabolite levels pasteurized milk fermentation
(n.m., not measured; b.d., below detection limit)

| Sample | parent (1) | parent (2) | CBS149207 (1) | CBS149207 (2) | Milk |
|---|---|---|---|---|---|
| | Lactose (g/L) | | | | |
| pH1 | 53.2 | 53.5 | 53.6 | 52.9 | 66.8 |
| pH2 | 51.9 | 51.9 | 51.3 | 51.1 | |
| pH3 | 49.1 | 48.9 | n.m. | n.m. | |
| pH4 | 44.1 | 44.9 | 46.2 | 45.6 | |
| | Lactic acid (g/L) | | | | |
| pH1 | 4.6 | 4.3 | 4.5 | 4.5 | b.d. |
| pH2 | 6.0 | 5.7 | 5.8 | 5.8 | |
| pH3 | 7.2 | 7.0 | n.m. | n.m. | |
| pH4 | 9.4 | 9.3 | 8.2 | 8.5 | |
| | Acetoin (mg/L) | | | | |
| pH1 | 51.9 | 50.5 | 21.0 | 19.8 | b.d. |
| pH2 | 62.8 | 61.3 | 30.0 | 32.6 | |
| pH3 | 66.0 | 65.0 | n.m. | n.m. | |
| pH4 | 73.1 | 70.6 | 46.1 | 43.1 | |
| | α-acetolactate (mg/L) | | | | |
| pH1 | b.d. | b.d. | 22.0 | 21.2 | b.d. |
| pH2 | b.d. | 5.3 | 14.0 | 13.2 | |
| pH3 | b.d. | b.d. | n.m. | n.m. | |
| pH4 | b.d. | b.d. | b.d. | b.d. | |

### Example 5

Acidification Single Streptococcus Strains

12% (w/v) RSM (reconstituted skim milk) was pasteurized by heating in a water bath for 15 minutes at 90° C., followed by 30 minutes at 85° C. After pasteurization, the milk was quickly cooled in ice-water and kept at 4° C. The pasteurized milk was inoculated with 2% inoculum and incubated at 42° C. for 24 hours. The inoculum used was an overnight culture (16-18 hrs at 42° C.) in 12% (w/v) RSM of each tested strain:
parent of CBS149207 or CBS149207
parent of CBS149208 or CBS149208. The pH was continuously monitored using a CINAC apparatus (Ysebaert, France).

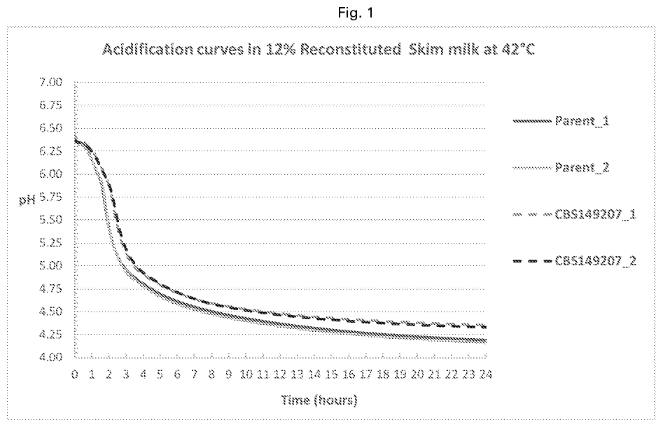

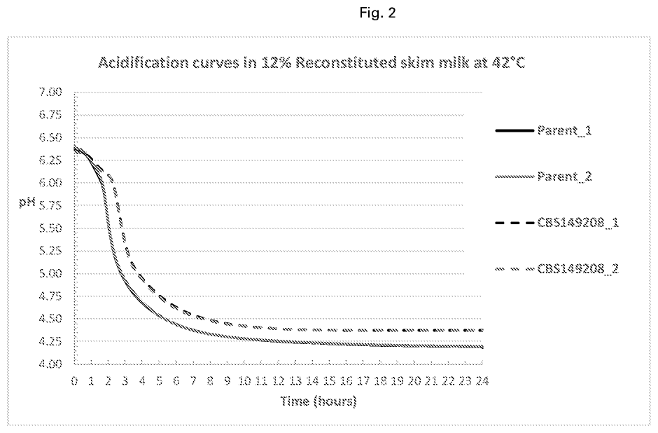

The results are illustrated in Table 5 and FIGS. 1 and 2. As illustrated the mutant strains can still acidify well in milk. The acidification potential of the mutants is slightly lower than that of the parent strains, but they are still very good acidifiers.

TABLE 5

| | TTR pH 4.60 (min) | | | | pH 24 h | | | |
|---|---|---|---|---|---|---|---|---|
| | run | run | | % | run | run | | % |
| STRAIN | 1 | 2 | Average | CV | 1 | 2 | Average | CV |
| Parent | 373 | 346 | 360 | 0.053 | 4.2 | 4.2 | 4.18 | 0.005 |
| CBS149207 | 466 | 462 | 464 | 0.006 | 4.4 | 4.3 | 4.34 | 0.004 |
| Parent | 271 | 272 | 272 | 0.003 | 4.2 | 4.2 | 4.19 | 0.002 |
| CBS149208 | 378 | 369 | 374 | 0.017 | 4.4 | 4.4 | 4.37 | 0.001 |

### Example 6

Texture Single Strains and in Co-Culture with Lactobacillus delbrueckii C37

12% (w/v) RSM (reconstituted skim milk) was pasteurized by heating in a water bath for 15 minutes at 90° C., followed by 30 minutes at 85° C. After pasteurization, the milk was quickly cooled in ice-water and kept at 4° C.

UHT milk was bought from the supermarket (full fat millk). 7% Sucrose and 10 ppm Sodium formate were added to the milk.

The milk was inoculated with 2% ST inoculum and 0.2% Lb and incubated at 42° C. for 24 hours. The inoculum used was an overnight culture (16-18 hrs at 42° C.) in 12% (w/v) RSM of each tested strain: parent or CBS149207 or CBS149208 or Lactobacillus delbrueckii C37. The pH was continuously monitored using a CINAC apparatus (Ysebaert, France). When reaching pH 4.6 samples were quickly put on ice-water for 30 minutes and kept at 4° C. After 16-18 samples were analysed using the Brookfield method.

The results are illustrated below in table 6. As illustrated the mutant strains are very good texturizers. Texturizing potential is as good as the parent strain.

TABLE 6

| | | Av viscosity | |
|---|---|---|---|
| | Strain | (mpa/s) | Stdev |
| 12% Reconstituted | Parent | 19750 | 330 |
| skim milk | CBS149207 | 21095 | 464 |
| | CBS149208 | 28718 | 705 |
| UHT milk + 7% | Parent + Lb | 13777 | 151 |
| sucrose + 10 | CBS149207 + Lb | 13565 | 323 |
| ppm NaFormate | CBS149208 + Lb | 18853 | 14 |

### Example 7

Viability in Acidified Milk or Yoghurt Sample of Single Streptococcus Strains

To obtain samples for viability studies 12% (w/v) RSM (reconstituted skim milk) was pasteurized by heating in a water bath for 15 minutes at 90° C., followed by 30 minutes at 85° C. After pasteurization, the milk was quickly cooled in ice-water and kept at 4° C. The pasteurized milk was inoculated with 2% inoculum and incubated at 42° C. for 24 hours. Samples for viability studies were taken directly after inoculation (INOC). The inoculum used was an overnight culture (16-18 hrs at 42° C.) in 12% (w/v) RSM of each tested strain: parent CBS149207 or CBS149207, parent CBS149208 or CBS149208. The pH was continuously monitored using a CINAC apparatus (Ysebaert, France). In some cases, after pH 4.6 was reached samples were processed towards yoghurt as known by persons in the art and cooled to 4° C. Samples for viability studies were taken once pH 4.6 was reached (EoF). In some cases, the acidification of milk at 42° C. was allowed to continue until 24 h after which samples were taken for viability studies (24H samples). The processed yoghurt samples were stored at 4° C. for 1 to 5 days. Samples for viability studies were taken of these yoghurt samples, as well, Day1 and Day5 respectively. In summary for all four strains the following samples were available: INOC, EoF, 24H, Day1 and Day5, as explained above. The samples were processed accordingly, and live cells were measured with flow cytometry according to ISO 19344-2015 protocol and results were expressed as cells/mL. The results are found in Table 7 below.

The results indicated that for both CBS149207 and CBS149208 a comparable amount of viable cells was found as with their respective parent strains indicating that the preferential low post-acidification phenotype in milk and yoghurt observed for CBS149207 and CBS149208 was not the result of a complete loss of viable cellsunder the relevant conditions. The viability of the new mutant strains CBS149207 and CBS149208 was still good. The CBS149208 showed a drop of viable cells as measured in the 24 hour samples, but the parent of CBS149208 showed a similar dynamic.

TABLE 7

| sample # | timepoint | strain | Live cells (cells/ml) |
|---|---|---|---|
| 1 | Inoc | parent CBS149207 | 3.7E+07 |
| 2 | EoF | parent CBS149207 | 1.6E+09 |
| 3 | 24 h | parent CBS149207 | 7.5E+08 |
| 4 | Day 1 | parent CBS149207 | 1.4E+09 |
| 5 | Day 5 | parent CBS149207 | 1.4E+09 |
| 6 | Inoc | CBS149207 | 2.2E+07 |
| 7 | EoF | CBS149207 | 1.5E+09 |
| 8 | 24 h | CBS149207 | 1.1E+09 |
| 9 | Day 1 | CBS149207 | 1.8E+09 |
| 10 | Day 5 | CBS149207 | 1.6E+09 |
| 11 | Inoc | parent CBS149208 | 1.4E+07 |
| 12 | EoF | parent CBS149208 | 1.3E+09 |
| 13 | 24 h | parent CBS149208 | 7.6E+07 |
| 14 | Day 1 | parent CBS149208 | 1.3E+09 |
| 15 | Day 5 | parent CBS149208 | 1.2E+09 |
| 16 | Inoc | CBS149208 | 2.3E+07 |
| 17 | EoF | CBS149208 | 1.2E+09 |
| 18 | 24 h | CBS149208 | 1.4E+07 |
| 19 | Day 1 | CBS149208 | 1.0E+09 |
| 20 | Day 5 | CBS149208 | 8.0E+08 |

1. An isolated Streptococcus thermophilus strain, wherein the activity of a native ABC transporter complex ATP binding protein or a component thereof, is reduced, impaired, or disabled.

2. The isolated Streptococcus thermophilus strain according to claim 1, wherein the ABC transporter complex ATP binding protein is an ATPase.

3. The isolated Streptococcus thermophilus strain according to claim 1, wherein the ABC transporter complex ATP binding protein is a HrtA protein and/or a HrtA_2 protein and/or another hemin import ATP-binding protein.

4. An isolated Streptococcus thermophilus strain CBS149207 and mutants and variants thereof or an isolated Streptococcus thermophilus strain CBS149208 and mutants and variants thereof, wherein said mutants and variants thereof optionally comprise at least one mutation selected from the groups of mutations consisting of:
F1 to F43, optionally selected from the group consisting of F-1, F-10, F-18, F-22, F-29, and F-42 and combinations thereof, each as depicted in Table 1; and
C1 to C35, optionally selected from the group consisting of C-2, C-19, C-20, C-22, C-28, C-29 and C-33 and combinations thereof, each as depicted in Table 2.

5. An isolated Streptococcus thermophilus strain according to claim 1, wherein the mutants and variants demonstrate the same or substantially the same milk acidification characteristics as Streptococcus thermophilus strain CBS149207 or CBS149208 in the preparation of a fermented milk product.

6. An isolated Streptococcus thermophilus strain according to claim 1, wherein the milk acidification characteristics in the preparation of a fermented milk product comprise acidification of a milk substrate to a pH of about or exactly 4.6 in less than 5 hours and a pH variation of less than 0.2 unit during storage of the freshly prepared fermented milk product at 25° Celsius for 28 days.

7. An isolated Streptococcus thermophilus strain according to claim 1, wherein the milk acidification characteristics further comprise viscosity of the fermented milk product of at least about 300 mPa·s, optionally the viscosity is within the range of about or exactly 300 mPa·s to about or exactly 1000 mPa·s and optionally the viscosity is within the range of about or exactly 400 mPa·s to about or exactly 550 m Pa·s.

8. An isolated Streptococcus thermophilus strain having a genome with at least 80% sequence identity to the genome of Streptococcus thermophilus ASCC1275 or Streptococcus thermophilus MN-ZLW-002, wherein the isolated Streptococcus thermophilus strain demonstrates the same or substantially the same milk acidification characteristics as Streptococcus thermophilus strain CBS149207 or CBS149208 in the preparation of a fermented milk product, wherein the milk acidification characteristics in the preparation of a fermented milk product comprise acidification of a milk substrate to a pH of about or exactly 4.6 in less than 5 hours and a pH variation of less than 0.2 unit during storage of the freshly prepared milk product at 25° Celsius for 28 days, and wherein the Streptococcus thermophilus strain comprises at least one mutation selected from the groups of mutations consisting of:
F1 to F43, optionally selected from the group consisting of F-1, F-10, F-18, F-22, F-29, and F-42 and combinations thereof, each as depicted in Table 1; and
C1 to C35, optionally selected from the group consisting of C-2, C-19, C-20, C-22, C-28, C-29 and C-33 and combinations thereof, each as depicted in Table 2.

9. An isolated Streptococcus thermophilus strain according to claim 8, wherein the milk acidification characteristics further comprise viscosity of the fermented milk product of at least about 300 mPa·s, optionally the viscosity is within the range of about or exactly 300 mPa·s to about or exactly 1000 mPa·s.

10. An isolated Streptococcus thermophilus strain according to claim 1, wherein the strain is a mutant or variant that is obtained by mutagenesis or by genome editing and subsequent selection for the milk acidification characteristics.

11. A method for the production of an isolated Streptococcus thermophilus strain comprising:
a. providing a parent Streptococcus thermophilus strain,
b. mutagenizing the Streptococcus thermophilus strain,
c. selecting for a mutant with improved milk acidification characteristics, optionally at least one of the milk acidification characteristics as defined in claim 6, and
d. isolating the mutant with the improved milk acidification characteristics.

12. A method according to claim 11, wherein the parent Streptococcus thermophilus strain is ASCC1275 or is MN-ZLW-002, or wherein the parent strain is a Streptococcus thermophilus strain with substantially the same milk acidification characteristics as ASCC1275 or is MN-ZLW-002.

13. An isolated Streptococcus thermophilus strain obtainable by the method according to claim 11.

14. A starter culture for the production of a fermented milk product optionally having elongated shelf life and/or optionally having the ability to be stored at elevated temperature, comprising an isolated Streptococcus thermophilus strain according to claim 1.

15. A starter culture according to claim 14, wherein the fermented milk product has a pH of about or exactly 4.6 when freshly prepared and has a pH variation of less than 0.2 unit during storage of the freshly prepared milk product at 25° Celsius for 28 days.

16. A starter culture according to claim 14, wherein the fermented milk product has a viscosity of at least about or exactly 300 mPa·s, optionally the viscosity is within the range of about or exactly 300 mPa·s to about or exactly 1000 mPa·s, and/or wherein the fermented milk product optionally is a yoghurt.

17. A starter culture according to claim 14, further comprising an excipient such as a cryoprotectant, a lyoprotectant, an antioxidant and/or a nutrient.

18. A starter culture according to claim 14, wherein the starter culture is a composition or is a kit of parts.

19. A starter culture according to claim 14, wherein the composition is frozen, lyophilized, spray-dried, vacuum-dried, air dried, tray dried or is in liquid form.

20. A starter culture according to claim 14, further comprising a Lactobacillus strain, optionally a Lactobacillus bulgaricus, a Lactobacillus acidophilus, a Lactobacillus casei, a Lactobacillus rhamnosus, and/or a Bifidobacterium, optionally a Lactobacillus delbrueckii subsp. bulgaricus.

21. The use of the isolated Streptococcus thermophilus strain according to claim 1, or the starter culture for the production of a fermented milk product.

22. A fermented milk product optionally having a pH of about or exactly 4.6 when freshly prepared and having a pH variation of less than 0.2 unit during storage of the freshly prepared milk product at 25° Celsius for 28 days, wherein the fermented milk product comprises an isolated Streptococcus thermophilus strain according to claim 1, or comprises the starter culture.

23. A fermented milk product according to claim 22, wherein the fermented milk product has a viscosity of at least about or exactly 300 mPa·s, optionally the viscosity is within the range of about or exactly 300 mPa·s to about or exactly 1000 mPa·s, and wherein the fermented milk product optionally is a yoghurt.

24. A method for the production of a fermented milk product according to claim 22, comprising fermenting a milk substrate with an isolated Streptococcus thermophilus strain, or with the starter culture.

25. A fermented milk product obtainable by a method according to claim 24.


SEQUENCE LISTING (182 sequences)

SEQ ID NO: 1 - DNA, 675 bp, synthetic construct
  source 1..675: mol_type = other DNA
atgtcaaatctcattttcgactcagtcacaaaaatttttggtgaggggggtagtaagtacgtagcactagaaaatattaatttcgaagcagaaagcggtcagcttattttagttgtagggccatcaggatcaggaaaaactacatttttgactatagcaggcggactacagacaccaactaatggtgacgtaaaaataaatgacagtacaattaacagtttgtccaaaaaacaacaaacaaaattaagattggaaaaaataggatttattttacaatcttataacttagtcccatttttaacggtcgaagaacagtttaaattcgtagataaactaaaaaaacaaaatttgactgaacaaaaattgcatgagcttctttctgatttaggacttttagaactgctaaaaaaatatcctaatcaactttctggtggacaaaagcagcgtgtggcaattgctagagcattatacactgatcctgactatattttagcagatgaacccacagccgcattagatacagatagatccatgaaagtaatcgagttactcagagatttagcgcacaaaagaaataaaattattattgtagttactcatgacttaaggttaaaagatatggcagacaaagtttatcagataattgacggtaaaatgacaatcctggagaagtaa

SEQ ID NO: 2 - protein, 224 aa, synthetic construct
MSNLIFDSVTKIFGEGGSKYVALENINFEAESGQLILVVGPSGSGKTTFLTIAGGLQTPTNGDVKINDSTINSLSKKQQTKLRLEKIGFILQSYNLVPFLTVEEQFKFVDKLKKQNLTEQKLHELLSDLGLLELLKKYPNQLSGGQKQRVAIARALYTDPDYILADEPTAALDTDRSMKVIELLRDLAHKRNKIIIVVTHDLRLKDMADKVYQIIDGKMTILEK

SEQ ID NO: 3 - DNA, 1008 bp, synthetic construct
  source 1..1008: mol_type = other DNA
atgacaaatcgtaaagacgatcacatccgttatgccctcaagtaccagtcgccctacaatagttttgacgatatggaacttatccacaagtctctaccgacttatgatttagatcagattgacttgtccacacattttgctggacgagattggaaattccccttttatatcaatgccatgacaggtggatctgccaaaggtggggctgttaatcgaaaactagctgaggtggctagccgaacaggtattctcatggtgacgggttcctacagtgctgctctaaaaggagaggcaccagaatcctttgactatcgtaatgagtttccagacttagaccttgcgaccaatatcggtgttgataagtctgtggacctggggattaagacggtggaagctatggatccagtctttttacagctccatgttaatcttatgcaggagcttctcatgcctgagggagagcgtattttccacacttggaaggaaaatgtggcggcttatgcacagaaaatagaggttcctcttgtgcttaaggaggttggctttggtatggatgagaaaaccattcgctacgccatgtctcagggcatcaagactgtggacatctcaggtcgtggtggaactagctttgcctatattgaaaatagtcgaggtggtaactgtgattacctaaatgactggggacaaagtacggttcagacccttttgcaagcccaagaccttcgtgaggaggtggaaatcctagcctctggtggcgtgcgcaatccacttgatatggttaaatgtctggtcttaggagcaaaaggtgtaggcctctcacgaacagttcttgaattggtggaacgttatcctgtggataaggttgtggctatcgtcaatggctggaaggatgaccttcgccttatcatgtgtgcccttgattgtcgcactattgatgagctcaaatctgtagattacatcttatatggaaagttacaagggacttatgtcaaacaagagaagtcataa

SEQ ID NO: 4 - protein, 335 aa, synthetic construct
MTNRKDDHIRYALKYQSPYNSFDDMELIHKSLPTYDLDQIDLSTHFAGRDWKFPFYINAMTGGSAKGGAVNRKLAEVASRTGILMVTGSYSAALKGEAPESFDYRNEFPDLDLATNIGVDKSVDLGIKTVEAMDPVFLQLHVNLMQELLMPEGERIFHTWKENVAAYAQKIEVPLVLKEVGFGMDEKTIRYAMSQGIKTVDISGRGGTSFAYIENSRGGNCDYLNDWGQSTVQTLLQAQDLREEVEILASGGVRNPLDMVKCLVLGAKGVGLSRTVLELVERYPVDKVVAIVNGWKDDLRLIMCALDCRTIDELKSVDYILYGKLQGTYVKQEKS

SEQ ID NO: 5 - DNA, 1863 bp, synthetic construct
  source 1..1863: mol_type = other DNA
gtgtcagaagataaaacacaaaatggctatgaagaaagccaagagttagatttccaagatgccaaggaaatgacagtcggtgaggctgtccgcaaagaagctgagattaatgctggtgtaaccgaaacagatagtattttggataagtatattaagcaacatcgtgaagaagttacctcacaaaaattttcaaagaaaattgaagcagatggcgatacatcacccttggatgccttcattcaaaagcaacgtcaggaatttgctgattcaggtttgattggtcaaacattggctaatgagtcaactaactcaacgactgctgacgaaacagtaacacctattacatctggttttggcacaactgaaactaaggcagatgatactcaagctcctgttgactctgaaatcactgtgaaaccagaaagtgaaagctctgaaaccatcataacatcaactaatgctgatcgttttatcacgtcagaagcagaaaaatttgatcttggtgaagcccttactgcgacgactacctcaactaatcaacaagttgataacactgctatggtagacaatgttgatgatccggaaccagaaagtactttacctgctgatgacaagtcttctgaaccacaagcctctaaacctctgttggaagatgttggagtctcagaaacaatcgactcagacgctattgccacaacagttgctggtgtaacaggagttaaagctgatgaagcaaaagcgacaccaaaagtgtctatgacagttagtcgtccatcagcagaagataaaatctcttcagggtatatttctccatctcataaaggtgtctttgatggtgatattccagtttatcgccgtaagggtgttgttatcggtgctctcacggttcttgctctagctataattggtgggagctatgctctttataaagtaacacatagccaatctgcgaaaactacaagcactgcttcttcagcagtgatatcatcatcaagcaagggcgcatcagcttctgataataaagccttcgaagacatgtataagaatttcttcacagatgatgagcaaacaaaactagctaatgatcaatttggtaagctatcagacttggaaaaactacttaaaaaattggaaaaaaccaagtattatgatgcagctaagaccaaatacgataacttgaagaaacaaatcgaagctattgaaaaagtcaatagtaagtatgaatcagatgctcttgtagacggtagctataatgcctctatctcagttaaatctgatgcaaactttaatgatctttcagaacgtgtaacaaatactggcaatgctagtcttgatagcattatccaagaagtcattaagggcggtaaaacacagcttgaagaaaagggtaaggctgcaagtgcaacgtctgcagtgacagcttctgaaagtgttaataccgctactccttcgggagacaacacatctggagcagctaattcaggtgtaacaggtgcggctggtggagtttctagtggtacagctgcaacttcaacagttgtgacacgtggcatcatgaattacaatccatctatccttcaacgtgatcgctctcgtgtgccttataatgcaaacgtagtagcggatacaagtaatccagcttggacttgggctgatggtgttcttgataagatcattgcaacatctcactcacgtgggtacttctcaggagataactttatccttgagccagtcaacatcatcaacggaaatggttattacaacatgtaccttccagatggaacttatctctttagtatcaactgtaagacaggttactatgtaggcaatggttcaggtcactcagataaacttgattattaa

SEQ ID NO: 6 - protein, 620 aa, synthetic construct
VSEDKTQNGYEESQELDFQDAKEMTVGEAVRKEAEINAGVTETDSILDKYIKQHREEVTSQKFSKKIEADGDTSPLDAFIQKQRQEFADSGLIGQTLANESTNSTTADETVTPITSGFGTTETKADDTQAPVDSEITVKPESESSETIITSTNADRFITSEAEKFDLGEALTATTTSTNQQVDNTAMVDNVDDPEPESTLPADDKSSEPQASKPLLEDVGVSETIDSDAIATTVAGVTGVKADEAKATPKVSMTVSRPSAEDKISSGYISPSHKGVFDGDIPVYRRKGVVIGALTVLALAIIGGSYALYKVTHSQSAKTTSTASSAVISSSSKGASASDNKAFEDMYKNFFTDDEQTKLANDQFGKLSDLEKLLKKLEKTKYYDAAKTKYDNLKKQIEAIEKVNSKYESDALVDGSYNASISVKSDANFNDLSERVTNTGNASLDSIIQEVIKGGKTQLEEKGKAASATSAVTASESVNTATPSGDNTSGAANSGVTGAAGGVSSGTAATSTVVTRGIMNYNPSILQRDRSRVPYNANVVADTSNPAWTWADGVLDKIIATSHSRGYFSGDNFILEPVNIINGNGYYNMYLPDGTYLFSINCKTGYYVGNGSGHSDKLDY

SEQ ID NO: 7 - DNA, 474 bp, synthetic construct
  source 1..474: mol_type = other DNA
gtgaatcaagtgcaatctctcaaacagtctattgaagccacacttggaaaaaagaatgttgtcattgatatccatcaattgtcagccaacgatttctacaatattacttactatgcttcgaatgcagcagcagagtattgggacttgtcagttggtgttgcttgggagcctaattacctcgatccatccacatatcttgatgttttgaaaacgactagtagcgagaacaccaagtcctttatgggttacgacaatccaaatagtcaagctgtggaaaaagtaggtcttaaagaatatgaccaattagttgaggatgcttctaaggaaaccacagatttaaaagttcgttatgaaaaatatgctaaagctcaagcatggttgaaagatagtgccctctatcttccagctaccgcttatagtggtgcagcaacggtagtttctcgcatacagccattctcaggagcttacgcttaa

SEQ ID NO: 8 - protein, 157 aa, synthetic construct
VNQVQSLKQSIEATLGKKNVVIDIHQLSANDFYNITYYASNAAAEYWDLSVGVAWEPNYLDPSTYLDVLKTTSSENTKSFMGYDNPNSQAVEKVGLKEYDQLVEDASKETTDLKVRYEKYAKAQAWLKDSALYLPATAYSGAATVVSRIQPFSGAYA

SEQ ID NO: 9 - DNA, 1968 bp, synthetic construct
  source 1..1968: mol_type = other DNA
atgaataataaaaataataatagcttcttacggaatgctttcctttacatcgtaatgattattgcggttgtaacggcagtacagtattttgttggaggaagtcaaagtccaagtcaacaattatcttatacccagttgattaagaaaattgaagatggtaaggtaaaatcaattacctatcaacctaatggtagtatcattgaagttaaaggagactacaaaaaggctgaaaaggttgataatgacctaagtttgccatttttaggtggggcatcgtcagagacacaaagatttacatcaactgtgcttcataatgaaacgataatacaggcattacagtcagcttcagaaaaagctaatgttaatatcaaaatgatacgtgaaagttcaagtggtacttggatttcgtatctctttagttaccttcctttgattggtattgctatttttttcatttttatgatgaatcaaggtggtaatcgcggtgccatgaactttggccgtaatcgtgctaaagctcagtcaaaagaaaatattaaagttcgctttacagatgtagccggtgcagaagaagaaaaagaagaacttgtagaaatagtagatttcctcaaaaatccacgtaaatataaagctttgggtgcacgtattcctaaaggagtcctcctcgaaggccctccaggaacaggtaaaaccctccttgctaaagctgtggctggtgaagctgatgtaccattctttagcatctcaggttctgattttgttgaaatgtttgtcggtgtcggagctagccgtgtacgtagtctgtttgaagatgctaaaaaggcagagcgtgctattatctttattgatgaaattgatgctgttggtcgtcgtcgtggtaccggaatgggaggtggcaatgatgagcgtgaacaaacccttaaccaattgttgatagagatggatgggtttgatggtaatgagaatattattgtcattgctgcaaccaaccgtagtgatattcttgatccagcccttctacgtccgggacgttttgaccgtaaagttcttgttggacgccccgatgtaaaaggtcgtgaagccatcttgaaagtacatgctaagaataaaccattggctgaagatgtcaaccttaaggttgttgctcagcaaactccaggttttgttggagctgacttagagaatgtcttgaatgaggctgctttagttgcggcacgacgtggtaagactaagattgatgctagtgatattgatgaagcagaggatcgtgtcattgctggaccatctaagaaagatcgccaagtttcggaacatgaacgtaaagttgttgcctaccatgaggctggtcacacgattgttggattaacactctctagtgctcgtgatgttcacaaagttactattgttccccgaggtcgtgcgggtggctatatgatttctttaccaaaagaagatcaaatgttgtcatctaaagacgaactgaaagaacaattagcgggattgatgggaggacgcgttgcagaagaaattatttttaatcttcaaacttctggtgcttccaacgactttgagcaggcaacacagttagcacgtgcaatggttactgaatatggtatgagtgaaaaacttggaccagtccaatatgaggggaaccacgcgatgaacccaggtcaattcacgccagacaagtcttactcagctcatacagctcaactcatcgacgaagaaattcgatcacttttagttgaggcacacgacagagcagctgaaattatcaatgcaaaccgagacacacatgctcttattgcagaagctttgcttaagtatgaaacacttgacgctgcacaaattaaatcaatttatgagactggtaagatgccggtagattcagaagaagataaccatgctttatcctttgatgaagttaaaaagagacttgaaaacaaaaataaagatgaagaagagtag

SEQ ID NO: 10 - protein, 655 aa, synthetic construct
MNNKNNNSFLRNAFLYIVMIIAVVTAVQYFVGGSQSPSQQLSYTQLIKKIEDGKVKSITYQPNGSIIEVKGDYKKAEKVDNDLSLPFLGGASSETQRFTSTVLHNETIIQALQSASEKANVNIKMIRESSSGTWISYLFSYLPLIGIAIFFIFMMNQGGNRGAMNFGRNRAKAQSKENIKVRFTDVAGAEEEKEELVEIVDFLKNPRKYKALGARIPKGVLLEGPPGTGKTLLAKAVAGEADVPFFSISGSDFVEMFVGVGASRVRSLFEDAKKAERAIIFIDEIDAVGRRRGTGMGGGNDEREQTLNQLLIEMDGFDGNENIIVIAATNRSDILDPALLRPGRFDRKVLVGRPDVKGREAILKVHAKNKPLAEDVNLKVVAQQTPGFVGADLENVLNEAALVAARRGKTKIDASDIDEAEDRVIAGPSKKDRQVSEHERKVVAYHEAGHTIVGLTLSSARDVHKVTIVPRGRAGGYMISLPKEDQMLSSKDELKEQLAGLMGGRVAEEIIFNLQTSGASNDFEQATQLARAMVTEYGMSEKLGPVQYEGNHAMNPGQFTPDKSYSAHTAQLIDEEIRSLLVEAHDRAAEIINANRDTHALIAEALLKYETLDAAQIKSIYETGKMPVDSEEDNHALSFDEVKKRLENKNKDEEE

SEQ ID NO: 11 - DNA, 1092 bp, synthetic construct
  source 1..1092: mol_type = other DNA
atgacagttacaggtattattgctgagttcaatccttttcataacggccacaagtatctcttggatcatgctgaaggcataaaaattgttgccatgtctgggaactttgttcagcgaggtgagccagctattgtggataagtggattagagctcagatggctttggaaaatggagcagatctcgtggttgagttgcccttctttacggcagtccagtctgccgattattttgcatctggggctgtcgatgtcttgtctcgactgggaattgatagtcttacttttggcacggaagaagtcctagactatcagactatagcagatgtctattctgaaaagtcagaggaaatggaagcctttgtggaaagtttaccgagtgacttgtcttatccacaaaagacgcaaaaaatgtgggaaaagtttgctggtgtggattttactggcaatacacccaaccatatcctaggattagcctatgccaaagcttgtgcgggtaagggcattacgttgaatcctatccagagacaaggggctggctatcattccttagacaaggaagtatctttcgcctcggcgaccagtctacgtctccacaaagaagatagtgattttgtggataagttcatgcccaatagcaaactttttcaaacttctccacaggtctcttgggataattatttccaattactggtttatcagattcttacaaaccccgatttgactagtgttttccaagtgaatgaagagattgctagccgtttaaaagctgctgtccgtgagatttcttctgtggaggagctggtggataaggtagcgaccaaacgctatacaaaagcacgggtacgtcgtattttgacctatatcttagtcggagctgtggataattctttgcctaaatctattcatgtccttggattttctcaaaaagggcagtttcatctcaaatctgtgaaaaagtctgtggatatagtggctcgtattggaaggaagccttgggatatgcttacacagcaagcggataatgtttatcaattaggtaaccccgagctttgcgaacaaaactttggacgtgtaccgattagagtaaaatag

SEQ ID NO: 12 - protein, 363 aa, synthetic construct
MTVTGIIAEFNPFHNGHKYLLDHAEGIKIVAMSGNFVQRGEPAIVDKWIRAQMALENGADLVVELPFFTAVQSADYFASGAVDVLSRLGIDSLTFGTEEVLDYQTIADVYSEKSEEMEAFVESLPSDLSYPQKTQKMWEKFAGVDFTGNTPNHILGLAYAKACAGKGITLNPIQRQGAGYHSLDKEVSFASATSLRLHKEDSDFVDKFMPNSKLFQTSPQVSWDNYFQLLVYQILTNPDLTSVFQVNEEIASRLKAAVREISSVEELVDKVATKRYTKARVRRILTYILVGAVDNSLPKSIHVLGFSQKGQFHLKSVKKSVDIVARIGRKPWDMLTQQADNVYQLGNPELCEQNFGRVPIRVK

SEQ ID NO: 13 - DNA, 852 bp, synthetic construct
  source 1..852: mol_type = other DNA
atgaaaaaaatagtgaaaacagttttgctagcctgtcttgttattttgcctttgtttgcgctatcagcttgtagctcgcgctcacattttgcaactcagaaagaccagtggcaaacttataccaaagaaaagaaaatcaaaattggttttgatgcaacctttgttcctatgggatatgaggaaaaggatggtagctatattggttttgatattgacttggctaatgcagtttttaaattatatggcatcgatgttgaatggcaagctattgactgggatatgaaagaaactgagttgaagaatgggacgattgatcttatttggaatggttactcggttacagatgaacgtaagcagtctgcagactttacggaaccttatatggtaaatgaacaagtactggtaaccaaaaagtcctcaggaattgattctgttgcaggaatggcaggtaagaccttaggtgctcaagctgggtcgtctggttatgatgcattcaatgcttctccaaaaattttgaaggatgtcgttgctaatcaaaaagttgtccaatattcgacttttacacaagctttgattgaccttaatagtggacgtattgatggcctattgattgaccgtgtgtacgctaattactacctagaaaaatcaggagttctggatcagtataatgttattcctgctggttacgagggagaaagctttgctgtaggtgctcgtaaggtagataagacactaataaaaaaaattaatcaaggatttgaaactctttataaaaatggagaattccaaaagatttctaacaagtggtttggtgaagatgtcgcaacagaccaagttaaagggaaaagataa

SEQ ID NO: 14 - protein, 283 aa, synthetic construct
MKKIVKTVLLACLVILPLFALSACSSRSHFATQKDQWQTYTKEKKIKIGFDATFVPMGYEEKDGSYIGFDIDLANAVFKLYGIDVEWQAIDWDMKETELKNGTIDLIWNGYSVTDERKQSADFTEPYMVNEQVLVTKKSSGIDSVAGMAGKTLGAQAGSSGYDAFNASPKILKDVVANQKVVQYSTFTQALIDLNSGRIDGLLIDRVYANYYLEKSGVLDQYNVIPAGYEGESFAVGARKVDKTLIKKINQGFETLYKNGEFQKISNKWFGEDVATDQVKGKR

SEQ ID NO: 15 - DNA, 801 bp, synthetic construct
  source 1..801: mol_type = other DNA
atggatctttatatcaaaaagaatattcagaatcaacacattttccagaattatgccagtgttgacgatattctttcctattctattgatgaggagttcattgatctcacgtcgtcattaaattactttattcccgataggactgttactagaaaagataagttagatatgatttcaggtcgagaacttgctaatagcaaccctgatactcttaaagaggagttaggtattgtgggcttagacctcttttttcatgccaatggtattaatgaaagtaatgttcataaaccttataaaacaaagtcacatggtttaggaaattcgcaaatattaccacgagactatgatcggcaagctgatattgaattgattctgaaagagatagcagaacaggtggctattagattaagacgagtccataagaaagcttgtcaagtttccattagcattggattttcaaagttagagggcaagcattcgctacaagcacatatgaagattgagcctgccaataatacaaaaatactaatagggcatgtcatttcactctttcggaagaaatatcaaggaggggctgttagaagtgtcagtgtttcttatgcaaattttgtagatgaaaagattcaaattctttctttatttgataatccagatgacattgataaagaagaaaggcttcagtcagctatagatagtattcgtcaggaatttggctttacgaccattcaaaaagcaacagctttacaggaggcatctaggagtattgctcaaagtaagcttattggtggacactcagcttga

SEQ ID NO: 16 - protein, 266 aa, synthetic construct
MDLYIKKNIQNQHIFQNYASVDDILSYSIDEEFIDLTSSLNYFIPDRTVTRKDKLDMISGRELANSNPDTLKEELGIVGLDLFFHANGINESNVHKPYKTKSHGLGNSQILPRDYDRQADIELILKEIAEQVAIRLRRVHKKACQVSISIGFSKLEGKHSLQAHMKIEPANNTKILIGHVISLFRKKYQGGAVRSVSVSYANFVDEKIQILSLFDNPDDIDKEERLQSAIDSIRQEFGFTTIQKATALQEASRSIAQSKLIGGHSA

SEQ ID NO: 17 - DNA, 1620 bp, synthetic construct
  source 1..1620: mol_type = other DNA
atggcaaaagatattaaattttcatcagatgcacgtgcagcaatggttcgtggtgttgatactttagctgataccgttaaggtaacgcttggccctaaaggtcgtaatgttgttttggaaaaggcttttggttcaccacttatcactaatgatggtgtgacaattgctaaagaaattgaacttgaagatcattttgaaaatatgggtgccaagctcgtcagcgaagtggcttcaaaaactaatgatattgccggtgacggaacaactacagccactgttttgacacaagctattgttcgtgaggggctaaaaaatgtaacagctggtgctaatccaatcggtatccgtcgtgggattgaagcagctgtagctgcagctgtggaagagcttaaagtcattgctcaaccagttgctaataaggaagctattgctcaagttgctgcggtttcatctcgctctgaaaaagttggtgaatacatttcagaagcaatggaacgcgttggtaatgatggtgttatcactattgaagagtctcgtggtatggagacagaactcgaagtggttgaaggtatgcaatttgaccgtggatatctttctcagtacatggttactgataatgaaaaaatggtcgcagatcttgaaaatccatatattcttgtgactgacaagaaaatttcaaatatccaagatattcttccactcttggaagaagtgcttaagaccagtcgtccacttttgattattgctgacgacgtggctggtgaagctcttccaacacttgttcttaacaaaattcgtggaaccttcaatgtcgttgctgttaaggcgcctggctttggtgaccgtcgtaaagcgatgttggaagatattgctgtattgactggagcaactgttattactgaggatcttggtcttgaacttaaggatgcaactatggaatcacttggtcaggcttcgaaagtaactgtggataaggatagtacagttatcgttgaaggagcaggaagcgcagaagcaattgctaaccgtgttaacctcattaaatctcaattggaaacaacaacatctgaatttgaccgtgaaaaacttcaagagcgtttggctaaattgtcaggtggtgttgcagttattaaggttggtgcagccacagaaacagctcttaaagaaatgaaacttcgtatcgaagatgctcttaatgctactcgtgccgctgtcgaagaaggtatagtagctggtggtggtacagccctagttaacgtcattgctaaggttgctgaacttgatcttgaaggtgacgatgctacaggacgtaatattgtcctacgtgccttggaagaacctgttcgtcaaattgcttataatgccggttatgaaggttcagttatcattgataaattgaagaatagcccagttggtacaggatttaacgctgccaatggcgaatgggtagatatggttgaatcaggaatcattgacccagttaaggtaacacgttcagcccttcaaaatgctgcttcagtagctagtcttatcttgacaacagaagcagtcgttgccgacaaacctgaacaaaaggctccagcagcgccagctacggatccaggaatgatgggttactaa

SEQ ID NO: 18 - protein, 539 aa, synthetic construct
MAKDIKFSSDARAAMVRGVDTLADTVKVTLGPKGRNVVLEKAFGSPLITNDGVTIAKEIELEDHFENMGAKLVSEVASKTNDIAGDGTTTATVLTQAIVREGLKNVTAGANPIGIRRGIEAAVAAAVEELKVIAQPVANKEAIAQVAAVSSRSEKVGEYISEAMERVGNDGVITIEESRGMETELEVVEGMQFDRGYLSQYMVTDNEKMVADLENPYILVTDKKISNIQDILPLLEEVLKTSRPLLIIADDVAGEALPTLVLNKIRGTFNVVAVKAPGFGDRRKAMLEDIAVLTGATVITEDLGLELKDATMESLGQASKVTVDKDSTVIVEGAGSAEAIANRVNLIKSQLETTTSEFDREKLQERLAKLSGGVAVIKVGAATETALKEMKLRIEDALNATRAAVEEGIVAGGGTALVNVIAKVAELDLEGDDATGRNIVLRALEEPVRQIAYNAGYEGSVIIDKLKNSPVGTGFNAANGEWVDMVESGIIDPVKVTRSALQNAASVASLILTTEAVVADKPEQKAPAAPATDPGMMGY

SEQ ID NO: 19 - DNA, 720 bp, synthetic construct
  source 1..720: mol_type = other DNA
atgtctgaagccataaaattatttcaatataatactttgggagccttgatggctgggctttatggaggtactttaaccgttggagagttgttagagcatggagacttgggtctcggaacattagattctatcgatggtgagttgattgttcttgacggtaaagcctaccaagctaaaggctctgaaggtaaagtcgaagtggtagaagtttctcctgatgaaaaagttccctatgcagcagtcgtacctcaccaagctgaagttatcttccgtcaacgttatgaaatgactgataaagaacttgaggatcgtattgaatcatactatgatggggtcaatcttttccgttcaatcaaaatcaaaggtcattttaaacacatgcacgtgcgcatgattccaaaatcaaatgcggacattaagtttgctgatgttgccactcgccaacctgagtatgaggtagatgatatttcaggtacaatcgttggtatctggacacctgaaatgttccacggtgtgagtgttgctggttaccatctccattttatttcagatgacttgacattcggtggtcacgttatagactttgttatcgagaatggtattattgaagttggaccagttgaccaactcgatcaacgcttcccagttcaagaccgtcaatatctctttgctaaattcaatgttgatgaaatgcgtaaagatattactaaggctgaataa

SEQ ID NO: 20 - protein, 239 aa, synthetic construct
MSEAIKLFQYNTLGALMAGLYGGTLTVGELLEHGDLGLGTLDSIDGELIVLDGKAYQAKGSEGKVEVVEVSPDEKVPYAAVVPHQAEVIFRQRYEMTDKELEDRIESYYDGVNLFRSIKIKGHFKHMHVRMIPKSNADIKFADVATRQPEYEVDDISGTIVGIWTPEMFHGVSVAGYHLHFISDDLTFGGHVIDFVIENGIIEVGPVDQLDQRFPVQDRQYLFAKFNVDEMRKDITKAE

SEQ ID NO: 21 - DNA, 2991 bp, synthetic construct
  source 1..2991: mol_type = other DNA
atgccagcagttactccagaacttgagatagaacgtcaattaatcgagcaattaacctctggtgagagccagtggacctatcgcccagacttaaaaaatgaaggtcaattatgggataacttttttgagaagttagcgcaaaataacgttgccctccttgctgaccatcccttgactgagcaggagaagcgccaaatcaagaaccagcttaacttcattaactattatgaagctgctaagtggttggctggtgaaaatggtattgccaaagtcgaggtacagcgtgaagatgctagccttgggactatccgactttctgtcatctggcgtgacaatatcgctgctggaaaatccagttacgaagtggtcaatcaagttgaacgtgacaaggcttattcacaagatcaggaccgtcgtttagatacgacgctgatgattaacggacttcctcttattcatatcgagctcaagagtcctcgtgtcgcctttttggatgcctttcatcaaatcaaaaagtatgaccgtgaaggaaagttacgtggcatctattctgcccttcagatgtttgttgtgacgaataaagtagacacacgctatatcgcagctgcacgagaagataaactcaataaacaattcttgacatcttgggtggataaagataataaacccatgaccagtctcatggactttgcccacgaagtcctgtctattccacgtgcccatcagatggtgatgcagtactccgttattgatgatagtaagaaagccctcatcctcttacgtccttatcaaattcatgccatagagtctgttcaggatgcctctcgccgtcaagaatctggctatatctggcataccacgggttctggtaagaccctgacctcctacaaggtggctcgcaacctcttgcagatcccagctatccaaaagaccatctttgtcgtcgaccgtagggaccttgaccagcagacgacctcgtcctttctctcttatgcggccaatgatgtcattgacatcgacgaaacggacaatactcatgacttggtcaagcgtttggcaggaaatgataagcgagtgattgtgacgactatccagaaaatcacaaccatgatgcgcaagtttggagagggtaaataccaaaaggattcggaaaaaatcaaagaccttcgtgtggctttcgttgtggatgaatgtcaccgtgcggtcacgcctcagactcaaaaggatatcaaaggctttttccacaattcgctctggtatggctttactggtacaccgattttcaaggaaaataaacgcaagcaattaggcgatttggcacaaactacccaccaacaatatggcgaacgcctacacgagtatacggtcaaagaagccatccatgacggggcagtcctaggattcaaggtagactatcgaaatactattatttcgccgattcctgaaaaagatcttcctgactctgtctatgaagataaagaacacatgctggaagtactggatgctatcctaaacaaaagttatcaacagttgggcttcccaaatggtgtgggtaagacctatgaggctatcttaaccgttaagtccattccgcaagctcaggcttattacaatctgctcaagagtatcaaggcaggacaggaacgtgtcaaggtgtcagagcgagctaagcgcgtgctacccgactttccaaagatgacggtcacttactctgtgtcggaaaatgaagaagagtctatcgactaccaagaccacatgaaacaggtcatggacgactacaatcaggagttcggtacccattttactatggctgatttgcgtggctttaatacggatatcaataatcgtttagccagaaagttagacaaatacattcctcgcaatgaacagttggatttggttatcgtcgttgaccgtcttttgaccggttttgatgccccctgtctatccactctcttcatcgaccgtaagcccatgcgtccacaggatttgattcaagcctttagtcgcaccaaccgcatctttgacagtaaaaagcgttatggtcatatcatcaccttccaaagaccagaggcctttaaagaggctgtggataacgcccttaaactctactcaaacggtggtgaaaatgatgtcactgccccagactgggcagaagaaaaagctaactttatccaagcctggatagactttcaagtaaaagtggaagatgtggaaaactatgtcatcacgattgagcaggctactagcccacaactccgtcgcattgctaaggcttaccaaacctttgacaagtatctggcatctatccgtgtctatagcgaatacgatgaagcggctatctatgcagaaactggcctatcagatgaaaagctcgaaacctatctaggaatctatcaaaatatcctagcagagctcaagagtcgagcagaagacgatggtgatgataatctctttgatatccactacgaactggaatcggtccaagtcgacgagattaactatgcctacatcctgacactgattcaaagtatgattcagcaggaagaggacagtccacaggccctcagtgacaaggatatcgaaacggttgataactatatccagagtctggaacgcaccaatcctaaactagccgaaatcatccgaaacttgtggaaagaagtccaagccaatcctaaagattaccaaggacaatctatcactggtgtcttggacactatgattgaagccattatcgacgaccatctcaaacactttgccagagaatggtatgtcggactggatgaacttcgttactatgtccaacactatcgtaagggtgccaagaagcaatctggagaaagccagctcaccaagagccaacgctacaaagactacaaagcagaaacagctgatgccctcaacccactcagctacaaaaaacatatcaaagaagcctacaccaaactcattgaagaggttattgaaccattgagagttgggaggtag

SEQ ID NO: 22 - protein, 996 aa, synthetic construct
MPAVTPELEIERQLIEQLTSGESQWTYRPDLKNEGQLWDNFFEKLAQNNVALLADHPLTEQEKRQIKNQLNFINYYEAAKWLAGENGIAKVEVQREDASLGTIRLSVIWRDNIAAGKSSYEVVNQVERDKAYSQDQDRRLDTTLMINGLPLIHIELKSPRVAFLDAFHQIKKYDREGKLRGIYSALQMFVVTNKVDTRYIAAAREDKLNKQFLTSWVDKDNKPMTSLMDFAHEVLSIPRAHQMVMQYSVIDDSKKALILLRPYQIHAIESVQDASRRQESGYIWHTTGSGKTLTSYKVARNLLQIPAIQKTIFVVDRRDLDQQTTSSFLSYAANDVIDIDETDNTHDLVKRLAGNDKRVIVTTIQKITTMMRKFGEGKYQKDSEKIKDLRVAFVVDECHRAVTPQTQKDIKGFFHNSLWYGFTGTPIFKENKRKQLGDLAQTTHQQYGERLHEYTVKEAIHDGAVLGFKVDYRNTIISPIPEKDLPDSVYEDKEHMLEVLDAILNKSYQQLGFPNGVGKTYEAILTVKSIPQAQAYYNLLKSIKAGQERVKVSERAKRVLPDFPKMTVTYSVSENEEESIDYQDHMKQVMDDYNQEFGTHFTMADLRGFNTDINNRLARKLDKYIPRNEQLDLVIVVDRLLTGFDAPCLSTLFIDRKPMRPQDLIQAFSRTNRIFDSKKRYGHIITFQRPEAFKEAVDNALKLYSNGGENDVTAPDWAEEKANFIQAWIDFQVKVEDVENYVITIEQATSPQLRRIAKAYQTFDKYLASIRVYSEYDEAAIYAETGLSDEKLETYLGIYQNILAELKSRAEDDGDDNLFDIHYELESVQVDEINYAYILTLIQSMIQQEEDSPQALSDKDIETVDNYIQSLERTNPKLAEIIRNLWKEVQANPKDYQGQSITGVLDTMIEAIIDDHLKHFAREWYVGLDELRYYVQHYRKGAKKQSGESQLTKSQRYKDYKAETADALNPLSYKKHIKEAYTKLIEEVIEPLRVGR

SEQ ID NO: 23 - DNA, 1383 bp, synthetic construct
  source 1..1383: mol_type = other DNA
atgagtaattacgctattatccttgctgcgggtaaaggaactcgcatgaaatcagatcttccaaaagtattacacaaggtttcaggcatcactatgcttgagcatgttttccgtgcagtttcagttatcgaaccagctaaaaatgttactgttattggtcataaggctgagcttgttcgtgaggttttggatggtcaatctgccttcactatgcaaactgaacagttggggactggtcacgcagtaatgatggctgaagaagagttggctggacttgaagggcaaacgcttgttatcgccggtgacacgcctttgattactggtgaaagccttaaaaaccttattgatttccacgttaatcataaaaatgttgccactatcttgactgcaactgccgataatccatttggttatggccgtatcatccgtaatgaaaatggtgaagtcactaagattgttgagcaaaaagatgcaaataagtttgaacaacaggttaaagaaattaatacagggacttatgtctttgacaataaacgtctcttcgaagcacttaaaaatattaatactaacaatgctcaaggagaatactatttgacagatgtcatctctattttccgtgaaaatggtgaaaaggtaggtgcgtatacccttcgtgactttgaagaaagtcttggagttaacgaccgtgttgccctagctacggcagaagatgttatgcgtcgtcgtattaacaagactcacatgattaatggtgttactttccaaaatccaaatgcgacatacattgatgtagatgtggaaattgcaccggacgttgtgattgaagctaatgttactcttaaaggaaaaactaaagtcggagcagagtcggtgttgactaatggaacttatattgttgattcaactatcggtgccaatactgttatcactaactcaatgattgagcattcagtggttgaaaaaggcgctacagttggtccatttgcccatattcgtccggattcaatgctcaaagaaggcgttcatattggtaacttcgtcgaggttaaggggtctactatcggtgaaaatactaaggccggtcacttgacctacattggtaatgcggaagttggttctgacgttaactttggtgcagggacgatcactgtaaactatgatggccaacataaattcaagacccaaattgctaacaacgcctttatcggaagcaattcaaccttgattgcaccgcttgaaattggggacaatgcacttacagcagctggttcaaccattacggataatgtcccagctgacagcgtagccattggacgcagccgtcaggtcaataaagaaggctacgctattaagaaacctcaccacccaagccaacaaaagtag

SEQ ID NO: 24 - protein, 460 aa, synthetic construct
MSNYAIILAAGKGTRMKSDLPKVLHKVSGITMLEHVFRAVSVIEPAKNVTVIGHKAELVREVLDGQSAFTMQTEQLGTGHAVMMAEEELAGLEGQTLVIAGDTPLITGESLKNLIDFHVNHKNVATILTATADNPFGYGRIIRNENGEVTKIVEQKDANKFEQQVKEINTGTYVFDNKRLFEALKNINTNNAQGEYYLTDVISIFRENGEKVGAYTLRDFEESLGVNDRVALATAEDVMRRRINKTHMINGVTFQNPNATYIDVDVEIAPDVVIEANVTLKGKTKVGAESVLTNGTYIVDSTIGANTVITNSMIEHSVVEKGATVGPFAHIRPDSMLKEGVHIGNFVEVKGSTIGENTKAGHLTYIGNAEVGSDVNFGAGTITVNYDGQHKFKTQIANNAFIGSNSTLIAPLEIGDNALTAAGSTITDNVPADSVAIGRSRQVNKEGYAIKKPHHPSQQK

SEQ ID NO: 25 - DNA, 933 bp, synthetic construct
  source 1..933: mol_type = other DNA
atgtctaaaatttttgtgtttggtcaccaaaatcctgattctgatgctattggttcgtcttatggatacgcttatttgaaacgtcaacttggtgttgaggctgaggctgttgctttgggtactccaaatgaagagactgcttttgtacttgactatttttctgtaaatgctcctcgtgttgttgagtcagcacggtctgagggggtaaatcaagttattcttactgaccataatgagtttcaacaatctatttcagatattaaggatgtagaagttattgaagtagtggatcaccaccgtgtggctaactttgagacggctaatccattaatgatgcgtttagaacctgtaggttcagcttcttcgattgtttatcgcatgtttaaagaaaataatgttgaaattcctaaagatgtagctggtttgcttttgtcaggtttgatttctgatactcttttacttaaatcaccaacgacacatgcttcagatcctgcagtagctgaagaacttgctcgacttgctggagtcaatttagaagaatatggtcttgccatgcttaaggctggtactaatctttcttctaaatcagctgaagaactcattgatattgatgcaaaaacatttgaattgaatgggaatcaagttcgcgttgctcaggtgaatactgttgatatttctgatgttctttcacgtcaagcggaaattgaagaagctatcaatagctcaattaaaagtaacgggtactctgactttgtgttaatgattactgatattcttaactcaaattcagaaattctcgcactcggatcaaatactgataaaattgaaaaagcttttaactttgttttagaaaataaccatgctttccttaaaggagcagtttctcgtaagaagcaagttgttcctcaattgactgaaagtttcaatgtgtaa

SEQ ID NO: 26 - protein, 310 aa, synthetic construct
MSKIFVFGHQNPDSDAIGSSYGYAYLKRQLGVEAEAVALGTPNEETAFVLDYFSVNAPRVVESARSEGVNQVILTDHNEFQQSISDIKDVEVIEVVDHHRVANFETANPLMMRLEPVGSASSIVYRMFKENNVEIPKDVAGLLLSGLISDTLLLKSPTTHASDPAVAEELARLAGVNLEEYGLAMLKAGTNLSSKSAEELIDIDAKTFELNGNQVRVAQVNTVDISDVLSRQAEIEEAINSSIKSNGYSDFVLMITDILNSNSEILALGSNTDKIEKAFNFVLENNHAFLKGAVSRKKQVVPQLTESFNV

SEQ ID NO: 27 - DNA, 1320 bp, synthetic construct
  source 1..1320: mol_type = other DNA
atgatacctaaagaatattatggacgactatttacgaaagaacagttaccagtggattatctctcagaggctgtaaaattagaaagtatgataaaggttgataaaaaacttagatgtaaaagatgttatagtcgaatagaggaagattggcaattaccgaatggtcagtattattgtagagcgtgtattgtctttggtcgaaaccaagaaggaaaagaactctattactttccctcagaaaaatcagaagtagattttcctgtcttgaaatggtcaggaaaactgactccttatcaaaatgaggtctcggaaaagcttttaaagacttataaaaatcaaaaacacagtcttgttcatgcagtgactggtgctggcaagacagagatgatttataatattgttgcctatgttcttgaaaataaaaatcgtgtcgtcatcgcaagtccccgagttgatgtttgtcgagaattgtttctacgcatgcagaaagattttacttgtagtatttctctgcttcatgctgatagtgaaccatatgatggtagtccgctcgttatagctaccactcatcaattactaaaattttatcatagctttgacttgattattgttgacgaagttgatgcctttccatttgtagggaatgtcatgttaaatcatgctgttaaacaggcaaagacggaaacaggccggtatatttacttgacagcaacttctacattagctttagaagagcaagtgcgccttggagctatagaaaagcatcaccttgctagtcgtttccacggaaatcctctagtccttcctcgtttcttttggcaaggaaggttacaaaagtcgttgacgagcgagaagcttccaaggcctctaattcaccagattaagaagcagcgtaaatcaaattttcctctattaatctttttccccaatatagcattaggtgaaaagtttagtgttaccctaaaaaaatatctccctactgaaaacatagcctttgtttcatcaaaaagcgaggagcgttcaaccatcgtagagaaattccgaaaaaaagaattgacaatcttagtgacgacaactattctcgaacgtggtgttacctttccacaagtagatgtttttgtttgtatggcaaatcatcacttatatactagttcgagtcttattcagattggtggtagggtggggcgttcgcccgagagacctacagggaaactctatttctttcatgaaggattatctaaatcaatgttgcaatgtcggaaagaaataaatgcaatgaataaaaaaggagggtttgaaaatgaagtgtctactatgtaa

SEQ ID NO: 28 - protein, 439 aa, synthetic construct
MIPKEYYGRLFTKEQLPVDYLSEAVKLESMIKVDKKLRCKRCYSRIEEDWQLPNGQYYCRACIVFGRNQEGKELYYFPSEKSEVDFPVLKWSGKLTPYQNEVSEKLLKTYKNQKHSLVHAVTGAGKTEMIYNIVAYVLENKNRVVIASPRVDVCRELFLRMQKDFTCSISLLHADSEPYDGSPLVIATTHQLLKFYHSFDLIIVDEVDAFPFVGNVMLNHAVKQAKTETGRYIYLTATSTLALEEQVRLGAIEKHHLASRFHGNPLVLPRFFWQGRLQKSLTSEKLPRPLIHQIKKQRKSNFPLLIFFPNIALGEKFSVTLKKYLPTENIAFVSSKSEERSTIVEKFRKKELTILVTTTILERGVTFPQVDVFVCMANHHLYTSSSLIQIGGRVGRSPERPTGKLYFFHEGLSKSMLQCRKEINAMNKKGGFENEVSTM

SEQ ID NO: 29 - DNA, 1257 bp, synthetic construct
  source 1..1257: mol_type = other DNA
atgacaatttttgaagaactaaaagcacgtggcttgatttttcaaaccacagatgaagaagcccttgtaaaagcctttgaagaagggccagtctcattctacactggttatgatccaacagcagacagtctccaccttggacacctcgtcgctatccttactagtcgtcgccttcaattggctggccacaaaccttacgccctcgttgggggtgcgacaggtttgattggtgacccatcatttaatgatgctgagcgtagcctccaaaccaaagaaaccgttgaaggttgggtagaaaaaatccaaggacaactctctcgtttccttgattttgaaaatggtgacaacaaagccgttatggtcaacaattatgactggtttggttctgtcagcttcatcgatttcttgcgtgatgtcggtaaatacttcacagttaactacatgatgagcaaggaatctgttaagaaacgtatcgaaacaggtatctcttacactgagttcgcttaccaaattatgcaaggttatgacttctatgaactcaatgctaaatatggcgtaaccctacaaattggtggttctgaccaatggggtaacatgacagcaggtactgagctcttgcgtcgtaaagcggataaatctggacacgtaatcacagttccacttatcacagattcaactggtaaaaaattcggcaaatctgaaggcaacgctgtctggcttgacgctactaagacaactccttatgaaatgtaccaattctggcttaacgttatggacgatgatgccgttcgtttcttgaaaattttcactttcttgtcacttgaagaaatcgaagaaatcggtaaagaatttgaccaagctcgtcaccaacgtttagcacaaaaggttctggctcgcgaagttgttactttggttcacggtaaagaagcctatgaacaagctgttcacatcaccgaacaactcttcgctggtaaccttaaagccctctcagcacgtgaccttaaagttgccctcaatggtgtcccaacctatgagatttcagcagatgaaaatcttaacattgttgaacttctcgtcaatgctaaaatttcaccatctaaacgtcaagcacgtgaagacgtgcaaaacggtgctatctatatcaacggtgaacgcgtgcaagacttggattacactctttcagataccgataagattgacaatgaaatcactgttatccgtcgcggtaagaagaaaaactttgttttaacttactaa

SEQ ID NO: 30 - protein, 418 aa, synthetic construct
MTIFEELKARGLIFQTTDEEALVKAFEEGPVSFYTGYDPTADSLHLGHLVAILTSRRLQLAGHKPYALVGGATGLIGDPSFNDAERSLQTKETVEGWVEKIQGQLSRFLDFENGDNKAVMVNNYDWFGSVSFIDFLRDVGKYFTVNYMMSKESVKKRIETGISYTEFAYQIMQGYDFYELNAKYGVTLQIGGSDQWGNMTAGTELLRRKADKSGHVITVPLITDSTGKKFGKSEGNAVWLDATKTTPYEMYQFWLNVMDDDAVRFLKIFTFLSLEEIEEIGKEFDQARHQRLAQKVLAREVVTLVHGKEAYEQAVHITEQLFAGNLKALSARDLKVALNGVPTYEISADENLNIVELLVNAKISPSKRQAREDVQNGAIYINGERVQDLDYTLSDTDKIDNEITVIRRGKKKNFVLTY

SEQ ID NO: 31 - DNA, 921 bp, synthetic construct
  source 1..921: mol_type = other DNA
atgtacgatacaattgttattggagctggtcctgcggggatgactgcagcactttatgcagctcgtgccaaccttaaagtggcaactttggagcaaggtgctcctggtggacagatgaataacacatctgatattgaaaattatcctggttttgaaagtatttctggtccagaactgtccatgaagatgtttgaacccttggaaaaattgggcgttgaaaatctttatggtatcgtgtctggtatcgaagacaggggcaattataaagttgtcaaaactggtgacgaggagtaccaaaccaagacagttatcattgcgacgggtgctaaacaccgccacattggtgttgctggtgaagaagagtacaacagccgtggtgtctcttattgtgcggtatgtgatggtgccttcttccgcaatcaagacctcttggttgttggtggtggagactctgctgttgaagaaggaatttatttgacacgctttgccaatagcgtgaccattgttcaccgtcgtgacgaattgcgtgctcaaaaggtccttcaagcccgtgccttcgccaatgaaaaggtcaagtttatctgggattccgtcgttgaagaaatcaagggtgatgacatcaaggttcgcagtgttgatatcaagaatgtcaaaactggtgaggtgactaaccacgaatttggcggtgtctttgtttatgtcggacttgatcctgtttcagattacttgacaggcctcgacatcactgaccaaaatggttgggtaatcacagatgataagatggcgacaagtgttccaggtatctttgccattggtgatgtgcgtcagaaagaccttcgccaaattaccacagccgtaggggaaggtgccatagccggtattgaagcttataactacgtgactgcacttggtgataagtaa

SEQ ID NO: 32 - protein, 306 aa, synthetic construct
MYDTIVIGAGPAGMTAALYAARANLKVATLEQGAPGGQMNNTSDIENYPGFESISGPELSMKMFEPLEKLGVENLYGIVSGIEDRGNYKVVKTGDEEYQTKTVIIATGAKHRHIGVAGEEEYNSRGVSYCAVCDGAFFRNQDLLVVGGGDSAVEEGIYLTRFANSVTIVHRRDELRAQKVLQARAFANEKVKFIWDSVVEEIKGDDIKVRSVDIKNVKTGEVTNHEFGGVFVYVGLDPVSDYLTGLDITDQNGWVITDDKMATSVPGIFAIGDVRQKDLRQITTAVGEGAIAGIEAYNYVTALGDK

SEQ ID NO: 33 - DNA, 2361 bp, synthetic construct
  source 1..2361: mol_type = other DNA
atgaaatttgtgaaaaaacagttacaaggcttgactcaggtggaggtcaagcagcggatagatgacggtaaaacaaattattttaaagcaaaagctggctccagcaattgggatatttttaggcgtaatgtctttaactcctttaatatgcttaattttgcaatctttgtggccttgattgctgttcaggcttggtctaatctatttttcttcgggattattgtgcttaatgcctttacagggatgatgacggagttgcgtgcccgtcgcatggttgataagctcaatctcatgaatcaggatcagattcgtgtggtgcgtgatggacagcttgtttccatcgatcctaaggatattgtcttagatgatatcatgctcttgtctgcgggtgagcaggttcccagtgatgcagtggttgttgatggaatggctgagcttaacgaggctatgctgactggtgagagtgatcagattctaaagaaagatgggaaggagttgctctcaggttcttatcttgtgagtggtcaggtctatgctaaggtcattaacgtagccgaggacaattacgccaacaagcttatgttagaggccaagactcataagccaatcgtgtcctgtatcctttataacatggacaagattgctaagtttacgggaaagattgttataccatttgggcttgcactctttttcgaggcttacatgattaagcagctttctcttcaagagtcggtcgttaccagttcaacagcccttctgggaatgttgcctaagggaattacccttttgactattacatcacttttgacggctgtcattaagcttgggatgaaacacatccttgtgcaagagatgtactctgttgaaaccttggcccgtgtagatgtgctttgtttagataagacagggacgattacacaaggaaagatgacagttaaaggtctagagctactgtcagagcgttttacgaaagaggagttggagcgtttgcttgcggcttatatgcaacatagtaaggataaaaacgcaacagctcaggctattcgaaatgcttatgagggtctggaacatcattatcaggtaggtgatgttattccattttcaagcaatcgtaaatggggtgctgtgagcatcaatgggcttggaactctctttttgggagcacctgagatgcttcttaagaagaatcctaaagcagtcgataaggctcaggcgcgagggtctcgtgttttgattttggcttggagtcagtcaggtattgccacagagaacatgattttgccgaatgatatcgaggctttgaccttgcttgaaattgccgatcctattcgtgaggatgctgcagaaactctggaatacctgcgttcagaagatgtgacacttaagattatttctggtgacaatccagtgacggtttctcatattgctcatcaagctggttttgccgattatcaaagctatattgactgttctaagatcagcgatgaggaattggaggctttggctgaggatactgcgatttttggtcgtgtatctccgcatcagaaaaagttattaatccaaacccttaatgccaatgggcataccacggctatgactggtgacggtgtcaatgatatcttggcactgcgtgaggcggattgttcgattgttatggcagagggagatccggctacacgccagattgctaacttggtgctaatggattctgagtttaaggacattcctgaaattctatttgagggacgccgtgtggttaataatattgcccacattgcacctattttcctcattaaaactgtttattccttccttttggggcttatctgtattggcagtattgtttttggaaaggcggagtacctcttggtctttccattcattcaggttcagatgaccttgacaggtcagtttatcgagggccttccaccatttgttttgaccttcgaacgtaatattcgtcctgttgagaaacatttcctcagacgttcgcttcagttggctattcctaatgccttgatgctggttatcagtgttcttattttccatctgtctcaggtctatcttggtatgagtagtacagacatgctgacgctttcttattatatgatgggatcaacgggtgttcttgccgttattcgtgcttgtatccccttaaatatggggcgtgtggccttgattatctactcagtctttggcttcttaatcagttcctactatcttcgagatgtgattgcaatctcaacacttaatagctacacattgccaatctatctggtcgcaatggctatctgtacaccgcttttcttctggattagctataaacaaggtgctttccaaaaagcacaaagcaaaacttaa

SEQ ID NO: 34 - protein, 786 aa, synthetic construct
MKFVKKQLQGLTQVEVKQRIDDGKTNYFKAKAGSSNWDIFRRNVFNSFNMLNFAIFVALIAVQAWSNLFFFGIIVLNAFTGMMTELRARRMVDKLNLMNQDQIRVVRDGQLVSIDPKDIVLDDIMLLSAGEQVPSDAVVVDGMAELNEAMLTGESDQILKKDGKELLSGSYLVSGQVYAKVINVAEDNYANKLMLEAKTHKPIVSCILYNMDKIAKFTGKIVIPFGLALFFEAYMIKQLSLQESVVTSSTALLGMLPKGITLLTITSLLTAVIKLGMKHILVQEMYSVETLARVDVLCLDKTGTITQGKMTVKGLELLSERFTKEELERLLAAYMQHSKDKNATAQAIRNAYEGLEHHYQVGDVIPFSSNRKWGAVSINGLGTLFLGAPEMLLKKNPKAVDKAQARGSRVLILAWSQSGIATENMILPNDIEALTLLEIADPIREDAAETLEYLRSEDVTLKIISGDNPVTVSHIAHQAGFADYQSYIDCSKISDEELEALAEDTAIFGRVSPHQKKLLIQTLNANGHTTAMTGDGVNDILALREADCSIVMAEGDPATRQIANLVLMDSEFKDIPEILFEGRRVVNNIAHIAPIFLIKTVYSFLLGLICIGSIVFGKAEYLLVFPFIQVQMTLTGQFIEGLPPFVLTFERNIRPVEKHFLRRSLQLAIPNALMLVISVLIFHLSQVYLGMSSTDMLTLSYYMMGSTGVLAVIRACIPLNMGRVALIIYSVFGFLISSYYLRDVIAISTLNSYTLPIYLVAMAICTPLFFWISYKQGAFQKAQSKT

SEQ ID NO: 35 - DNA, 3726 bp, synthetic construct
  source 1..3726: mol_type = other DNA
atggataaacgtattttcgttgagaaaaaagctgattttcgtgtgaaatctcgctctttattaaaagaattacagcataatattcagttgaaaaccttgaatgatcttcgtattgtacaagtttacgatgtctttaatttggcagaggatttgtttgcccgtgcggaaaaatatatcttttctgaacaagtgaccgatactgttttggatgaagctacagttaagactgaccttgagaagtatgctttctttgctatcgaaagcttgcctggtcaatttgatcaacgtgcagcatcttcacaagaagctttgcttttgcttggtagctcaagtgatgtaacggttaatactgctcaattgtacttggttaacaaggacattgctgctaatgaattggaagctgttaaaaattatctattgaacccagtggactctcgtttcaaagatatcactgttggtattgctaaacaggatttctctgagtctgacaaaacgattccaaacttggatttctttgaaacttatacagcagaagattttgcacagtataaggcagatcaaggattggccatggaagtggatgacctgctcttcattcaagattacttcaagtcaattggacgtgtgccaactgagactgaacttaaggttttggatacttactggtctgaccactgccgccacacaactttcgagactgagttgaaaaacatcgacttctcagcttctaaattccaaaaacaattgcaagcaacttatgacaaatatattgccatgcgtgatgagttgggacgtgcagaaaagcctcaaaccttaatggatatggcgactatttttggtcgttatgagcgtgccaatggccgtttggatgacatggaagtttctgacgaaatcaatgcctgctcagttgaaattgaagtagatgtcaatggtgtcaaagaaccatggcttcttatgttcaagaacgaaactcataaccacccaacggaaatcgaaccatttggtggagcggctacttgtatcggtggtgccattcgtgacccattgtcaggtcgttcatacgtttaccaagctatgcgtatttcaggtgccggtgatattacaacaccaatcgctgaaacacgtgctggaaaattgccacaacaagtcatttctaaaaaagcggctcatggttattcttcatatggtaaccaaattggtcttgcaacaacttatgttcgtgaatacttccacccaggtttcgttgccaagcgtatggagctaggtgcagttgtcggtgcagctcctaaggagaatgttgtccgtgaaaaacctgaagctggagatgtggttatcttgctcggtggtaagactggacgcgacggtatcggtggtgcgacaggttcttcaaaagttcaaacggttgaatcagttgaaacagctggtgctgaggttcaaaaaggtaatgctatcgaagaacgtaagattcaacgtcttttccgcaatggtgaagtaacacgtcttatcaagaaatccaatgactttggtgctggtggtgtctgtgtggctatcggtgaattggcagatggtcttgaaattgatctagacaaagtgccattgaaataccaaggtttgaacggtaccgaaattgccatctctgaatctcaagaacgtatggctgtggtggttcgtccagaagatgtagatatcttcgttgctgaatgtaacaaagaaaatattgatgcagttgtcgttgcaaccgtgactgaaaaaccaaatctcgtcatgcactggaatggtaaaacaattgttgatttggaacgtcgtttccttgatacaaatggtgtgcgtacagtagtagatgttaaggtggttgacaaggatgtcaagcttccagaagaacgcaaaacctctgctgaaacccttgaagcagatacgcttgaagttttggctgaccttaaccatgcaagtcaaaaaggtttacaaactatgtttgatagctcggttggtcgttcaacagtcaatcacccacttggtggtcgataccaaattactccaactgaagcttctgttcagaaattgccagtccaatatggcgtgacaacaactgcctctgtcatagcccaaggatttaacccatacatcgcagaatggtctccatatcatggtgctgcatatgcagtcatcgaagcaacagctcgtttggttgctgttggtgcaaattggtctaaggctcgtttctcttaccaagaatacttcgagcgtatggataaaaaagcagagcgttttggtcaaccagtatcagctcttcttggatcaatcgaagctcagattcaacttggtttgccatctattggtggtaaggactctatgtcaggcacctttgaagaattgacagtaccaccaaccttggttgcttttggggtgacaacagcagacagtcgtaaggtcctttctccagaatttaaggctgctggtgaaaatatctactacatcccaggtcaagctttggcccaagagattgattttgatctcatcaagtctaactttgctcagtttgaagctatccaagctgatcacaaagtgacagctgcatcagctgtcaaatatggtggtgtcattgaagctcttgcacttgcaacgtttgggaaccatatcggggcgactgtaacccttgaaaatcttgagactgccttaacagctcaattgggaggattcatcttcacatctccggaagagattgcaagtgttggcaagattggacaaacagtagctgactttacacttgctgtcaacggtgtaacgcttgatggatataaacttgacagtgcctttcaaggtaaattggaagaggtttacccaactgaatttgcacaagcaactgagttagaagaagtacctgctgtgacatcagatactgttataaaagctaaagaagtagttgagaaaccagtggtttacatcccagtattcccaggtactaactctgagtatgactctgctaaggcctttgaaaaagaaggtgccaaagtcaacttagtaccatttgtaacattgaatgaagaagcgattgtcaagtctgttgacaccatggttgacaatattgaaaaagctaacattatcttccttgcaggtggcttctcagcagcggatgaaccagatggagcagctaaatttatcgttaacatcttgctcaatgaaaaagtacgtgcagctattgatagcttcatcgaacgtggtggtttaatcatcggtatctgtaacgggttccaagctcttgtcaaatcaggtctccttccatacggtaactttgaagatgcaagcagtactagtccaaccctcttctacaatgatgccaaccaacacgtggctaagatggttgaaacacgtattgccaatactaactcaccttggcttgctggagtgaaagttggtgacatccataccatcccagtatcacacggtgaaggtaaatttgtcgtgacgactgaagaatttgcggagcttcgcgataatggtcaaatctttagccaatatgttgacttcgaaggcaaacctagcatggattctaaatataatccaaatggttctgtcaatgctattgaaggtatcacaagcaagaacggtcaaattattggtaagatgggacactcagaacgtttcgaagatggtctcttcaaaaatattccaggaaataaagaacagcacctatttgcgtcagcggttaaatacttcactggaaaataa

SEQ ID NO: 36 - protein, 1241 aa, synthetic construct
MDKRIFVEKKADFRVKSRSLLKELQHNIQLKTLNDLRIVQVYDVFNLAEDLFARAEKYIFSEQVTDTVLDEATVKTDLEKYAFFAIESLPGQFDQRAASSQEALLLLGSSSDVTVNTAQLYLVNKDIAANELEAVKNYLLNPVDSRFKDITVGIAKQDFSESDKTIPNLDFFETYTAEDFAQYKADQGLAMEVDDLLFIQDYFKSIGRVPTETELKVLDTYWSDHCRHTTFETELKNIDFSASKFQKQLQATYDKYIAMRDELGRAEKPQTLMDMATIFGRYERANGRLDDMEVSDEINACSVEIEVDVNGVKEPWLLMFKNETHNHPTEIEPFGGAATCIGGAIRDPLSGRSYVYQAMRISGAGDITTPIAETRAGKLPQQVISKKAAHGYSSYGNQIGLATTYVREYFHPGFVAKRMELGAVVGAAPKENVVREKPEAGDVVILLGGKTGRDGIGGATGSSKVQTVESVETAGAEVQKGNAIEERKIQRLFRNGEVTRLIKKSNDFGAGGVCVAIGELADGLEIDLDKVPLKYQGLNGTEIAISESQERMAVVVRPEDVDIFVAECNKENIDAVVVATVTEKPNLVMHWNGKTIVDLERRFLDTNGVRTVVDVKVVDKDVKLPEERKTSAETLEADTLEVLADLNHASQKGLQTMFDSSVGRSTVNHPLGGRYQITPTEASVQKLPVQYGVTTTASVIAQGFNPYIAEWSPYHGAAYAVIEATARLVAVGANWSKARFSYQEYFERMDKKAERFGQPVSALLGSIEAQIQLGLPSIGGKDSMSGTFEELTVPPTLVAFGVTTADSRKVLSPEFKAAGENIYYIPGQALAQEIDFDLIKSNFAQFEAIQADHKVTAASAVKYGGVIEALALATFGNHIGATVTLENLETALTAQLGGFIFTSPEEIASVGKIGQTVADFTLAVNGVTLDGYKLDSAFQGKLEEVYPTEFAQATELEEVPAVTSDTVIKAKEVVEKPVVYIPVFPGTNSEYDSAKAFEKEGAKVNLVPFVTLNEEAIVKSVDTMVDNIEKANIIFLAGGFSAADEPDGAAKFIVNILLNEKVRAAIDSFIERGGLIIGICNGFQALVKSGLLPYGNFEDASSTSPTLFYNDANQHVAKMVETRIANTNSPWLAGVKVGDIHTIPVSHGEGKFVVTTEEFAELRDNGQIFSQYVDFEGKPSMDSKYNPNGSVNAIEGITSKNGQIIGKMGHSERFEDGLFKNIPGNKEQHLFASAVKYFTGK

SEQ ID NO: 37 - DNA, 1344 bp, synthetic construct
  source 1..1344: mol_type = other DNA
gtgataaaaatttatgatacaatgacacgttcgctccgtgatttcgtacctttgacagagaacactgtcaatatgtatgtttgcggaccaaccgtctacaattatattcatatcggaaatgcccgttcaacagtagcttttgataccattcgtcgttattttgagtatcgtggttacactgttaactacatttctaactttacagatgtagatgataagattatcaaggcggccaatgaagttggtatttcgaccaaagaattatcagacaagtttatcacagcctttatggaagataccgcacaacttggaatcaaaccagcgacacaaaatcctcgtgttattaactacatggatgaaatcattgcatttgtatcaatcttgattgataaaggttttgcttatgtttctgaaggtgatgtatacttccgagtaactaagtcaaacaattatgctaaattagccaacaagaccttggaagatcttgaaattggtgctagcggacgtacggatgctgagacagatcgtaaggaagatcctctggattttgccctttggaaagctgccaaagaaggagagatttcttgggaaagtccttggggacctggacgtccaggctggcacatcgaatgttctgttatggcaactgaaatccttggggatactatcgatatccatggtggaggtgtggaccttgagttcccacaccatactaatgaaatcgcacaatcagaagccaagactggtaagacctttgctaattactggatgcataacggttttgtcaatgtggacaatgagaaaatgtcaaaatccttgggtaactttgtgactgttcatgatatgcttaagacggttgatgggcaagtgcttcgcttcttcctagcgacacaacagtatcgtaaaccaattaactttaccgagaaagctatccatgatgctgaagtcaatctcaagtatttgaaaaatacgcacagtctaccactaacagaagaggtaagccaagccgaattgcaagcttatcttgatgctttccaggcggctatggatgatgactttaatacggcaaatggtgtgactgttctgtttgatatggctaaatggattaattcaggcaactatgatgaaactgttaaaaatgcttttgaaaaaattctacaagtcttcggtattgttttcgaagaagaaactcttgatgaagatatcgaaaaactcattgaagagcgtcaagcagctcgtgccaacaaggactttgcgacagcagatcgtattcgtgatgagttagctgctcaaggtatcaaacttcttgataccaaggagggcgtgaggtggacacgtgactaa

SEQ ID NO: 38 - protein, 447 aa, synthetic construct
VIKIYDTMTRSLRDFVPLTENTVNMYVCGPTVYNYIHIGNARSTVAFDTIRRYFEYRGYTVNYISNFTDVDDKIIKAANEVGISTKELSDKFITAFMEDTAQLGIKPATQNPRVINYMDEIIAFVSILIDKGFAYVSEGDVYFRVTKSNNYAKLANKTLEDLEIGASGRTDAETDRKEDPLDFALWKAAKEGEISWESPWGPGRPGWHIECSVMATEILGDTIDIHGGGVDLEFPHHTNEIAQSEAKTGKTFANYWMHNGFVNVDNEKMSKSLGNFVTVHDMLKTVDGQVLRFFLATQQYRKPINFTEKAIHDAEVNLKYLKNTHSLPLTEEVSQAELQAYLDAFQAAMDDDFNTANGVTVLFDMAKWINSGNYDETVKNAFEKILQVFGIVFEEETLDEDIEKLIEERQAARANKDFATADRIRDELAAQGIKLLDTKEGVRWTRD

SEQ ID NO: 39 - DNA, 1176 bp, synthetic construct
  source 1..1176: mol_type = other DNA
atggcagaaagaatccttatgactaatggcaagctcaaagttactgataatccgattattcctttcattgaaggagatggtgttggacgtgatatttggaagaatgctcaagcaatttttgataaagcggtagaagtatcctatggaggacagcgtcgtattgagtggcaagagcttttggcaggtaaaaaggcttttgataaaacaggtgagtggttgccgcaagaaactttagaggctattcgagaaagcctggtagctattaaggggccacttgaaacacctgtcggaggaggaattcgttctttaaatgtagctttacgtcaagaacttgatctctacgcttgtgttcgaccggttcggtatttcgaaggagtagcaagtccgcttaaggaaccagaaaaaacagacattactatttttcgtgagaatactgaggatatttatgcaggaattgaatgggaagcaggaacagtagatgtagagcgcgttattgctttttttcaaacagaaatgaatgtcgataaaattcgctttcccaagtcaagtagtattggaattaagcctatttcaatagaaggaagtaaacgattgattcgttcagctattgactatgctcttaagaacaatctaaagaaagtgacattggtgcataaagggaatattcaaaaattcacagaaggtggctttcgtaaatggggctatgaagtggctgaggaagaatataaggaagaaatgcttgctggccgtctagaagttaatgatataatcgctgataattttctacaacagattcttttgaatcccgaaaaatttgatgttgttgcattaaccaacttgaatggcgattatgcaagtgatgctctagctgcccaggttggtggtatcggaatctcccctggagctaatattaactatcaaacaggccatgctatctttgaagctactcatggaacggctccagatattgcggatcaagatagggctaatccttgctctattctcttatctggctgtatgcttttagaatatatcggttggtctgaagcagctgaactcattataagtgctattgaaaaaaactttaaatcagggatttttacagctgacctagcctttggaaaacgagcctattcaaccagtggtttcagcaatcaaattatctcaattatatga

SEQ ID NO: 40 - protein, 391 aa, synthetic construct
MAERILMTNGKLKVTDNPIIPFIEGDGVGRDIWKNAQAIFDKAVEVSYGGQRRIEWQELLAGKKAFDKTGEWLPQETLEAIRESLVAIKGPLETPVGGGIRSLNVALRQELDLYACVRPVRYFEGVASPLKEPEKTDITIFRENTEDIYAGIEWEAGTVDVERVIAFFQTEMNVDKIRFPKSSSIGIKPISIEGSKRLIRSAIDYALKNNLKKVTLVHKGNIQKFTEGGFRKWGYEVAEEEYKEEMLAGRLEVNDIIADNFLQQILLNPEKFDVVALTNLNGDYASDALAAQVGGIGISPGANINYQTGHAIFEATHGTAPDIADQDRANPCSILLSGCMLLEYIGWSEAAELIISAIEKNFKSGIFTADLAFGKRAYSTSGFSNQIISII

SEQ ID NO: 41 - DNA, 1176 bp, synthetic construct; identical to SEQ ID NO: 39
  source 1..1176: mol_type = other DNA

SEQ ID NO: 42 - protein, 252 aa, synthetic construct
MTEPIISINDLSVYFNKKKAINNVTMDFYPNEITALIGPSGSGKSTLLRSINRMGDLNPECTVTGAVSYNGHNVYSPRTDTVELRKEIGMVFQQPNPFPMSIYENVVYGLRINGIKDKAVLDKAVEESLKGASIWEEVKDRLHDSALGLSGGQQQRVCVARVLATSPKVILLDEPTSALDPISAGKIEETLYKLKDQYTLLLVTRSMQQASRISQRTAFFLDGELIEYSSTKDMFLNPQHKETEDYITGKLG

SEQ ID NO: 43 - DNA, 768 bp, synthetic construct
  source 1..768: mol_type = other DNA
atggcgacaattaaagaagtcaaggaacaattggctattttgagggatcttgacgaccctagatgggcaagttttgaggaagatagtcggactggtgtacaagctgccattaggaaacgccgtaaggctatcctcgctgaacttgctgaggaagaacgtttagaaacgcttcttaattatgagaaatcactctatgctcgaggaatagaactcatcgcaggtgtcgatgaagttggacgtggtcctttagctgggccagttgtagcggctgcggtcattttaccaaagctttgcaaaataaaaggtctaaatgacagtaaaaagattccaaaatctaaacatgaagctatctataatcaggtaatgaaggaggcagtagctgtcggtattggtattaaggataattatgtgattgatgacgtcaatatttatgaagctaccaagttagccatgatagaagctattgaaaaactcaatccccaacctgaacatttgctaattgatgccatgaatttggacttacctatagaacaaacctcaatcataaaaggggatgctaattctctatctattgcagcagcttctatcgcagcaaaagtaactcgggataagatgatggctgattatgagcaggagtttcctggatacgcctttgccaaaaatgcaggctatggcaccaaagaacatttgtctggcatagataagtttggtgtcacgccaattcatagaagaagcttcgaaccaataaagtcaataataaaaaagtag

SEQ ID NO: 44 - protein, 255 aa, synthetic construct
MATIKEVKEQLAILRDLDDPRWASFEEDSRTGVQAAIRKRRKAILAELAEEERLETLLNYEKSLYARGIELIAGVDEVGRGPLAGPVVAAAVILPKLCKIKGLNDSKKIPKSKHEAIYNQVMKEAVAVGIGIKDNYVIDDVNIYEATKLAMIEAIEKLNPQPEHLLIDAMNLDLPIEQTSIIKGDANSLSIAAASIAAKVTRDKMMADYEQEFPGYAFAKNAGYGTKEHLSGIDKFGVTPIHRRSFEPIKSIIKK

SEQ ID NO: 45 - DNA, 213 bp, synthetic construct
  source 1..213: mol_type = other DNA
atgcgaccgcttgaacagattgaaaagttgattgcagaagcaaaaaaatatgaatttgcaagtgtttgtgttaatccaacttgggtaaagactgctgctgatttgctaaaagatagttccattaaggtttgtacagtaattggattcccactaggagcaacaacatcgtctgttaaagcgtttgaaacacgagatgcaatttcaatggtgtag

SEQ ID NO: 46 - protein, 70 aa, synthetic construct
MRPLEQIEKLIAEAKKYEFASVCVNPTWVKTAADLLKDSSIKVCTVIGFPLGATTSSVKAFETRDAISMV

SEQ ID NO: 47 - DNA, 741 bp, synthetic construct
  source 1..741: mol_type = other DNA
atgactgagacgattttagaaatcaaaaacctcaaaaaatcatacggaaaaaacgaagttctcaaagatatctccctcagtgtcaaaaagggggaggttatctcaattattggttcttcaggttcaggaaaatcaaccttccttcgctctattaacctactcgaatcaccttcaggtggtgaaatcctctatcatggggataacgtccttgaaaaaggttatgatctcactacctatcgtgagagactcggtatggtgttccaatccttcaacctctttgaaaaccttaatgtcttggaaaatgctatcgtagctcaaacaactgttctcaaacgtgaccgtaaagaagccgagagtatcgccaaagctaacttagagaaagttggtatgggggaacaatactggaaagctaaaccaaaacaactttcagggggccaaaaacaacgtgtggcgattgctcgtgccctctctgttgacccagaagccatcctctttgacgagccaacttcagcccttgacccagaaatggtaggggaagtactcaagaccatgcaggagctatctggaaccgggttgaccatgattatcgttacccatgaaatggagttcgcacgcgatgtttcagaccgtgtcatcttcatggataaaggggtaatcgctgaacaaggtagcccagaacaaatctttgaaaatcctaaagaagaacgtaccaaggaattcctcaaacgtttcttgggttaa

SEQ ID NO: 48 - protein, 246 aa, unidentified
MTETILEIKNLKKSYGKNEVLKDISLSVKKGEVISIIGSSGSGKSTFLRSINLLESPSGGEILYHGDNVLEKGYDLTTYRERLGMVFQSFNLFENLNVLENAIVAQTTVLKRDRKEAESIAKANLEKVGMGEQYWKAKPKQLSGGQKQRVAIARALSVDPEAILFDEPTSALDPEMVGEVLKTMQELSGTGLTMIIVTHEMEFARDVSDRVIFMDKGVIAEQGSPEQIFENPKEERTKEFLKRFLG

SEQ ID NO: 49 - DNA, 585 bp, synthetic construct
  source 1..585: mol_type = other DNA
gtgagacgacgttttaaactaaaagcctttataatcttagttgctgttttcgctttagggattctcttaccgctgattcttcatgcatctacagctgataatgctcgtacggtaaaggttgcctatacgcagaaagggtttattgaaaacttagcgccaacagcacaaaaaatgtctaaaaactatggtgtccctgcgtcgattctccttagccaagccgcatatgagtcgaactatggtagtagtctcttatctgttaaataccataatatctacagtctccctgctcggccaggacaagaacatatctatctaaaggataatgtttatagtaaaggcaaatggcaatatcaaaaagtagattttgcagtttttagagactggtcaagttctatgtcatcttatctagaagaactacgccaaggtagatggggagaatcaacctataaagaggtggcaggaactacgagttataaagttgcggcagaaaagctacaagctgcaggttttaatagtgatcctgattacgctaaacatcttatttctattattgagacctataatttggtaaaatatgatcgctga

SEQ ID NO: 50 - protein, 194 aa, synthetic construct
VRRRFKLKAFIILVAVFALGILLPLILHASTADNARTVKVAYTQKGFIENLAPTAQKMSKNYGVPASILLSQAAYESNYGSSLLSVKYHNIYSLPARPGQEHIYLKDNVYSKGKWQYQKVDFAVFRDWSSSMSSYLEELRQGRWGESTYKEVAGTTSYKVAAEKLQAAGFNSDPDYAKHLISIIETYNLVKYDR

SEQ ID NO: 51 - DNA, 360 bp, synthetic construct
  source 1..360: mol_type = other DNA
atgaacactcctcatcaggatttccaaaaagccaaagaagagttaattgacctcctcaaacaccatgaggcggttcttgcttttcaagaggctgaggaatctattggacagattcctcagatcagcgatctggctggacagatgaaggcctatcaacaagaagcggttctttttcaaaagatcgagaaacaacgtgcctatgaggaggctggtgagcaggcagatttaattcagcatgagttggaaaatttgcccattgttcaggattaccgtcaaaagatgcaagatgccagtgacttgattcaatatgtgaccaagtctatcgaagaaaggattaacgaggagttaagacatggctaa

SEQ ID NO: 52 - protein, 119 aa, synthetic construct
MNTPHQDFQKAKEELIDLLKHHEAVLAFQEAEESIGQIPQISDLAGQMKAYQQEAVLFQKIEKQRAYEEAGEQADLIQHELENLPIVQDYRQKMQDASDLIQYVTKSIEERINEELRHG

SEQ ID NO: 53 - DNA, 291 bp, synthetic construct
  source 1..291: mol_type = other DNA
atggctaaatacgaaattctttatatcattcgtccaaacattgaagaagaagctaaaaacgctttggtagcacgtttcgattctatcttgactgacaacggtgcaactgttgttgaatcaaaagattgggaaaaacgtcgtcttgcatacgaaatccaagatttccgtgaaggaatttaccacgtcgtaaacgttgaagcaaacgacgctacagctcttaatgagttcgaccgtctttcaaaaatcaacggcgatattcttcgtcacatgatcgttaaacttgacgcgtaa

SEQ ID NO: 54 - protein, 96 aa, synthetic construct
MAKYEILYIIRPNIEEEAKNALVARFDSILTDNGATVVESKDWEKRRLAYEIQDFREGIYHVVNVEANDATALNEFDRLSKINGDILRHMIVKLDA

SEQ ID NO: 55 - DNA, 3180 bp, synthetic construct
  source 1..3180: mol_type = other DNA
atgcctaaacgttctgatattaaaaaaattatggttatcgggtctggtcctattatcattggtcaggctgctgagttcgactacgctggtacgcaagcttgcttggctttgaaagaagaaggctacagtgttgttcttgttaactctaaccctgcgactatcatgactgataaggaaatcgcagacaaggtttacattgaacctattacgcttgagtttgtcacacgtattcttcgtaaagagcgtcctgatgccctcttgccaacacttggtggacagactggtttgaatatggccatggaattgtcaaaagctggtattcttgatgagcttggagtagagcttttgggaacaaaattgtctgccattgaccaagccgaagaccgtgaccttttcaaacaattgatggaagagttggaacaaccaattccagaatcagaaattgtaaatacggttgaagaagcagttgcctttgcgacagagattggctacccagttatcgttcgtccagcctttacccttggtggtactggtggtggtatgtgtgctaacgaagaagaactccgtgaaatcgctgaaaatggattgaaattgtcaccagtaacccaatgtttgattgagcgttcgatcgctggtttcaaagaaatcgaatacgaagttatgcgtgatgccgaagacaatgctcttgtggtatgtaacatggaaaactttgacccagttggtatccacacaggggactcaatcgtatttgccccaacgcaaaccttatcagatattgaaaaccaaatgcttcgtgatgccagcttgaagattatccgtgcccttaaaattgagggtggctgtaacgtccagttggcgcttgacccacatagcttcaagtactatgttatcgaagtaaaccctcgtgtatcacgttcatcagcccttgcctcaaaggccactggttatccaatcgctaaattggcggctaagattgctgttggtttgacacttgatgaaatgattaacccagtcactggtacaacttatgccatgtttgaaccagcccttgactatgtggttgctaagattccacgtttcccatttgataaatttgaacacggtgaacgtcgtctcggtactcagatgaaagcgacaggtgaagttatggctatcggtcgtaacattgaagagtcacttcttaaagcatgccgttcacttgaaatcggagtttaccacaatgaaatgtctgagcttgccgaagtaacagacgatgccttggttgaaaaagttgttaaagctcaagacgaccgcctcttctatatttctgaggccattcgtcgtggttacactattgaagaattatcagagttgactaagattgatatcttcttccttgataaattgctccacatcttcgaattggaacaagaattggctgctcacgtaggcgatgttgatgttttaaaagaagctaaacgtaacggtttctctgatcgtaagattgctgatctttggaatcaaacagctaaccaagtacgtgcgacacgtttggaaaataacattgttcctgtttacaagatggttgatacttgtgcggctgagtttgaatcatcaacaccatatttttactcaacttacgagtgggaaaatgagtcaatcaaatctgataaagaatcggtcatcgttcttggttcaggtcctatccgtatcggacaaggggttgagttcgactatgcgacagttcactctgtaaaagctatccaagctgctggctacgaagccatcatcatgaactctaaccctgagacagtttcaacagacttctcagtgtcagacaaactctactttgaaccattgaccttcgaagacgtgatgaacgttattgaattggaacaacctaagggtgtggtggttcagtttggtggacaaacagccatcaacttggcagagccattgtctaaagcaggtgtgaaaatcttgggtacacaggttgctgaccttgaccgtgcagaagaccgtgacctcttcgaacaggctcttaaagatcttgacattccacaaccaccaggacaaacagcgacaaatgaagaagaagcagttgaagcggctcgtaagattggtttcccagttcttgttcgtccatcatacgttttgggtggacgtgctatggaaattgttgaaaatgaagatgaccttcgttcttacatgcgcacagccgttaaggctagtccagaccacccagtccttgttgatagctatatcataggacgtgagtgtgaagtggatgccatctctgatggtaaggatgtcttaattccaggtattatggaacacatcgaacgtgcgggggttcactcaggggactcaatggcggtttacccaccacaaactctttctaagaaaatccaagagactatcgctgattacacgaaacgtttggctatcggtcttaactgtatcggtatgatgaacatccagttcgttattaaggacgaaacagtctatgttatcgaggttaacccacgtgccagccgtacagtaccattcttgtctaaggtgactgatatcccaatggctcaagttgctacaaacttgattcttggtaaatcattggctgagcaaggttacaaagatggtctttatccagaaagtaaccatgtccatgtcaaagcaccagtcttttcattcacaaaattggctcaggtagatagtctccttggacctgaaatgaaatcaactggtgaagttatgggtacagatgtgactcttgaaaaagcgctctataaagcctttgaagcatcttacctccacttgccaacctttggtaacgtcatctttactattcatgacgataccaaagaagaagcccttgacttggctcgtcgtttcgatgctatcggttatggtatctacgcaactgaaggaacagctaagttcttgaatgaacacggggttcacgcaacgcttgttaacaagttgggtgaaaacgatgacaatgacattccagccctcgttcgtacaggtaaagcacaagctattatcaatacagttggtaacaaacgtacttatgacgaagatggagcagctatccgtagttcagctattgaagcaggaattccactcttcacagcccttgatacagcagatgctatggtgcgtgtgcttgaaagccgcagctttacaacagaagctatctaa

SEQ ID NO: 56 - protein, 1059 aa, synthetic construct
MPKRSDIKKIMVIGSGPIIIGQAAEFDYAGTQACLALKEEGYSVVLVNSNPATIMTDKEIADKVYIEPITLEFVTRILRKERPDALLPTLGGQTGLNMAMELSKAGILDELGVELLGTKLSAIDQAEDRDLFKQLMEELEQPIPESEIVNTVEEAVAFATEIGYPVIVRPAFTLGGTGGGMCANEEELREIAENGLKLSPVTQCLIERSIAGFKEIEYEVMRDAEDNALVVCNMENFDPVGIHTGDSIVFAPTQTLSDIENQMLRDASLKIIRALKIEGGCNVQLALDPHSFKYYVIEVNPRVSRSSALASKATGYPIAKLAAKIAVGLTLDEMINPVTGTTYAMFEPALDYVVAKIPRFPFDKFEHGERRLGTQMKATGEVMAIGRNIEESLLKACRSLEIGVYHNEMSELAEVTDDALVEKVVKAQDDRLFYISEAIRRGYTIEELSELTKIDIFFLDKLLHIFELEQELAAHVGDVDVLKEAKRNGFSDRKIADLWNQTANQVRATRLENNIVPVYKMVDTCAAEFESSTPYFYSTYEWENESIKSDKESVIVLGSGPIRIGQGVEFDYATVHSVKAIQAAGYEAIIMNSNPETVSTDFSVSDKLYFEPLTFEDVMNVIELEQPKGVVVQFGGQTAINLAEPLSKAGVKILGTQVADLDRAEDRDLFEQALKDLDIPQPPGQTATNEEEAVEAARKIGFPVLVRPSYVLGGRAMEIVENEDDLRSYMRTAVKASPDHPVLVDSYIIGRECEVDAISDGKDVLIPGIMEHIERAGVHSGDSMAVYPPQTLSKKIQETIADYTKRLAIGLNCIGMMNIQFVIKDETVYVIEVNPRASRTVPFLSKVTDIPMAQVATNLILGKSLAEQGYKDGLYPESNHVHVKAPVFSFTKLAQVDSLLGPEMKSTGEVMGTDVTLEKALYKAFEASYLHLPTFGNVIFTIHDDTKEEALDLARRFDAIGYGIYATEGTAKFLNEHGVHATLVNKLGENDDNDIPALVRTGKAQAIINTVGNKRTYDEDGAAIRSSAIEAGIPLFTALDTADAMVRVLESRSFTTEAI

SEQ ID NO: 57 - DNA, 3111 bp, synthetic construct
  source 1..3111: mol_type = other DNA
atgtttgcacaattggatacaaaatcagtatatagcttcatggacagtgttgtagatttaaacacctatgtaaaacgtgccaaagagcttggttatcaaagcattggtttgatggatcgtgataatctctatgcggcctatcattttgcccagcttgcaatcagaaatggtttacgtcctttaattggaacagaagctaatttattctatgaagggagaaaaattccctttcgcctattagctaaaaataatcagggctataaaaacttgatgaagttggcaacagagctgtcttcaggacagcgggagtttacatatttttcagactatctgaatgacattgccctaattcttccaagtgaggactgggagcctgggatgagtcttggctcagaagttttcattggggtaagacctgaggatgctggtaaagagtttgattatcctgtggttcctttacatacagtgcgttactttgaaaatgcagatcgctcgactatccagacgttacatgctatctcacaaaatgtgtccttgtctgaggtatccatctgtcctatgaatcagctccttttctcggccaaagaaatggaagaagcttatagtaagcttccagaggctttaaacaatttgaatcagcttgtttcagacgtctcttatcaatttgatacaaatctcaagcttcctcgttttaatcgtgaaatgccagcagttgatcaattgcgtcagttagctcaatctggattagatagtaaagcgttaagagaaccagcttatcaggaacgtttagacaaggaattgtcaataatacatcaaatgggttttgatgactatttcttgattgtttgggatttgttacgttttggtcgtagtcgaggttattatatgggaatgggacgtgggtctgctgcagggagtttggtagcttatgtccttgatattactggaattgatccagttaaaaatgatttacttttcgagcgtttcttaaatgaggaacgttatagtatgcctgatatcgatatagacttacctgatatttatcgaagtgagtttcttcattacgttagagaccgttatggtagtaagcactctgcacagattgttactttttcaacttttggggctaagcaggctatccgagatgtttttaaacgttttggagctaaggaatttgaattatcccaactcagtaaaaaaatatcttttcgagatactttgacaactgtctatgagaaaaatatgtcattcagacagctaattaatcagaggcaggaatttcagagagcttttgaaattgctaaggctattgaaggccagccaagacaaacctccatacatgctgcaggtatcgtgatgagcgatgatgacctgaccaatcatattcctcttaaggcaggtgaggacatgatggttacacagtacgatgcttccgcagtcgaggccaatggtttgcttaagatggatttcttggggcttcgaaatttgacctttgttcaaaaaatgaaggagaagcttgaagaagaacagggaatcgctatcgatatcaaatccattgacttggaagattcacaaaccctagcgctttttgccagtggtaagacaaagggaatttttcaattcgagcaggcaggtgctataaatctccttaaacggattaaacctgctaagttcgaagaagtagtagcgacaacctctctcaaccgtccaggagctagcgattatacagataatttcgttgctcgtaaatatggtaaggagaaagtagatctcattgacccagtggttgctccaattcttgaacctacctatggtatcatgctttatcaggagcaggttatgcagattgcccaagtatttgctggctttagccttggaaaggccgatttgcttcgtcgggcaatgtctaaaaaagatgccaatgaaatgtccaaaatgtccgaacaattcatggaaggaagtcgtcagttagggcgtgacccacaggttgctaaacgtctctttagtatgataactaagtttgcaggatatggttttaataaaagtcatgcctatgcttattcatccctagccttccaattagcttattttaaaacgcattatcctcagatattctatgacgtcatgttaaattattcgtcaagtgattacattagggatgcaattgaaaatggcttttccttagctagcctggatattaatagaattccttatatggacaagatttccgatggaaagattgtactaggtctaaaaaccataaaaggtttgcccagagattttgcactttggattattgaggatcgccaacaaaatggtaaatacatttctatagaggattttttgacacgtattcctagtaaatatcaaaaagtagatattttgacacctttgatccaaataggattatttgatcgttttgagcctaatagacggaaaatcatcggaaacttaagcaacctctttacttttgttaatgaattagggagtttgtttgcagagtcgtcttattcttgggtggagtatgaagactatagtcagacagaacgctacagtatagagcaggatttactaggagttggacttagtacacatccattacatttagctcaaaaaatggcatcacggccctaccaaccaatttctgagttgactttaaacagtaaggctacagttttggtccagcttgaatcagttcgcattatcagaacgaaaaagggagaccagatggctttcctaagtgtttttgatggggtgaataaactggatgtcactctttttccagaaacttatttctatcacaaagataagttaagcgaaggtagtctcttttatctagaaggacgaactcaagagcgcgacggacgtcttcaattaattttgtcaaatatagaggaggctagcactgagcgtttctggattttactagagaatcataataaagatttagaagtttctcacattttatccaagtatcctggacatattcctgtggttattcgttatcagggaagcaaggaaacaatagttagtcaacgatataaagtgtctaagaacgaagaactcagtagggaattagcaccttttgtgttgaaaacggttttacgttaa

SEQ ID NO: 58 - protein, 1036 aa, synthetic construct
MFAQLDTKSVYSFMDSVVDLNTYVKRAKELGYQSIGLMDRDNLYAAYHFAQLAIRNGLRPLIGTEANLFYEGRKIPFRLLAKNNQGYKNLMKLATELSSGQREFTYFSDYLNDIALILPSEDWEPGMSLGSEVFIGVRPEDAGKEFDYPVVPLHTVRYFENADRSTIQTLHAISQNVSLSEVSICPMNQLLFSAKEMEEAYSKLPEALNNLNQLVSDVSYQFDTNLKLPRFNREMPAVDQLRQLAQSGLDSKALREPAYQERLDKELSIIHQMGFDDYFLIVWDLLRFGRSRGYYMGMGRGSAAGSLVAYVLDITGIDPVKNDLLFERFLNEERYSMPDIDIDLPDIYRSEFLHYVRDRYGSKHSAQIVTFSTFGAKQAIRDVFKRFGAKEFELSQLSKKISFRDTLTTVYEKNMSFRQLINQRQEFQRAFEIAKAIEGQPRQTSIHAAGIVMSDDDLTNHIPLKAGEDMMVTQYDASAVEANGLLKMDFLGLRNLTFVQKMKEKLEEEQGIAIDIKSIDLEDSQTLALFASGKTKGIFQFEQAGAINLLKRIKPAKFEEVVATTSLNRPGASDYTDNFVARKYGKEKVDLIDPVVAPILEPTYGIMLYQEQVMQIAQVFAGFSLGKADLLRRAMSKKDANEMSKMSEQFMEGSRQLGRDPQVAKRLFSMITKFAGYGFNKSHAYAYSSLAFQLAYFKTHYPQIFYDVMLNYSSSDYIRDAIENGFSLASLDINRIPYMDKISDGKIVLGLKTIKGLPRDFALWIIEDRQQNGKYISIEDFLTRIPSKYQKVDILTPLIQIGLFDRFEPNRRKIIGNLSNLFTFVNELGSLFAESSYSWVEYEDYSQTERYSIEQDLLGVGLSTHPLHLAQKMASRPYQPISELTLNSKATVLVQLESVRIIRTKKGDQMAFLSVFDGVNKLDVTLFPETYFYHKDKLSEGSLFYLEGRTQERDGRLQLILSNIEEASTERFWILLENHNKDLEVSHILSKYPGHIPVVIRYQGSKETIVSQRYKVSKNEELSRELAPFVLKTVLR

SEQ ID NO: 59 - DNA, 1500 bp, synthetic construct
  source 1..1500: mol_type = other DNA
atgaacatacgagcaagcggtgttttaatgcacatcacatcacttccaggtgactacggaatcggatcttttggtcaagcagcctacgactttgttgatttcttgaaagaaacaaagcaaacatactggcaaattttgccattgactactacaagttatggagattcaccttaccaatcattctcagcaatagcgggaaatactcactttatcgatttggatttccttattcgtgacggattcctgactgaagcagatgttgaaggttctgattttggttcagatccagagtttatcgactacgctaaagtttacgaagcacgacgcccacttcttgaaaaagcagtcaaagcaatcttggcagatgaaaacgaagctaagaagtttgaaactttcaaagctgaaaatgctaactggttggcagattatgctgacttcatggctatcaaagaatactttggtaacaaagcccttcaagattgggatgataaaaaagcgattaaacgtgatgaagcaactcttgaaaagcttcgcaaagaacttactggtgttatcacataccatcaagttgtgcaatacctcttctttagccaatgggcagagttgaaagcctatgctaataagaacggtattcaaattattggtgatatgccaatctacgtttcagctgatagtgtggaagtctggactcaaccacacctcttcaaattagatgctgaatgtaagccacgttacattgccggagttccaccagataacttctcaccaacaggacaactttgggggaatccaatttatgattgggataaacacaaagcagaaaactatgcttggtgggtctcccgtattcaaaaatcattcaaactttttgattgcttgcgtatcgaccacttcaaaggtttctctgatttctgggaaattcctgctggagatgaaactgctgaaaatggtaaatgggttccaggacctggttatgacctcttcaaagtggttaaagaagagttgggtgatcttaagattattgccgaagaccttggtaatattgatgataagacacgtaaacttcttgcagactgtggttacccaggaatgaagattcttcactttggtttctatgataccactggtaacagcatttatgttccacacgcatacacacaacactcagtggcttacactggtacacatgataatgaagttactaatggttggtataataacttgacacaagagcaacgtgattacactgatacttatatcaaccgtcgtcaaggtgaaccaatcacacgtgcccttcttcgcacactcttcgcaacagtaagtaacacagctattgcgactatgcaagatattcttgataaaaatgaaaatagccgtaaaaacatccctaacacagttggaggaaactggagatggcgtatgcttccaggtgaaatcaccgttgataagaaagaatttctgactgatattacagaaagatataatcgaggaaataaataa

SEQ ID NO: 60 - protein, 499 aa, synthetic construct
MNIRASGVLMHITSLPGDYGIGSFGQAAYDFVDFLKETKQTYWQILPLTTTSYGDSPYQSFSAIAGNTHFIDLDFLIRDGFLTEADVEGSDFGSDPEFIDYAKVYEARRPLLEKAVKAILADENEAKKFETFKAENANWLADYADFMAIKEYFGNKALQDWDDKKAIKRDEATLEKLRKELTGVITYHQVVQYLFFSQWAELKAYANKNGIQIIGDMPIYVSADSVEVWTQPHLFKLDAECKPRYIAGVPPDNFSPTGQLWGNPIYDWDKHKAENYAWWVSRIQKSFKLFDCLRIDHFKGFSDFWEIPAGDETAENGKWVPGPGYDLFKVVKEELGDLKIIAEDLGNIDDKTRKLLADCGYPGMKILHFGFYDTTGNSIYVPHAYTQHSVAYTGTHDNEVTNGWYNNLTQEQRDYTDTYINRRQGEPITRALLRTLFATVSNTAIATMQDILDKNENSRKNIPNTVGGNWRWRMLPGEITVDKKEFLTDITERYNRGNK

SEQ ID NO: 61 - DNA, 342 bp, synthetic construct
  source 1..342: mol_type = other DNA
atgaaaaaaatcgaagcgattattcgttcggataaattggaagatttgaaaaataccttgactggtagtggtttgactaagggaatgactgtgagtcaggtcttgggtcatgggacccaacgtggctttgcggagtttgtccgtggtcaacggattgttccgaccttattggctaaggttaaggtggaaatagtagtcaaggatctggcagttgatgaggttgtggatactatccgtagcgctgtggcgacaggcgaagtcggtgatgggaagatttttattgttcctattgatgatgtcattcgtattcgtactggcgaacggggcggagacgctatttaa

SEQ ID NO: 62 - protein, 113 aa, synthetic construct
MKKIEAIIRSDKLEDLKNTLTGSGLTKGMTVSQVLGHGTQRGFAEFVRGQRIVPTLLAKVKVEIVVKDLAVDEVVDTIRSAVATGEVGDGKIFIVPIDDVIRIRTGERGGDAI

SEQ ID NO: 63 - DNA, 1353 bp, synthetic construct
  source 1..1353: mol_type = other DNA
atgacaacaggtaaagaatacgttgctagcgtttttgaaaaagtgaaagctcaaaacagccacgaggctgagttccttcaagcagttgaagaagtcttcaattcacttgtccctgtcttcgataaatatcctaaatatatcgaagaaaaccttttggaacgtttggttgaaccagaacgtatcgtttcattccgtgtaccatgggttgatgataaaggtcaagtacaggtaaaccgtggtttccgtgtacaattctcatcagctattggtccttataaaggtggtcttcgtttccacccatcagtaaaccaatcaatcattaaattccttggtttcgaacaaatctttaaaaactcattgactggtcaaccaatcggtggtggtaaaggtggatcaaacttcgatcctaaaggtaaatcagacaacgaaatcatgcgtttttgccaaagcttcatgactgagttgagcaaacacatcggtgcagatacagacgtgcctgctggtgatatcggtgttggtggacgcgaaattggttacctctatggtcaatacaaacgtattcgtaacgaatacacaggtgttcttactggtaaaggtttgaactggggtggatcacttgctcgtacagaagcaactggttacggtgctgtttactttgcagaacaaatgcttaatgcacgtggtgaaaactttaacggtaaaacagcagttgtttcaggtgcaggtaacgttgcgatttacgctattgaaaaattgcaaagtctaggtgctaaagctgtgacatgttctgattcatctggttttgtttatgatccagatggaatcgacgttgatttgttgaaagaaatcaaagaagttaaacgtgaacgtatcgtgaaatatgcagaagctcgtccaaatgcaactttcacacctgctggtggtgaaaagacagtttggtctatcaaagctgacttggctttcccatgtgcaacacaaaacgaattggatgaaacagatgcagaaacacttgtagcaaatggcgttcttgctgtttcagaaggtgctaacatgccttcaactttgggagctattgatgtcttccttaaagctggtgtatcattcggtcctgccaaagctgctaacgctggtggtgttgctgtatcagctcttgaaatggcacaaaatagcggacgtacatcatggacattcgatgaagttgatgctaaactctacgatatcatgaagggtatctatgaaaatgctgcagctgctgctaaagagtttggtcaagaaggtaaccttgttgtgggtgcaaacattgctggattccttaaagttgcagatgcaatgtcagctcaaggtattgtttaa

SEQ ID NO: 64 - protein, 450 aa, synthetic construct
MTTGKEYVASVFEKVKAQNSHEAEFLQAVEEVFNSLVPVFDKYPKYIEENLLERLVEPERIVSFRVPWVDDKGQVQVNRGFRVQFSSAIGPYKGGLRFHPSVNQSIIKFLGFEQIFKNSLTGQPIGGGKGGSNFDPKGKSDNEIMRFCQSFMTELSKHIGADTDVPAGDIGVGGREIGYLYGQYKRIRNEYTGVLTGKGLNWGGSLARTEATGYGAVYFAEQMLNARGENFNGKTAVVSGAGNVAIYAIEKLQSLGAKAVTCSDSSGFVYDPDGIDVDLLKEIKEVKRERIVKYAEARPNATFTPAGGEKTVWSIKADLAFPCATQNELDETDAETLVANGVLAVSEGANMPSTLGAIDVFLKAGVSFGPAKAANAGGVAVSALEMAQNSGRTSWTFDEVDAKLYDIMKGIYENAAAAAKEFGQEGNLVVGANIAGFLKVADAMSAQGIV

SEQ ID NO: 65 - DNA, 1845 bp, synthetic construct
  source 1..1845: mol_type = other DNA
atgttacaacgtctgaaaaactttttaataggtgaacccttgacccatgataatagcagtgatgatcacttattatctaaggttcaggcattagctatgttgtcgagtgatgctctatcatctatcgcatacggccctgagcaagtcgcattggtcttgatggctgtatcaagcgctgctatttggtggtctatcccaattgggattttggttttagtccttttgattagccttaccatttcttacagacaggtaattaaggcctatccacaaggtggtggagcctacatggtaacttcggaaaatctgtcacctaaatttggtttgatagctggaggtagcctcctagtcgactatatgttgacagttgccgtttcagttgccgcaggtgcagatgccattacctcagccattccattacttcacccctataatcttcaaatctcaataattttggtgcttgttttaatggtgttgaacctcaggggaatgcgtgagtcggcaaagtctttgatggttcctgtttatctctttattgcctcaacgttgttcctattaggatatggtttcttacagatcttaacggggcatatgccttatgcggctacggcgcatttaggccaaccgattacaggtgtgtcagtgattcttattttaagggcctttacttcaggttcggcttcattgacaggggtggaagctatctctaatgcagtgcctttctttaagaaacctaagtctaaaaatgcggcatcaaccctctttatcatgtcaagtattttgggaattatgtttgctggaattacctttcttaactggtggactggtattacacctcattcaggtgtgacaatgttggcgcaactggctagggaaattcttggtgattcatggtttggcagtatcttattttatatttttcagttctcaactgccatgattcttgcagtggcagcaaatactggtttttcagcctttccgatgttgtcctttaacatggctaagaataaatacatgcctcatatctatcttgaaaaaggtgcacgtatggcctactctaacggtatcttgacgctggctattggggccattactctcttattcattttccgaggaaatacggaacgtttgattccgctttatacaataggggtgtttgttccttttgcactttctcaaacaggtatggttgttcattggagtcgtgaatatggcaaggatttctggaagcattctttagctaatattttaggtgccttaatttgttacggtattgttctcattctcattctcttccgtttaggggatatttggccattcttcccaattattgctattttgatgtggatgtttcttaagattaagaaacattacgatggggttgctcaacagttgcgtattgatggcccaatcaaagaacatcattatcaaggtaacattgtccttatcttggtgggaaatgttacaaaggctaatattggtgccatttcttatgcacgttctattggtgataaggttattgctatgcacgtgtctactaaagatacagagaataaggataaggaaatcgaacaagagtttaaaaaatatttcccagaaatcgagatggttcatatccgatcaccttacagctcaattacacagtcaactattcaatatgtagacgaagtggctactcaggctgataaggataatgcgactctgactgttttgattccacagtttgttccgaaaaaatcatggcaaaatatacttcacaaccagacgagtttacgattgaaatattatctcaaatggcgtgaaaatattgtcatttcatcatattcttaccacttaaaagattaa

SEQ ID NO: 66 - protein, 614 aa, synthetic construct
MLQRLKNFLIGEPLTHDNSSDDHLLSKVQALAMLSSDALSSIAYGPEQVALVLMAVSSAAIWWSIPIGILVLVLLISLTISYRQVIKAYPQGGGAYMVTSENLSPKFGLIAGGSLLVDYMLTVAVSVAAGADAITSAIPLLHPYNLQISIILVLVLMVLNLRGMRESAKSLMVPVYLFIASTLFLLGYGFLQILTGHMPYAATAHLGQPITGVSVILILRAFTSGSASLTGVEAISNAVPFFKKPKSKNAASTLFIMSSILGIMFAGITFLNWWTGITPHSGVTMLAQLAREILGDSWFGSILFYIFQFSTAMILAVAANTGFSAFPMLSFNMAKNKYMPHIYLEKGARMAYSNGILTLAIGAITLLFIFRGNTERLIPLYTIGVFVPFALSQTGMVVHWSREYGKDFWKHSLANILGALICYGIVLILILFRLGDIWPFFPIIAILMWMFLKIKKHYDGVAQQLRIDGPIKEHHYQGNIVLILVGNVTKANIGAISYARSIGDKVIAMHVSTKDTENKDKEIEQEFKKYFPEIEMVHIRSPYSSITQSTIQYVDEVATQADKDNATLTVLIPQFVPKKSWQNILHNQTSLRLKYYLKWRENIVISSYSYHLKD

SEQ ID NO: 67 - DNA, 3507 bp, synthetic construct
  source 1..3507: mol_type = other DNA
atggctaatacaatcttagagctttttgagaagaatcatcaattgcttgagtggcgtgataaggtaagcttattgagtaggcaattggtcatgggattttctggttcgagcaaggcagttgccatggcatctgccctttctaatcaggttcctaagatttttatagttacctcaacacaaaatgaagctgagcaattagtaggagatttgtcagctatcttaggagaggataaggtttattctttttttgcggatgatgtatcagctgctgaatttatctttgcttcaccagaaaaaactcattctcgtcttgaaagtcttaactttttaatggatgaagaggcaagtggggtcttggttacaagtttagttggtacgaagttacatttgcctaatcctagggtttataaggactctcgaatcgacttagtactagggaaggaacatgatttagatgctctcagtaggcatctaacgcatattgggtatcaaagagtagagcaagttctttcacctggtgagtttagtcgtaggggggatattttagatatttacgaattgactgcagagcttccctaccgattagagttttttggagatgagattgatggcattcgtcagtttgatagcgatagtcagaaatctttaaacaatttagaacatgttgtcatatcaccggcggatgatattatcttaacaagagaagattaccaacgtgctgagaaggccctagaaagtgcgatctcgagggcagacggtcttcataaggcttaccttgaggaagttctatcagtgacgattgatggtcatcgccataaagatttacgtaagtttttgtctctcttttatgacaaagcttataccttatttgactatcttccaaaagggactcctgtattcattgatgattttcaaaagatagttgaccgccatggacgtcttgaattggaagtggctaatcttttaactgaggatcttcatcaaggtaagtctctgagtcatctaaactacttcgtggattcatttaagactttacgtaactatcagccagctagcttcttttctaatttccataatggattaggaaatcttaaatttgataagttgtatcaattcacacaataccctatgcaggagtttttcaaccaatttccattactgattgacgaaattcgtcgttatactaagaataaggcaacggttattttacaagtagggtctgataagcaacttaattctttgaaggaaaccttggaagagtacaatctagatttacctcttagtagctttggagatttgatacctcataaggctaaactagtcctaggaaacttatcgaatggcttttactttgcagatgaaaaggttgttctggttactgaacgtgagatttttcataaaaaagtaaaacgtcgtacgcgtgctaaccatatttctaatgctgagcgtcttaaagactataatgaacttgaaaaaggtgattatgttgtacaccagactcatggtataggacaatttaaaggtattgagactattgaaattaaaggggtacaccgtgactacttgaccattcaatatcaggatgctgcaacaatttctctacctattgagcagatcgagagtctctctaagtatgtgtcagtggacgggaaagagcctaaaattaataaactcaatgatgggcgtttccaaaagaccaaacaaaaagtaagcaagcaggtagaggatattgctgataatctcttaaaactctatgccgagcgaagtcaactgaaaggatttgctttttcaccggatgatgataatcaaagggattttgaagatgagttctcctatgttgagacagtggatcaactgcgttccattaaggaaattaaggcggacatggagtcagacaaacctatggatcgtctcttggttggtgatgttggttttggtaagacagaggtagccatgcgtgctgccttcaaggcggttaatgatgggaagcaagttgctatcctagttcctaccacagttcttgcccatcagcattatatgaattttaaagagcgttttgaaaatcatgctgttgaagtagatgaactcagtcgttttagaagtaaaaaagagcaaaatgagactattaaaaatctagctaaaggacgtattgatattattattggtacccaccgcttgttgtctaaagatgttaagttttctgatttaggcttgctagtaattgatgaagaacaacgttttggcgtgaagcataaagagaaacttaaggagttgaaagctaaagttgatgtccttactctaactgcgacccctattcctcgtacacttcatatgtctatgttgggtattcgtgatttgtcgattattgagacagcacctaccaaccgttatccagttcaaacttatgttatggaaaccaatcctggtcttattcgagaagcgatattaagggagatggacagaggtggccagattttttatatttataatcgtgttgaaaccattgatcaaaaagtttctgaactgcatgagctagtccctgaagcacgtatcggatttgttcatggtcagatgagtgaagttatgttggagaatactctcttagattttttgaatggtgattacgatattttggtagcaacgaccattatcgagacaggcattgatatccccaatgttaatacacttctcattgagaatgcagaccatatgggcttgtctaccctttatcagctaagaggtcgtgtgggacgtagtaatcgaattgcctatgcctaccttatgtatcatcctgacaaagtgttgacggaagtctcagagaagcgtttggatgctattaagggatttacagaactgggatcaggtttcaaaattgctatgcgtgatttatctattcgcggtgcagggaatattcttggtgcttctcaaagtggtttcatcgactcagtcggatttgaaatgtactcgcaactcttggaagaggctatcaataagcgtcaaggtaagactcaggttcgtcgaaaaggaaatgcagagtttaatcttcagattgatgcttaccttccttctgactatatttctgacgagcgccaaaaaattgaaatttataagcgtattcgtgaaattgagaatcagaaagactaccaggaccttcaagatgagttgattgaccgctttggagaatatccagatcaagttgcctatctcattgagattggtctggttaaggcctatttagatgctgcctttgcagaattagtcgagcgtaagaacgatactatcatcgttcgtttcgaaaaagtctctctaaaattcttcttgacacaagactattttgaagccatctctaagacgaatttaaaggcaagaattagtgataaccaaggaaaagtagagattatttttgatgttcgtaacaagaaagactatgaaatccttgaggaattaaagatttttggtcagacatttatggaaattaaaaaacgtaaagaaaaaaaagaaacttaa

SEQ ID NO: 68 - protein, 1168 aa, synthetic construct
MANTILELFEKNHQLLEWRDKVSLLSRQLVMGFSGSSKAVAMASALSNQVPKIFIVTSTQNEAEQLVGDLSAILGEDKVYSFFADDVSAAEFIFASPEKTHSRLESLNFLMDEEASGVLVTSLVGTKLHLPNPRVYKDSRIDLVLGKEHDLDALSRHLTHIGYQRVEQVLSPGEFSRRGDILDIYELTAELPYRLEFFGDEIDGIRQFDSDSQKSLNNLEHVVISPADDIILTREDYQRAEKALESAISRADGLHKAYLEEVLSVTIDGHRHKDLRKFLSLFYDKAYTLFDYLPKGTPVFIDDFQKIVDRHGRLELEVANLLTEDLHQGKSLSHLNYFVDSFKTLRNYQPASFFSNFHNGLGNLKFDKLYQFTQYPMQEFFNQFPLLIDEIRRYTKNKATVILQVGSDKQLNSLKETLEEYNLDLPLSSFGDLIPHKAKLVLGNLSNGFYFADEKVVLVTEREIFHKKVKRRTRANHISNAERLKDYNELEKGDYVVHQTHGIGQFKGIETIEIKGVHRDYLTIQYQDAATISLPIEQIESLSKYVSVDGKEPKINKLNDGRFQKTKQKVSKQVEDIADNLLKLYAERSQLKGFAFSPDDDNQRDFEDEFSYVETVDQLRSIKEIKADMESDKPMDRLLVGDVGFGKTEVAMRAAFKAVNDGKQVAILVPTTVLAHQHYMNFKERFENHAVEVDELSRFRSKKEQNETIKNLAKGRIDIIIGTHRLLSKDVKFSDLGLLVIDEEQRFGVKHKEKLKELKAKVDVLTLTATPIPRTLHMSMLGIRDLSIIETAPTNRYPVQTYVMETNPGLIREAILREMDRGGQIFYIYNRVETIDQKVSELHELVPEARIGFVHGQMSEVMLENTLLDFLNGDYDILVATTIIETGIDIPNVNTLLIENADHMGLSTLYQLRGRVGRSNRIAYAYLMYHPDKVLTEVSEKRLDAIKGFTELGSGFKIAMRDLSIRGAGNILGASQSGFIDSVGFEMYSQLLEEAINKRQGKTQVRRKGNAEFNLQIDAYLPSDYISDERQKIEIYKRIREIENQKDYQDLQDELIDRFGEYPDQVAYLIEIGLVKAYLDAAFAELVERKNDTIIVRFEKVSLKFFLTQDYFEAISKTNLKARISDNQGKVEIIFDVRNKKDYEILEELKIFGQTFMEIKKRKEKKET

SEQ ID NO: 69 - DNA, 1104 bp, synthetic construct
  source 1..1104: mol_type = other DNA
atgctagagttccctctaattaatgatacgagtcggaaaattattcatatcgacatggatgccttttttgctcaggttgagatgagggatgaccctagtcttaaagataaacctgttattatcggtaatgaccccagaaaaacaggcggtcgtggtgtggtgtctacttgtaactacgaggctagaaaatacggtgtccactcagccatgtcttctaaggaagcctacgaacgttgtcccaatgctgtctttatttcaggaaattatagccattaccgagaggtagggatgcagattcgtgaaatttttaaatgctacacggacttggttgagcctatgtctattgacgaggcctatctcgatgttacgaccaataaacttgggatcaagtcggccgttaaggtcgctaaacttatccagtatgatatctggcaggaactccatctgacctgttcggctggtgtttcctacaataaattcattgcgaagctggcttctgatttccaaaaacctgctggtttgacggtagtacttcccgaagaggcccaggaatttttgaaaaagctacctattgcaaagtttcatggtgttggtaaaaagtcagttgagcgtcttcatgacatggatatatacaccggtgccgatcttttgaaaatttctgagataaccttaatagaccgctttggacgttttggctttgacctctttcgcaaggctagaggcatttccaatagcccagtaaagcctaatcgtgtccgtaaatctatcggaagtgagcgaacctatggtaagctcctgtataatgaagatgacatcaaggctgagttgactaaaaatgctaaacgggtagcggaaagcgcccagaaagctaaaaaagtcggtcgtatcattgtcatcaaggttcgttattcggacttctcaaccctaaccaaacgtatgaccttggacaaatcaacccaggattttgacaccatcgataggatttctcatagtatttttgatcagctggaggaaaatagtagtggcgttcgcctgcttggtgttacgctgacaggtttggaagaccaagaaggtagacagttagacttagacgatttggcgtga

SEQ ID NO: 70 - protein, 367 aa, synthetic construct
MLEFPLINDTSRKIIHIDMDAFFAQVEMRDDPSLKDKPVIIGNDPRKTGGRGVVSTCNYEARKYGVHSAMSSKEAYERCPNAVFISGNYSHYREVGMQIREIFKCYTDLVEPMSIDEAYLDVTTNKLGIKSAVKVAKLIQYDIWQELHLTCSAGVSYNKFIAKLASDFQKPAGLTVVLPEEAQEFLKKLPIAKFHGVGKKSVERLHDMDIYTGADLLKISEITLIDRFGRFGFDLFRKARGISNSPVKPNRVRKSIGSERTYGKLLYNEDDIKAELTKNAKRVAESAQKAKKVGRIIVIKVRYSDFSTLTKRMTLDKSTQDFDTIDRISHSIFDQLEENSSGVRLLGVTLTGLEDQEGRQLDLDDLA

SEQ ID NO: 71 - DNA, 1104 bp, synthetic construct; identical to SEQ ID NO: 69
  source 1..1104: mol_type = other DNA

SEQ ID NO: 72 - protein, 367 aa, synthetic construct; identical to SEQ ID NO: 70

SEQ ID NO: 73 - DNA, 1872 bp, synthetic construct
  source 1..1872: mol_type = other DNA
atgatccaagttggcaaattgtttgctggacgttatcgaatccttaaatctatcggacgtggtggtatggcggatgtttatctggctaacgacctgattctggataatgaaagtgtagctattaaggtactacgtaccaattatcaaactgatcaggttgctgtggcgcgtttccagcgtgaagcacgtgccatgactgagcttagccatccgaatatcgttgcgattagagatattggtgaagaagacggtcagcagttcttggtgatggagtatgtggatggttcggacttgaagaaattcattcaagaccatgcaccattgtctaaccaagatgttgtgcgtattatgggggaagtcctttccgccatgactcttgcccaccaaaagggtattattcaccgtgaccttaagccacagaacgttctattaactaaggatggtagagctaaggttacagactttggtatcgcagtagcatttgctgaaaccagcctgacacaaactaactcaatgttggggtcagtccattatttgtcaccagaacaggcacgtggttctaaagccactatccaaagtgatatctatgctatggggattatgctctttgaaatgttgacagggcatatcccttttgatggcgatagtgcagtaaccattgcacttcaacatttccaaaagcccctgccatcgattttggctgagaaccgtaacgttcctcaagctttagaaaatgttgttattagagcaactgctaaaaagcttgaaaatcgttacaacagcactttggaaatgagtcgcgacctagcaactagtttgcatcctagtcatagtcgtgattctaaggtagtttttgatgacatgacggatactaagaccctgccaaaggtcgattcagttccatcagtaggtcctgagaaaaagatttctcctaaaccatcagagcctacaccggctccaagcaaacaaccgagggaaaaagcttctctatctaaaaaaaagaaaaatctctttttaactctactcaaggttttactaggcttcgtctttataggaattatcatttttgcctatcttgtttttaccaatcctgacaatgtccaagtaccaaatgttgtaggtcaagagttgtcaacagctcagacgaaacttgaaagtgcaggctttaaggttggcgatgttaaagaagtcgaaaatgattcagtcaataagggcaaagttatcaagaccgaccctacagctgggacaatgcgtaaagagggagcaagcattgatgtctatgtttcttctggtagcaaaggtttcactctgaaaaattacaaggggaaaaattataaggatgccattgaggatttaacctcgaattatggggtttccgaggatcagatcgatattcaacatgtcgaagacgataatgctaaagaaggcgaaattctatctcaaagttcggagaaaaacaagtccttcaatcctaaagatagtaaggcaaaaatcaagtttcgtgtggcaactcctaagactataactatgcctgatgtgactggtttaactgtctcgacagcagtccaaaccttgaatagtaaagacatttcaagcagcagtattgagtatcatgactataatactggtgttaagttggataagagtaaggtacctagtaatacagaggtcctttatcaagatcctcaagttggtactgctgttgatggcacagtgattctctatgtttcagtcgtaactaagagttcaagtattcaacagtcaagctcagcaagtacgacacaatcgtcgtcatctacaagttctagttctgaaagtagtacagcttctagtaccgaaacagctacagaagctactcacacagagcaataa

SEQ ID NO: 74 - protein, 623 aa, synthetic construct
MIQVGKLFAGRYRILKSIGRGGMADVYLANDLILDNESVAIKVLRTNYQTDQVAVARFQREARAMTELSHPNIVAIRDIGEEDGQQFLVMEYVDGSDLKKFIQDHAPLSNQDVVRIMGEVLSAMTLAHQKGIIHRDLKPQNVLLTKDGRAKVTDFGIAVAFAETSLTQTNSMLGSVHYLSPEQARGSKATIQSDIYAMGIMLFEMLTGHIPFDGDSAVTIALQHFQKPLPSILAENRNVPQALENVVIRATAKKLENRYNSTLEMSRDLATSLHPSHSRDSKVVFDDMTDTKTLPKVDSVPSVGPEKKISPKPSEPTPAPSKQPREKASLSKKKKNLFLTLLKVLLGFVFIGIIIFAYLVFTNPDNVQVPNVVGQELSTAQTKLESAGFKVGDVKEVENDSVNKGKVIKTDPTAGTMRKEGASIDVYVSSGSKGFTLKNYKGKNYKDAIEDLTSNYGVSEDQIDIQHVEDDNAKEGEILSQSSEKNKSFNPKDSKAKIKFRVATPKTITMPDVTGLTVSTAVQTLNSKDISSSSIEYHDYNTGVKLDKSKVPSNTEVLYQDPQVGTAVDGTVILYVSVVTKSSSIQQSSSASTTQSSSSTSSSSESSTASSTETATEATHTEQ

SEQ ID NO: 75 - DNA, 981 bp, synthetic construct
  source 1..981: mol_type = other DNA
atgaaaaaaacgaccttagccttaggaatgcacgataaacttttcaaacgtgcccgtaccatgtatcagattgagcattgcatttgtgatttattgatgacaaagggttttcttcgtattgagacaccaaccttagagcattttgaagtcttcagtgatgtggtggacaatggtaactataatttctttgataaaaatggagatcttattagcctacgtcctgatattaccagtcaaatcgggcgcgtaattgcttccacacaagttcatacaccgattaaattttcctactctggaaaggtctttaactacaacgaagagatgcgtggcttgtccaatgagcatacgcaggctggtgtggaaatcattggttttccagttcatcaggcattggaagaagctgtgatatctgctaaagaggccttggatgtggcaggtgtcagaaattacaagtttgaattttcacatgcccagctcctgcaactcatttttgaagaattaaaccttccagcagtaaaagaagcagagttggccacctatatccgtgataaatcaatcacaggtctcaaggagttcactaaagaaaatccaagtcaatatgacaaggtcttggagcagttgccatccctttttggtgagacaactgctgtcttgaccgaggcgagacagttaaccgacaatgaatcctttttgacagctttggatagtttggaagtcttaaccagtcgcctagctgataatctcccagagacgacgctcgatttggctcaattaccagcagtgccttactacacgggcataatgtttaaggtcttcggtgataaggtaccggatgcttttgtatcaggtggccgctacgataaactatttgaacgttttggggctaccgagttaacagcggttggttgggctattgatatcgactcagtttatcaagcagtacatgacgatgtggaatttggaggtgacatggatgactag

SEQ ID NO: 76 - protein, 326 aa, synthetic construct
MKKTTLALGMHDKLFKRARTMYQIEHCICDLLMTKGFLRIETPTLEHFEVFSDVVDNGNYNFFDKNGDLISLRPDITSQIGRVIASTQVHTPIKFSYSGKVFNYNEEMRGLSNEHTQAGVEIIGFPVHQALEEAVISAKEALDVAGVRNYKFEFSHAQLLQLIFEELNLPAVKEAELATYIRDKSITGLKEFTKENPSQYDKVLEQLPSLFGETTAVLTEARQLTDNESFLTALDSLEVLTSRLADNLPETTLDLAQLPAVPYYTGIMFKVFGDKVPDAFVSGGRYDKLFERFGATELTAVGWAIDIDSVYQAVHDDVEFGGDMDD

SEQ ID NO: 77 - DNA, 435 bp, synthetic construct
  source 1..435: mol_type = other DNA
atgggcttttcaagtcttttaagcactccttatttaggacgacttggtgatcgcattgggaatcatcgtttgctcttaatgggcttattctatagtttccttttgtatttcctttgtggttttgcaggttcagccttgcaacttggccttttacgctttgcctatggttttggtgtaggtgccttgacgcctagtgtcaattccttattgaccaagatgaccccaaaagaaggaatttcaagaatctttagccttaatcaaagtttttcatacattggacaagttttaggaccctttgtgggttcagctgttgcaacaggattaggctatcgttgggttttctttgtgacagcagccatcgtctttggaaattttgtttgttccttaatcatctttagaaaatcgctaggagttaagaatataggtgagagttaa

SEQ ID NO: 78 - protein, 144 aa, synthetic construct
MGFSSLLSTPYLGRLGDRIGNHRLLLMGLFYSFLLYFLCGFAGSALQLGLLRFAYGFGVGALTPSVNSLLTKMTPKEGISRIFSLNQSFSYIGQVLGPFVGSAVATGLGYRWVFFVTAAIVFGNFVCSLIIFRKSLGVKNIGES

SEQ ID NO: 79 - DNA, 1824 bp, synthetic construct
  source 1..1824: mol_type = other DNA
atgtctaaaattattggtattgacttaggtacaacaaactcagcagttgcagttcttgaaggaactgaaccaaaaatcatcgctaacccagaaggaaatcgtacaactccatctgtagtttcattcaaaaatggtgaaatcatcgttggtgatgctgcaaaacgtcaagcagtcacaaacccagatacagtaatctctatcaaatctaagatgggtacttctgaaaaagtttctgctaacggcaaagaatacactccacaagaaatctcagcaatgatccttcagtacttgaaaagttatgctgaagaatacctcggtgaaaaagtaaccaaggctgtcatcacagttccagcttactttaacgatgctcaacgtcaagcaactaaagatgctggtaaaatcgctggtcttgaagtagaacgtatcgtcaacgaaccaactgcagcagcgcttgcttatggtttggacaagactgacaaagaagaaaaaatcttggtatttgaccttggtggtggtacattcgacgtatctatccttgaactcggtgatggtgtctttgatgtattggctactgcaggggacaacaaacttggtggtgatgactttgaccaaaaaattatcgattacatggttgaagaattcaagaaagaaaacggcatcgacttgtcaacagacaagatggctcttcaacgtttgaaagacgcagcagaaaaagctaagaaagacctttcaggtgtaacttcaacacaaatcagcttgccgttcatcacggctggtgaagctggacctcttcacttggaaatgactttgactcgtgccaaatttgatgatttgactcgtgaccttgtagaacgtactaaaactccagttcgtcaagccctttcagatgcaggtttaagcttatcagacattgacgaagtcatccttgttggtggttcaactcgtatcccagctgttgtagaagcagttaaggctgaaactggtaaagaaccaaacaaatcagtaaaccctgatgaagtagttgctatgggtgctgctatccaaggtggtgtgatctctggtgatgtgaaagacgttgtccttcttgacgtaacaccattgtcacttggtatcgaaacaatgggtggcgtcttcacaaaactcatcgaacgtaacacaacaattccaacatctaaatcacaagttttctcaactgcagcagataatcaaccagccgttgatattcacgttcttcaaggtgaacgcccaatggcagcagataacaagactcttggacgtttccaattgactgatatcccagctgcacctcgtggtgtcccacaaatcgaagtaacatttgacattgataagaacggtatcgtatctgttaaagcgaaagatcttggaactcaaaaagaacaaacaattgtcatccaatctaactcaggtttgactgatgaagaaattgaaagaatgatgaaagatgctgaagcaaatgctgaagcagatgcaaaacgtaaagcagaagtagaacttcgtaacgaagtcgaccaagctatctttgccactgaaaaaacaattaaagaaacagaaggtaaaggctttgatacagaacgtgatgctgcccaaacagcacttgatgagcttaagaaagctcaagaatcaggtaatcttgatgacatgaaggctaaattggaagctcttaacgaaaaagcccaagctttggcagttaaactctacgaacaagcagcagcggcacaacaagctcaagcaggagcagaaggcgcacaagcaacagataactcaggcgacgatgtcgtagacggagagtttactgaaaagtaa

SEQ ID NO: 80 - protein, 607 aa, synthetic construct
MSKIIGIDLGTTNSAVAVLEGTEPKIIANPEGNRTTPSVVSFKNGEIIVGDAAKRQAVTNPDTVISIKSKMGTSEKVSANGKEYTPQEISAMILQYLKSYAEEYLGEKVTKAVITVPAYFNDAQRQATKDAGKIAGLEVERIVNEPTAAALAYGLDKTDKEEKILVFDLGGGTFDVSILELGDGVFDVLATAGDNKLGGDDFDQKIIDYMVEEFKKENGIDLSTDKMALQRLKDAAEKAKKDLSGVTSTQISLPFITAGEAGPLHLEMTLTRAKFDDLTRDLVERTKTPVRQALSDAGLSLSDIDEVILVGGSTRIPAVVEAVKAETGKEPNKSVNPDEVVAMGAAIQGGVISGDVKDVVLLDVTPLSLGIETMGGVFTKLIERNTTIPTSKSQVFSTAADNQPAVDIHVLQGERPMAADNKTLGRFQLTDIPAAPRGVPQIEVTFDIDKNGIVSVKAKDLGTQKEQTIVIQSNSGLTDEEIERMMKDAEANAEADAKRKAEVELRNEVDQAIFATEKTIKETEGKGFDTERDAAQTALDELKKAQESGNLDDMKAKLEALNEKAQALAVKLYEQAAAAQQAQAGAEGAQATDNSGDDVVDGEFTEK

SEQ ID NO: 81 - DNA, 1155 bp, synthetic construct
  source 1..1155: mol_type = other DNA
ttgactaatccgattattgcctttcaaaacgtctcgaaagtatttgaagacagtggcacacaagttttaaaagacatcaactttgaactggaagaaggtaagttctataccctgcttggggcttctggttctggtaaatcaactatcttgaacatcattgctggtctcttagatgcgaccagtggtgacgttcttttggatggaaagcgtattaatgatatccctatcaataaaagggatgttcacaccgttttccaatcttatgccctctttccacacatgaatgtctttgacaatgtggcttttgcgcttaagttaaaaaaagtgcctaagaaggaaattgaagagcgtgtaaaagaagcattgaaaatggttcaactagatggttatcaaaagcgctcgattcaaaaattatcgggtggtcaacgccaacgtgtagctattgcccgtgcgattatcaatcagccacgtgtggtccttttggatgagcccttgtcagcactggatttgaagttacgtactgaaatgcaatacgagttgcgtgaactacagaaacgtctagggattacttttgtctttgtcacccacgaccaggaagaagccttggccatgtcagactggatttttgttatgaacgatggtgaaattgttcaatcaggaacgcctgttgatatctatgacgagcctatcaaccatttcgttgcaacctttatcggagaatctaatatcctaccaggagtcatgattgaggactatttggtcgagttcaacggaaaacgtttcgaatctgttgatggaggaatgcgtccaaatgaacctgtcgaagtggttatccgtcctgaagatttacaaatcacgcttcctgaggaaggtaagcttcaggttcgtgtagaaatacagcttttccgtggggtccactatgaaatcattgcctatgataatcttggcaatgaatggatgattcattcaacccgtaaggcgattgagggtgagattattggccttgattttacgcctgaagatattcatatcatgcgtcttaatgagacagaggaagaattcgatgctcgtattgaagaatatgtggaaatggaagagcctgaggatggcttgattaatgccattgaggaggagcgtcatgaagaaaacagctag

SEQ ID NO: 82 - protein, 384 aa, synthetic construct
LTNPIIAFQNVSKVFEDSGTQVLKDINFELEEGKFYTLLGASGSGKSTILNIIAGLLDATSGDVLLDGKRINDIPINKRDVHTVFQSYALFPHMNVFDNVAFALKLKKVPKKEIEERVKEALKMVQLDGYQKRSIQKLSGGQRQRVAIARAIINQPRVVLLDEPLSALDLKLRTEMQYELRELQKRLGITFVFVTHDQEEALAMSDWIFVMNDGEIVQSGTPVDIYDEPINHFVATFIGESNILPGVMIEDYLVEFNGKRFESVDGGMRPNEPVEVVIRPEDLQITLPEEGKLQVRVEIQLFRGVHYEIIAYDNLGNEWMIHSTRKAIEGEIIGLDFTPEDIHIMRLNETEEEFDARIEEYVEMEEPEDGLINAIEEERHEENS

SEQ ID NO: 83 - DNA, 1737 bp, synthetic construct
  source 1..1737: mol_type = other DNA
atgcctaagctcggtgttgatatgcaggaaggtgaaatcattgagtggaaaaaacaagagggagacgtggttaatgagggagatatccttcttgaaatcatgtcagataagacaaacatggaacttgaagcagaagactcaggtgttcttttgaagattacccgtcaagctggtgaaacagtaccagttactgaggtgattggttatatcggagctgaaggagaagttgtggctgataattctacaagcgcaccagctgcagaagcaactgctcaattggaagcggctgggcttgaagtccctaaagctccatctcaaccatctccagcgacagcagaaaaagcagcccttgctgataacgaatatgatatcgttgttgttggtggtggtccagctggttactatgcagccatccgtggagcacaacttggcggtaaggttgccatcgttgaaaaatcagaatttggtggaacatgtttgaataagggatgtatcccaactaagacctaccttaaaaacgcagaaatccttgatggacttaaaattgcagcaggtcgtggtatcaacctagcatcaacaaactacaccatcgatatggataagacggttgactttaaaaactctgttgttaagacccttactggtggtgtgcaaggtcttttgaaagctaataaggtaactattttcaatggtcttggtcaagttaaccctgataagactgtcactattggctcagaaacaatcaagggacgtaacatcatccttgcaactggatcaaaagtatcacgtattgatatccctggaatcgattcacaacttgtgatgacatcagatgatattcttgatttgcgtgaattgcctaaatcacttgctgttatgggtggtggtgtcgttggtatcgagcttggtcttgtttatgcttcatacggcgttgacgtaacagttgtagagatggctgatcgtatcatacctgctatggataaagaagtgtcacttgagcttcaaaaaatcctttctaagaagggtatgaagattatgacatctgttggtgtttctgaaattgttgaaggcaataaccaattaacacttaaactcaataatggcgaagagattgtggctgaacgtgcccttctatcaatcgggcgtgttccacaattgaacggtcttgaaaatcttaaccttgagctcgaccgtggccgtatcaaagtaaatgaataccaagaaacttctatccctggtatttacgcccctggtgatgtaaacggaactaagatgcttgctcacgctgcttaccgtatgggtgaagtagcagccgaaaatgctatgcatggtaatgtccgtaaggcacaccttgattatacaccagcggcagtttacacccacccagaggtagcaatgtgtggtctaactgaagaagatgcacgcgctaaatatggtgatgttctcattggtaaatcaagctttgctgggaatggacgtgctatcgcctcaaatgaagctcatggctttgttaaagtcgttgcggatgcgaaataccatgaaatccttggagttcacattattggtccggctgctgctgagttgattaacgaagcagcaactattatggaaaatgaactgactgtagatgaactcttgcaatctatccatggtcacccaactttctctgaaaatatgtatgaggcttttgctgatgtacttggagaagctgttcataacccaccaaaacgtaaataa

SEQ ID NO: 84 - protein, 578 aa, synthetic construct
MPKLGVDMQEGEIIEWKKQEGDVVNEGDILLEIMSDKTNMELEAEDSGVLLKITRQAGETVPVTEVIGYIGAEGEVVADNSTSAPAAEATAQLEAAGLEVPKAPSQPSPATAEKAALADNEYDIVVVGGGPAGYYAAIRGAQLGGKVAIVEKSEFGGTCLNKGCIPTKTYLKNAEILDGLKIAAGRGINLASTNYTIDMDKTVDFKNSVVKTLTGGVQGLLKANKVTIFNGLGQVNPDKTVTIGSETIKGRNIILATGSKVSRIDIPGIDSQLVMTSDDILDLRELPKSLAVMGGGVVGIELGLVYASYGVDVTVVEMADRIIPAMDKEVSLELQKILSKKGMKIMTSVGVSEIVEGNNQLTLKLNNGEEIVAERALLSIGRVPQLNGLENLNLELDRGRIKVNEYQETSIPGIYAPGDVNGTKMLAHAAYRMGEVAAENAMHGNVRKAHLDYTPAAVYTHPEVAMCGLTEEDARAKYGDVLIGKSSFAGNGRAIASNEAHGFVKVVADAKYHEILGVHIIGPAAAELINEAATIMENELTVDELLQSIHGHPTFSENMYEAFADVLGEAVHNPPKRK

SEQ ID NO: 85 - DNA, 2619 bp, synthetic construct
  source 1..2619: mol_type = other DNA
atgaaacaacttacttcagcacaaattcgccaaatgtggcttgatttctggaaatcaaaaggtcatgctgttgaaccatcagctaacttggttccagttaacgacccaactcttctttggattaactcaggggttgccactctgaagaaatactttgatggctctgtgattcctgaaaatccacgtatcaccaactcacaaaaggctatccgtaccaatgatatcgaaaacgttggtaagacggctcgtcaccacacgatgtttgaaatgcttggtaacttctcagttggtgattacttccgtgatgaggctatcgagtggggttatgagcttttgacaagcccagagtggtttgatcttccaaaagacaaactttacatgacttattacccggatgataaagattcatacaaccgttggattgcttgtggagttgagccaagtcacttaattccaatcgaagataatttctgggaaatcggtgcaggtccttctggaccagatactgagatcttctttgatcgtggagaagaatttgaccctgacaatattggtattcgcttgcttgaagaggatattgaaaacgaccgttacattgaaatttggaacatcgttttgtcacaatttaatgcagatccagctgttccacgttcagaatacaaagagcttcctaataaaaacattgatacgggtgcaggtcttgaacgtcttgcagccgtaatgcaaggggctaaaacaaactttgaaacagaccttttcatccctattattcgtgaaattgaaaaaatgtctggtaaagcttatgatccagatggtgaaacattgagcttcaaagttatcgcagaccacatacgttcattggcctttgctatcggtgatggagcccttccaggtaatgaaggccgtggttatgttcttcgtcgtcttctccgtcgtgccgtaatgcacggtcgtcgtcttggtatttctgatgctttcttgtataaacttgtcccaactgttggtcaaatcatggaaagctattacccagaagttcttgagaagaaagactttatcgagaaaattgttaaacgtgaagaagaaacctttgcacgtactatcgatgcaggttcaagcatgcttgatgaacttcttgctaaccttaagaaatctggtaaagatacgcttgaaggtaaagatatctttaaactttacgatacttacggattcccagtggaattgacagaagaattagctgaagacgaaggcttcaagattgaccatgaaggtttcaaagctgccatgaaagaacaacaagaccgtgcgcgtgctagcgtggttaaaggtgggtctatgggtatgcaaaatgaaactcttgctaacatcactgaaccatctgagttcttgtatgaagcggaaactgcagaaagtcgtttgagcgtaatcgttgctgatgatgctcgtcatgatagtgtaaattctggccaggctcttcttgtctttgaacaaacaccattctacgctgaaatgggtggacaagtagcagaccacggtactatttcagatgcagcgggtactactgttgcgcgtgttgttgatgttcaacgtgcaccaaatggacaagctcttcatactgttgaggttgaaggtgaacttgttgtcggagctaactacaaacttgaaattgaccactcacgtcgtcaccgtgttatgaaaaaccacacagcaactcacctcttgcatgctgcccttcacaacatcgttggtaatcacgcagtccaagccggttctcttaacgagcaagaattcttgcgtttcgacttcactcactttgaggcggtaactccagaagaattacgtgctatcgaagaacaggtcaacgaagaaatctggaaagcaactccagtaacaactattgaaacagatattgacactgctaagtcaatgggagctatggccctctttggtgaaaaatatggtaagcgtgttcgtgtcgtttcaataggtgactactctgttgagctttgtggtggtactcacgttgccaacacagctgaaatcggtatgttcaagattgttaaagaagaaggtatcggttcaggaacacgtcgtatcttggcagttacatcacgtgaagcttaccttgcttatcgtgaagaagaagatgccctcaaatcaatcgcagcaacacttaaggctccacaattgaaagaagtgcctaacaaggttgctagccttcaagagcaattgcatgcccttcaaaaagaaaatgctactcttaaagaaaaagcagctgccgctgccgctggtgacgtcttcaaagatgttaaagaagctaacggtgttcgttacattgctagccaagtagaagtttctgatgctggtgcccttcgtacttttgctgaccaatggaaacaagcagactactcagatgttcttgtccttgcggctcacatcagagaaaaagttaatgtccttgtagcaagtaagtctgaaaacgttcacgcaggtaatcttatcaaggttcttgcaccaatcgtatcaggtcgtggtggtggtaaaccagatatggctatggccggtggtagcgatgctaacagtatccaagatcttctctcagcagtagcagaacagttttaa

SEQ ID NO: 86 - protein, 872 aa, synthetic construct
MKQLTSAQIRQMWLDFWKSKGHAVEPSANLVPVNDPTLLWINSGVATLKKYFDGSVIPENPRITNSQKAIRTNDIENVGKTARHHTMFEMLGNFSVGDYFRDEAIEWGYELLTSPEWFDLPKDKLYMTYYPDDKDSYNRWIACGVEPSHLIPIEDNFWEIGAGPSGPDTEIFFDRGEEFDPDNIGIRLLEEDIENDRYIEIWNIVLSQFNADPAVPRSEYKELPNKNIDTGAGLERLAAVMQGAKTNFETDLFIPIIREIEKMSGKAYDPDGETLSFKVIADHIRSLAFAIGDGALPGNEGRGYVLRRLLRRAVMHGRRLGISDAFLYKLVPTVGQIMESYYPEVLEKKDFIEKIVKREEETFARTIDAGSSMLDELLANLKKSGKDTLEGKDIFKLYDTYGFPVELTEELAEDEGFKIDHEGFKAAMKEQQDRARASVVKGGSMGMQNETLANITEPSEFLYEAETAESRLSVIVADDARHDSVNSGQALLVFEQTPFYAEMGGQVADHGTISDAAGTTVARVVDVQRAPNGQALHTVEVEGELVVGANYKLEIDHSRRHRVMKNHTATHLLHAALHNIVGNHAVQAGSLNEQEFLRFDFTHFEAVTPEELRAIEEQVNEEIWKATPVTTIETDIDTAKSMGAMALFGEKYGKRVRVVSIGDYSVELCGGTHVANTAEIGMFKIVKEEGIGSGTRRILAVTSREAYLAYREEEDALKSIAATLKAPQLKEVPNKVASLQEQLHALQKENATLKEKAAAAAAGDVFKDVKEANGVRYIASQVEVSDAGALRTFADQWKQADYSDVLVLAAHIREKVNVLVASKSENVHAGNLIKVLAPIVSGRGGGKPDMAMAGGSDANSIQDLLSAVAEQF

SEQ ID NO: 87 - DNA, 1092 bp, synthetic construct; identical to SEQ ID NO: 11
  source 1..1092: mol_type = other DNA

SEQ ID NO: 88 - protein, 363 aa, synthetic construct; identical to SEQ ID NO: 12

SEQ ID NO: 89 - DNA, 675 bp, synthetic construct
  source 1..675: mol_type = other DNA
atgtcaaatctaattttcgactcagtcacaaaaatttttggtaaagggagtagtaagtacgtagcactagaaaatattaatttcgaagcagaaagcggtcagcttattttagttgtagggccatcaggatcagggaaaactacatttttgactatagcaggcggactacagacaccaactaatggtgacgtaaaaataaatgacagtacaattaacagtttgtccaaaaaacaacaaacaaaattaagattggaaaaaataggatttattttacaatcttataacttagtcccatttttaacggtcgaagaacagtttaaattcgtagataaactaaaaaaacaaaatttgactgaacaaaaattgcatgagcttctttctgatttaggacttttagaactgctaaaaaaatatcctaatcaactttctggtggacaaaagcagcgtgtggcaattgctagagcattatacactgatcctgactatattttagcagatgaacccacagccgcattagatacagatagatccatgaaagtaatcgagttactcagagatttagcgcacaaaagaaataaaattattattgtagttactcatgacttaaggttaaaagatatggcagacaaagtttatcagataattgacggtaaaatgacaatcctggagaagtaa

SEQ ID NO: 90 - protein, 224 aa, synthetic construct
MSNLIFDSVTKIFGKGSSKYVALENINFEAESGQLILVVGPSGSGKTTFLTIAGGLQTPTNGDVKINDSTINSLSKKQQTKLRLEKIGFILQSYNLVPFLTVEEQFKFVDKLKKQNLTEQKLHELLSDLGLLELLKKYPNQLSGGQKQRVAIARALYTDPDYILADEPTAALDTDRSMKVIELLRDLAHKRNKIIIVVTHDLRLKDMADKVYQIIDGKMTILEK

SEQ ID NO: 91 - DNA, 1008 bp, synthetic construct
  source 1..1008: mol_type = other DNA
atgacaaatcgtaaagacgatcacatccgttatgccctcaagtaccagtcgccctacaatagttttgacgatatggaacttatccacaagtctctaccgacttatgatttagatcagattgacttgtccacacattttgctggacgagattggaaattccccttttatatcaatgccatgacaggtggatctgccaaaggtggggctgttaatcgaaaactagctgaggtggctagccgaacaggtattctcatggtgacgggttcctacagtgctgctctaaaaggagaggcaccagaatcctttgactatcgtaatgagtttccagacttagaccttgcgaccaatatcggtgttgataagtctgtggacctggggattaagacggtggaagctatggatccagtctttttacagctccatgttaatcttatgcaggagcttctcatgcctgagggagagcgtattttccacacttggaaggaaaatgtggcggcttatgcacagaaaatagaggttcctcttgtgcttaaggaggttggctttggtatggatgagaaaaccattcgctacgccatgtctcagggcatcaagactgtggacatctcaggtcgtggtggaactagctttgcctatattgaaaatagtcgaggtggtaactgtgattacctaaatgactggggacaaagtacggttcagacccttttgcaagcccaagaccttcgtgaggaggtggaaatcctagcctctggtggcgtgcgcaatccacttgatatggttaaatgtctggtcttaggagcaaaaggtgtaggcctctcacgaacagttcttgaattggtggaacgttatcctgtggataaggttgtggctatcgtcaatggctggaaggatgaccttcgccttatcatgtgtgcccttgattgtcgcactattgatgagcttaaatctgtagattacatcttatatggaaagttacaagggacttatgtcaaacaagagaagtcataa

SEQ ID NO: 92 - protein, 335 aa, synthetic construct; identical to SEQ ID NO: 4

SEQ ID NO: 93 - DNA, 798 bp, synthetic construct
  source 1..798: mol_type = other DNA
atgaagaaattttggtttatgcttacagccctagttgccacgtgttttcttgttgcatgtagggggagtcaaggagtgagcacaaaaacgattaaggaaaataagaaaattgtcgtagcgacagagtctgaatttgcaccatttgaattcaaatcgttggtcaatggcaaggataccttagttggagcagacatcgagttggctaaagctatcgggaaaaaacttggtgtgaaagttgaattttctgttatgtcttttgacaatgtccttgctagtgtgcaaagtggtaaagcagacattgccattgcagggatctcggtaacagacgagcgtaaaaaagtctatgatttttctgatagctactatacctctgaaaatgtagtgattgtcaaaaaagacaaggtaaatgattttacaagtgctgatagtctcgctaaacaaactgtcggtacgcaaaaagggtctgttcaagaaacagttgctaagaaacaattctctaaggcaagtgtggtttcactcgcttcaaatggcgagatgattaacgaactcaagtcaggacagattcaagcggttgtccttgaaaaagctattgcagaaggttatatcgcacaaaatgatgatttaacccttgccagctttaaccttaagtcagatggttcagatgcttatgcagtcgcgatacgtaagggttcggacgatttacttaaagaaatcaatgctgtcatcaaagagaccaaggagtctggaaaatttgaccaatggttaaaagaagcatcaagttacaaacaaagtaagtaa

SEQ ID NO: 94 - protein, 265 aa, synthetic construct
MKKFWFMLTALVATCFLVACRGSQGVSTKTIKENKKIVVATESEFAPFEFKSLVNGKDTLVGADIELAKAIGKKLGVKVEFSVMSFDNVLASVQSGKADIAIAGISVTDERKKVYDFSDSYYTSENVVIVKKDKVNDFTSADSLAKQTVGTQKGSVQETVAKKQFSKASVVSLASNGEMINELKSGQIQAVVLEKAIAEGYIAQNDDLTLASFNLKSDGSDAYAVAIRKGSDDLLKEINAVIKETKESGKFDQWLKEASSYKQSK

SEQ ID NO: 95 - DNA, 1863 bp, synthetic construct
  source 1..1863: mol_type = other DNA
gtgtcagaagataaaacacaaaatggctatgaagaaagccaagagttagatttccaagatgccaaggaaatgacagtcggtgaggctgtccgcaaagaagctgagattaatgctggtgtaaccgaaacagatagtattttggataagtatattaagcaacatcgtgaagaagttacctcacaaaaattttcaaagaaaattgaagcagatggcgatacatcacccttggatgccttcattcaaaagcaacgtcaggaatttgctgattcaggtttgattggtcaaacattggctaatgagtcaactaactcaacgactgctgacgaaacagtaacacctattacatctggttttggcacaactgaaactaaggcagatgatactcaagctcctgttgactctgaaatcactgtgaaaccagaaagtgaaagctctgaaaccatcataacatcaactaatgctgatcgttttatcacgtcagaagcagaaaaatttgatcttggtgaagcccttactgcgacgactacctcaactaatcaacaagttgataacactgctatggtagacaatgttgatgatccggaaccagaaagtactttacctgctgatgacaagtcttctgaaccacaagcctctaaacctctgttggaagatgttggagtctcagaaacaatcgactcagacgctattgccacaacagttgctggtgtaacaggagttaaagctgatgaagcaaaagcgacaccaaaagtgtctatgacagttagtcgtccatcagcagaagataaaatctcttcagggtatatttctccatctcataaaggtgtctttgatggtgatattccagtttatcgccgtaagggtgttgttatcggtgctctcacagttcttgctctagctataattggtgggagctatgctctttataaagtaacacatagccaatctgcgaaaactacaagcactgcttcttcagcagtgatatcatcatcaagcaagggcgcatcagcttctgataataaagccttcgaagacatgtataagaatttcttcacagatgatgagcaaacaaaactagctaatgatcaatttggtaagctatcagacttggaaaaactacttaaaaaattggaaaaaaccaagtattatgatgcagctaagaccaaatacgataacttgaagaaacaaatcgaagctattgaaaaagtcaatagtaagtatgaatcagatgctcttgtagacggtagctataatgcctctatctcagttaaatctgatgcaaactttaatgatctttcagaacgtgtaacaaatactggcaatgctagtcttgatagcattatccaagaagtcattaagggcggtaaaacacagcttgaagaaaagggtaaggctgcaagtgcaacgtctgcagtgacagcttctgaaagtgttaataccgctactccttcgggagacaacacatctggagcagctaattcaggtgtaacaggtgcggctggtggagtttctagtggtacagctgcaacttcaacagttgtgacacgtggcatcatgaattacaatccatctatccttcaacgtgatcgctctcgtgtgccttataatgcaaacgtagtagcggatacaagtaatccagcttggacttgggctgatggtgttcttgataagatcattgcaacatctcactcacgtgggtacttctcaggagataactttatccttgagccagtcaacatcatcaacggaaatggttattacaacatgtaccttccagatggaacttatctctttagtatcaactgtaagacaggttactatgtaggcaatggttcaggtcactcagataaacttgattattaa

SEQ ID NO: 96 - protein, 620 aa, synthetic construct; identical to SEQ ID NO: 6

SEQ ID NO: 97 - DNA, 1968 bp, synthetic construct
  source 1..1968: mol_type = other DNA
atgaataataaaaataataatagcttcttacggaatgctttcctttacatcgtaatgattattgcagttgtaacggcagtacagtattttgttggagaaagtcaaagtccaagtcaacaattatcttatacccagttgattaagaaaattgaagatggtaaggtaaaatcaattacctatcaacctaatggtagtatcattgaagttaaaggagactacaaaaaggctgaaaaggttgataatgacctaagtttgccatttttaggtggagcatcgtcagagacacaaagttttacatcaactgtgcttcataatgaaacgataatacaggcattacagtcagcttcagaaaaagctaatgttaatatcaaaatgatacgtgaaagttcaagtggtacttggatttcgtatctctttagttaccttcctttgattggtattgctatttttttcatttttatgatgaatcaaggtggtaatcgcggtgccatgaattttggccgtaatcgtgctaaagctcagtcaaaagaaaatattaaagttcgctttacagatgtagccggtgcagaagaagaaaaagaagaacttgtagaaatagtagatttcctcaaaaatccacgtaaatataaagctttgggtgcacgtattcctaaaggagtcctcctcgaaggccctccaggaacaggtaaaaccctccttgctaaagctgtggctggtgaagctgatgtaccattctttagcatctcaggttctgattttgttgaaatgtttgtcggtgtcggagctagccgtgtacgtagtctgtttgaagatgctaaaaaggcagagcgtgctattatctttattgatgaaattgatgctgttggtcgtcgtcgtggtaccggaatgggaggtggcaatgatgagcgtgaacaaacccttaaccaattgttgatagagatggatgggtttgatggtaatgagaatattattgtcattgctgcaaccaaccgtagtgatattcttgatccagcccttctacgtccgggacgttttgaccgtaaagttcttgttggacgccccgatgtaaaaggtcgtgaagccatcttgaaagtacatgctaagaataaaccattggctgaagatgtcaaccttaaggttgttgctcagcaaactccaggttttgttggagctgacttagagaatgtcttgaatgaggctgctttagttgctgcacgacgtggtaagactaagattgatgctagtgatattgatgaagcagaggatcgtgtcattgctggaccatctaagaaagatcgccaagtttcggaacatgaacgtaaagttgttgcctaccatgaggctggtcacacgattgttggattaacactctctagtgctcgtgatgttcacaaagttactattgttccccgaggtcgtgcaggtggctatatgatttctttaccaaaagaagataaaatgttgtcatctaaagacgaactgaaagaacaattagcgggattgatgggaggacgcgttgcagaagaaattgtttttaatcttcaaacttctggtgcttccaacgactttgagcaggcaacacagttagcacgtgcaatggttactgaatatggtatgagtgaaaaacttggaccagtccaatatgagggtaaccacgcgatgaacccaggtcaatttacgccagacaagtcttactcagctcatacagctcaactcatcgacgaagaaattcgatcacttttagttgaggcacacgacagagcagctgaaattatcaatgcaaaccgagacacacatgctcttattgcagaagctttgcttaagtatgaaacacttgacgctgcacaaattaaatcaatttatgagactggtaagatgccggtagattcagaagaagataaccatgctttatcttttgatgaagttaaaaagagacttgaaaacaaaaataaagatgaagaagagtag

SEQ ID NO: 98 - protein, 655 aa, synthetic construct
MNNKNNNSFLRNAFLYIVMIIAVVTAVQYFVGESQSPSQQLSYTQLIKKIEDGKVKSITYQPNGSIIEVKGDYKKAEKVDNDLSLPFLGGASSETQSFTSTVLHNETIIQALQSASEKANVNIKMIRESSSGTWISYLFSYLPLIGIAIFFIFMMNQGGNRGAMNFGRNRAKAQSKENIKVRFTDVAGAEEEKEELVEIVDFLKNPRKYKALGARIPKGVLLEGPPGTGKTLLAKAVAGEADVPFFSISGSDFVEMFVGVGASRVRSLFEDAKKAERAIIFIDEIDAVGRRRGTGMGGGNDEREQTLNQLLIEMDGFDGNENIIVIAATNRSDILDPALLRPGRFDRKVLVGRPDVKGREAILKVHAKNKPLAEDVNLKVVAQQTPGFVGADLENVLNEAALVAARRGKTKIDASDIDEAEDRVIAGPSKKDRQVSEHERKVVAYHEAGHTIVGLTLSSARDVHKVTIVPRGRAGGYMISLPKEDKMLSSKDELKEQLAGLMGGRVAEEIVFNLQTSGASNDFEQATQLARAMVTEYGMSEKLGPVQYEGNHAMNPGQFTPDKSYSAHTAQLIDEEIRSLLVEAHDRAAEIINANRDTHALIAEALLKYETLDAAQIKSIYETGKMPVDSEEDNHALSFDEVKKRLENKNKDEEE

SEQ ID NO: 99 - DNA, 1968 bp, synthetic construct; identical to SEQ ID NO: 97
  source 1..1968: mol_type = other DNA

SEQ ID NO: 100 - protein, 655 aa, synthetic construct; identical to SEQ ID NO: 98

SEQ ID NO: 101 - DNA, 1968 bp, synthetic construct
  source 1..1968: mol_type = other DNA
atgaaacttaaaaaaattcttggaattacgggtgtagcccttctaagtgtaggtgtgttagcggcttgttcatctaaatcaaacactagcggaactacttatcgtaacgtttacagttcagatccagaaaccttggattatattacatcaattatgggagggacaaaagctgttttgactaatggtgtagatggtcttatggaagcggacaaatatggtaatcttgttccatcagtagctgaagactggtctgtgtctaaagatggtcttacttatacctataaaattcgtaaagggatcaaatggtatacttctgaaggtgaagagtacgctaatgttactgctaaggactttgtaacgggtcttaagcatgccgctgatgccaaatctggagccctttatttggtacaagattctattgctggattgtctgactacctttctggtgccaataaagatttctcaaatgttggcgttaaagcaatagacgattacactctgcaatacacactaaaaaaacaggaaccatactggaactctaagacaacctatggtgtgctcttcccagttaacgaagactttcttaagaacaaagggaaagattttggtaagtcaacagacccaacctctatcctatataacggtcctttccttcttaaatctttgacagctaaatcatctatcgagttgactaagaatgagaattattgggataagaaaaacgtgcattttgactctatcaaattctcatattctgatggatcggatctagaatcaatagaacgtagtttctctgacggcgctctcagcattgcacgtgtcttccctacgagttcaaactatgcatcggtagaaaaaaaatataaagataatattttttacacagaaccaggtgcatctacatcggttattggtgtaaacattgaccgccaaagctataaattctcagctaagaagacagatgctgaaaagacatcaactaagaaggctcttctaaacaaagactttcgtcaatcaatcaacttcgctattgatcgtacagcctatcaatcacaagtaaacggtaaagatggcgcagctcttgctctacgtaacctttttgttccttatgattttgtcagtgcaggcgataagacctttggtgatcttgtgacagaaaaaatgagctcttatggtgatgagtggtctggtgttaactttgcagatggtcaagagggtctctacaatgctgaaaaagctaagactgagtttgccaaagccaaagaggctcttcaaggagaaggagtacaattcccagttcacttagaccttccagttgatcaaagttctaaacttaatgttgctcaagctcaatctttgaaacaaaccattgaaaaatcattgggtagcgaaaatgttgttatagatattaaccaaatgtcatctgatgatatgcaaaatgcgactagtaatgcggcaaatgctgcagcagaagactgggatatttctaacggtgtgggttggggtccagactatcaagatccatcaacttatcttgatatcttaaaaacaacatcaagtgaaaataccaaggctttcaatggttatgatgatccaaacaatgcagcagcagcccaagtaggtcttaaagattatgatgccttgattgattcagcagcttcagaaacaacagacctcaacgctcgttacgaccgttatgctcaagcccaagcttggttggaagatagctctcttgtcattccattgactgttagtaacggtgcagcaccgattatctcacgtttgacaccatttacgggtgctagcatacaggtgggtgataagggctcatcctacctcaaatatgtgaaatcacaagaaaaagtagttactaagaaagaatatgaacaaagtcgtgaaaaatggcttaaagaaaggaaagcatctaacgaaaaagctcaaaaagatcttgagaaacacattaaataa

SEQ ID NO: 102 - protein, 655 aa, synthetic construct
MKLKKILGITGVALLSVGVLAACSSKSNTSGTTYRNVYSSDPETLDYITSIMGGTKAVLTNGVDGLMEADKYGNLVPSVAEDWSVSKDGLTYTYKIRKGIKWYTSEGEEYANVTAKDFVTGLKHAADAKSGALYLVQDSIAGLSDYLSGANKDFSNVGVKAIDDYTLQYTLKKQEPYWNSKTTYGVLFPVNEDFLKNKGKDFGKSTDPTSILYNGPFLLKSLTAKSSIELTKNENYWDKKNVHFDSIKFSYSDGSDLESIERSFSDGALSIARVFPTSSNYASVEKKYKDNIFYTEPGASTSVIGVNIDRQSYKFSAKKTDAEKTSTKKALLNKDFRQSINFAIDRTAYQSQVNGKDGAALALRNLFVPYDFVSAGDKTFGDLVTEKMSSYGDEWSGVNFADGQEGLYNAEKAKTEFAKAKEALQGEGVQFPVHLDLPVDQSSKLNVAQAQSLKQTIEKSLGSENVVIDINQMSSDDMQNATSNAANAAAEDWDISNGVGWGPDYQDPSTYLDILKTTSSENTKAFNGYDDPNNAAAAQVGLKDYDALIDSAASETTDLNARYDRYAQAQAWLEDSSLVIPLTVSNGAAPIISRLTPFTGASIQVGDKGSSYLKYVKSQEKVVTKKEYEQSREKWLKERKASNEKAQKDLEKHIK

SEQ ID NO: 103 - DNA, 1383 bp, synthetic construct
  source 1..1383: mol_type = other DNA
atgagcggaaaatcaattttcgataaactttgggaccgccacgtaattacgggtgatgaggggcaaccccaacttatgtacgtggaccaacattatattcatgaagtgactagtcctcaagcttttcaaggtcttcgtgatgctggacgtaaagtaaggcgtccagatctgacttttgggacctttgaccataatgttccaactgtgaatatctttgatattagagatgctatttctaaggcacaaattgataaacttgctgaaaatgtcattgagtttggtattgacaatgcgtcacatggttcggataagcaaggaattgtacatatggttggtccagaaacaggacgtacgcaaccaggtaagtttattgtttgtggggatagtcacacagctacccacggtgcctttggagctatagcctttggtattggaacgagtgaggttgagcatgtctttgctacacagacactttggcaagtcaaacctaaaaagatgttggtagaattcactgggaagcctcagaaaggcatttattcaaaagattacattctcgccttgattgctaaatatggggttgcttatggagtgggttatgttgtagagtaccgcggtgaggcaattgaccgtttgactatggaagaacgtatgacaatctgtaatatgtcaattgaatttggttcgaaaatgggaattatgaatccagaccaaacaacatatgattatatgcgtggtcgagaatgtgttcctgaggattttgatgcagcagtagctgactggaagacactagttagtgatgatgatgcagaatacgataaagtcattcgaatggatgtttctgagttggcaccaatggtcacatggggaactaatccatctatgggggtagattttgatacgcctttccctgaagttcgagatatgaatgatgagcgtgcataccattatatgggtcttcgtccgggtcaaaaagcagaagacattaatcttgggtatatctttattgggtcttgtactaatgctcgcttgagtgacttgcagttggcagccagaattgttaaaggtaagaagatttcccctaatttgacagctattgtggttccaggttctagacctgttaaacgagctgcagagaaaattgggttggataagatatttaaagatgctggttttgaatggagagagccaggatgctcaatgtgtcttggaatgaatccagataaagtgcctgatggggtccattgtgcttcaacaagtaatcgaaattttgaggatcgacaagggtttggagcaaaaacacacctttgtagtcctgcaatggcagcggcagcagctatcgccggacatttcgttgatgttcgccgaatgccagaagtacaataa

SEQ ID NO: 104 - protein, 460 aa, synthetic construct
MSGKSIFDKLWDRHVITGDEGQPQLMYVDQHYIHEVTSPQAFQGLRDAGRKVRRPDLTFGTFDHNVPTVNIFDIRDAISKAQIDKLAENVIEFGIDNASHGSDKQGIVHMVGPETGRTQPGKFIVCGDSHTATHGAFGAIAFGIGTSEVEHVFATQTLWQVKPKKMLVEFTGKPQKGIYSKDYILALIAKYGVAYGVGYVVEYRGEAIDRLTMEERMTICNMSIEFGSKMGIMNPDQTTYDYMRGRECVPEDFDAAVADWKTLVSDDDAEYDKVIRMDVSELAPMVTWGTNPSMGVDFDTPFPEVRDMNDERAYHYMGLRPGQKAEDINLGYIFIGSCTNARLSDLQLAARIVKGKKISPNLTAIVVPGSRPVKRAAEKIGLDKIFKDAGFEWREPGCSMCLGMNPDKVPDGVHCASTSNRNFEDRQGFGAKTHLCSPAMAAAAAIAGHFVDVRRMPEVQ

SEQ ID NO: 105 - DNA, 939 bp, synthetic construct
  source 1..939: mol_type = other DNA
atggtttccactgctacaaaagttggtaactttacgtttgataactgtcttatgaatgctgctggtgtttactgcatgaccaaagaagagctggcagaggttgaagcttcagctgctggttcattcgtcacaaaaacagggacacttgaggcccgtcctggtaatccagaacctcgttatgtcaatgtaccccttggttcaatcaactcaatgggtttgccaaataacggttttgaatattatcttgactatgtcattgaattgcaaaatcaacccaatacaaaaaaccatttcctctcactagtgggactctcacaagaagaaacacacactattcttaaaaaagttcaaaaaagtgactatgagggactcgttgaactcaacctctcatgtcctaacgttccaggcaaacctcaaattgcttacgattttgaaaccacaaccgatatccttactgaagtcttctcttactttacgaaacctcttggcgtgaaattaccaccatactttgatattgttcactttgatcaagcagctgctgttttcaaccaattccctcttgtcttcgttaactgtgtgaactctattggtaatggactttacattgaagatgaatctgttgtcatcaaaccaaaaaatggcttcggtggtattggtggtgattacattaaacctactgctttggcaaacgtccatgccttctaccaacgtttgaaacctgagatccaaattattggtactggtggtgtaaaatctggtcgtgatgcttttgaacacatcctttgtggcgcaagtatggtgcaagtaggtactgcacttcaaaaagaaggtgttactatctttgatcgcatcactaaagaacttaaagaaatcatggcaaaaaaaggttacgaaactcttgaagactttcgtggaaaactgcactatattgacagataa

SEQ ID NO: 106 - protein, 312 aa, synthetic construct
MVSTATKVGNFTFDNCLMNAAGVYCMTKEELAEVEASAAGSFVTKTGTLEARPGNPEPRYVNVPLGSINSMGLPNNGFEYYLDYVIELQNQPNTKNHFLSLVGLSQEETHTILKKVQKSDYEGLVELNLSCPNVPGKPQIAYDFETTTDILTEVFSYFTKPLGVKLPPYFDIVHFDQAAAVFNQFPLVFVNCVNSIGNGLYIEDESVVIKPKNGFGGIGGDYIKPTALANVHAFYQRLKPEIQIIGTGGVKSGRDAFEHILCGASMVQVGTALQKEGVTIFDRITKELKEIMAKKGYETLEDFRGKLHYIDR

SEQ ID NO: 107 - DNA, 1542 bp, synthetic construct
  source 1..1542: mol_type = other DNA
atgcttaggagttatgtaacgaaaaaacatctcaccttctattttctagccatcgtcatcacttggttagaggccattatcacccctgcccttatccagtatatcgtcgctagttttaccaaccgccagttgcatctcctctggcagattttgctctgggggattgggggcaatctggtgcttgtcctgggcttggctgggaagcgctattactatgctcgtttgatgactgactttaaggttgggataaaacgcgctatttttcggacttttttatacagccgtcagattcctgatgaggaggtgctgtctgatctggaaaatgatgtccaacagttggaaaatagctatatagagccgaccgtcattatcgtatcctcgctaggttttacagtcgtttccatcgcatatgccctctggaccaatttctatctggggttattgtttatcatcttttactctgtaccagttttgtgtagcggactgggttcgaagcgtttggataggattgcggagcaaaaatccctggcaaatcagaattatatcgcccgtgtcaacaatatgattgcaggtgttgctcctattcgtcattatcagggccagaatttgtttttcaagagattttctaaagacctagaacaggcgttgcaagaagaagtgtcctacgaaaagcaacggtcactgaacagtttatttatcaacagtatagatgcattttgctctgtggcgcccattgtgattggcggattcatgacctaccagaactacctttctgcggctagttttgtcggcatttatctggtctcccacaacatcggctaccagtttcaagaactgtcctactttatcaatacccgcaagtctgccaagtccctctgcgataagtatcaagacttgtttggaaataagttggctactgacagcatatttgaccaatctgtctttcctatccagttggagcaggtcagtgtggagaaggatggggagaccattctagctccttgtgatctgactatccacgctggggaaaagattgctatcatcggagaaagcggagcgggcaaaaccaccctgctcaacatcctatacggggaaatccatcctagtcaggggcaaattcgttatcaaggacgggaactaaccagctgtcaactctatcaggctggtgcctatatcttgcaatctagtcatgtttttgacgatttatctgtggaggaaaacatcgctctgggacaggaaattgaccagcaaagaatggataacatcttgcaagaaacagggctcctcagcctaaaaggcaaggtacccagcaaccaaactttatcaggcggtgaaaaacagcgccttgaaatcgcccgtgctctctatcacaagcgacagtttatcctagcagatgaggtcaaggctaacctggacgccaaaaatcaagaaaaaatcaaccagctgctcttttcacttcctcaagctgtggtggaggtcattcaccactacaccaaggaagacttgaagcgctatgataaagtgattcatttgaaaaagtag

SEQ ID NO: 108 - protein, 513 aa, synthetic construct
MLRSYVTKKHLTFYFLAIVITWLEAIITPALIQYIVASFTNRQLHLLWQILLWGIGGNLVLVLGLAGKRYYYARLMTDFKVGIKRAIFRTFLYSRQIPDEEVLSDLENDVQQLENSYIEPTVIIVSSLGFTVVSIAYALWTNFYLGLLFIIFYSVPVLCSGLGSKRLDRIAEQKSLANQNYIARVNNMIAGVAPIRHYQGQNLFFKRFSKDLEQALQEEVSYEKQRSLNSLFINSIDAFCSVAPIVIGGFMTYQNYLSAASFVGIYLVSHNIGYQFQELSYFINTRKSAKSLCDKYQDLFGNKLATDSIFDQSVFPIQLEQVSVEKDGETILAPCDLTIHAGEKIAIIGESGAGKTTLLNILYGEIHPSQGQIRYQGRELTSCQLYQAGAYILQSSHVFDDLSVEENIALGQEIDQQRMDNILQETGLLSLKGKVPSNQTLSGGEKQRLEIARALYHKRQFILADEVKANLDAKNQEKINQLLFSLPQAVVEVIHHYTKEDLKRYDKVIHLKK

SEQ ID NO: 109 - DNA, 4395 bp, synthetic construct
  source 1..4395: mol_type = other DNA
atgtctgataaatttaagcttcttttaaaacaaattcattttccacagcatgaggaagcttataatgaaattaaaagtggttctattgaatcagtaaagctatttaaatcaaaacgtcagtggttttttgttttttctttcagaaatctactgtcatatgagactttcactctttttgataatctgcttcattcttcttttgattctttaggggctaaagtttcatatatgattaatgtagaagatatttcttgtgatcaatctttacttgaagcttatttttcatatgctttagatattttaaaatcatcacacttttcaatctattccctattctccaatttggggattgagatttctaataatagtatttctgttaaggctcctgcgcatatccttagagagaatttacatgagagatttatagccctgattgctgatgttctttcgaatgttggtttatctaatgtttctatttcggttcttgaggataaagaagcttcatcctctcttgaagaggcttacgagacaaataagatctccttacaagaagaggcagaatctcaagctagacaagcacttcaatcaattgttcaaagttcgccggtaccaccacctcagaagcatcaggcccaaaattttgctgagaaacaaagtcaacgagttgcaagctttgataaggctgagattactccaatgattgaagtcaattctgaagaaaataggattgtttttgaaggatatatttttgatgtggagcaacgtgaaacgaaaacaggtaggattataataaattttaaggtaactgattatacctctagttttgctatgcaacgttgggtgaaggatagcgaagaacttgttaagttcggtatgattaaaaaaggaaactgggtaagagttcgaggtcgtattgagaataatccttttacacatagtttgactatgaacgtacaagatattaaggaaatttctcatactcctcgtaaggatttgatgcccgagggacagaagcgagttgaatttcatgctcatactaacatgtcaactatggatgctataccaacagttgaagagctcatcgatacagcggctttttggggacatccagcagttgcgataacagatcatgcaaatgttcaaagttttcctcatggctaccataaagctaagaaagctggaattaaggcgatttttggtcttgaagccaatcttgttgaggacaaagttcctattgtttataattccgaaaatttggaattaaaagaagcaacttatgttgtattcgacgtagaaacaacaggtttgtctgctgttcataatgatttgatccagattgcggcatcaaaaatgcataaaggtaatattgttgagcaatttgatgaattcattgatccaggatatccgctttctgcgtttacgactgaattgactggtattactgataatcatgttaaaggtgccaaacctttagtgcaagttttacaggagtttcaagaattctgtaaaggagctgtgctagtcgcccacaatgcgacctttgacgttgggtttatgaatgccaattatgaaagacatcagttaccgacaatctcacaaccggttattgatactttagaatttgcacggaatctttatcctgagtataaacgtcatggtttgggtcctttgacaaagcgttttggcgttgccttggaccaccaccacatggccaactatgatgcggaagcaacaggtcgtttgttgtttattttcattaaagatgtattcgaaaagcatggattgactaatctggaacagttgaacacagaattggtctctgatgattcgtataagaagtctcgtgtcaaacatgcgacgctatatgttcagaatcaaacgggtttgaagaatatctttaaattggtaagcctatcaaatgtatcatattttgagggagtagcccgtattcctcgtaaggttttagatgagtatcgtgagggaatcatagtcggttccgcatgtgcagatggtgaagtttttgatactctcttgtctcatggtattgataaggcagttgaagttgctaaatgctatgattttattgaagttatgccaccagcgatttatgcgccattgattgcaaaggatttaatcaaggatgagggagctatagaacaacttattaaagatttgattgaagttgctaatcgtcttgacaagcctgtgcttgctacgggaaatgtacattatatcaatcctgaagatgccatttatcgtgaaatcattgttcgtgctttgggacaaggagctatgatcaatagaccaattggtaagggggaaaacgctcaacctgctcctctaccggaagctcattttagaacaaccaatgagatgctagatgaatttgcctttttggggaaagacttagcctatgagattgttgtagctaatacacaagctatggcaaatcaaatcgaagaggttgaagtcgtcaaaaaagacttgtatacaccttatattgatcgtgcagaagaacaggttgcagagatgacttatgctaaagcatttgagctttatggtaatcctttgcctgatattattgacttgcggattgagaaagagttgtcctcaattcttggtaatggatttgccgtaatatatctagcatcgcagatgctggtgaaccgttcaaatgaacgtggctacctggttggctctcgggggtctgttggctctagttttgttgcaacaatgattggaattactgaggtaaatcctatgccacctcattatttatgtcctaaatgccaacactcagagtttatcacggatggttcttatggctctggctttgatttgcctgataaggagtgctcagaatgtggaactgagtataaaaaagatggtcaggatattcctttcgagaccttcctcggttttgatggggataaggttcctgatattgatttgaacttctcaggagatgatcagccgtcggctcacttggatgtacgtgatatttttggagagcaatatgccttccgtgctggaacggtaggtactgttgcagatcggacagcttatggttttgtcaagggttacgaacgtgattacaataagttctatcgtgatgctgaggttgatcgtttggctatgggtgttgctggggttaagcgaaatacaggtcagcacccaggtggtatcgttgttattccaaattatatggatgtctatgattttacacctgttcaatatccggctgatgatgtaactgctgcttggcagacaactcactttaacttccacgatatcgatgagaacgtgttgaagctagatattctgggacatgatgatccgactatgattcgtaagttacaggacctatcaggaattgatccaaaggatattcgagcagatgatccggatgtgatgaaacttttttcagggactgaggttttaggggtaacgcctgaacaaattgggacctcaacgggcgtgttgggaatccctgagtttgggactaactttgttcgtggtatggtcgaagagacgcatccgacgacctttgcggaactcttacagttatctggtttatcccatggtacagatgtttggttgggtaatgcacaagatttgattaaggaaggtattgccaccctgaaaacagttatcggttgtcgtgacgatatcatggtatacctaatgcatgcagggttagatcctaagatggcctttaccattatggaacgtgtgcgtaagggaatgtggttgaaaatttctgaagaagagcgaaatggttatattcaagccatgcgtgaaaataatgttccggactggtatattgaatcttgtggtaaaatcaagtacatgttccctaaagcccatgcggctgcctacgttatgatggcccttcgcgtagcttatttcaaggtgcaccatcctatttattactattgtgcttacttctcgattcgagccaaagcatttgagcttaagacgatgtctgcagggctcgatgctgttaaagctcgaatggaagatattaaggagaagcgccaacgaaatgaagctactaatcttgaaaatgacttgtttacaacgcttgaaattgtcaatgaaatgttagaacgtggctttacttttggccagttagatctttataagagtcaggcgactgaatttttgattgaaggagataccttgattcctccgtttatcgcacttgaaggtttgggtgaaaacgttgcgaagcaattggttgctgcgcgtgaagaaggggaattcctctccaaaacagaattgcgtaaacgaggaggcctatcatcaaccttggttgaaagattagatgaaatgggcattctaggaaatatgccagaagataatcagttaagtctttttgatgatttcttttaa

SEQ ID NO: 110 - protein, 1464 aa, synthetic construct
MSDKFKLLLKQIHFPQHEEAYNEIKSGSIESVKLFKSKRQWFFVFSFRNLLSYETFTLFDNLLHSSFDSLGAKVSYMINVEDISCDQSLLEAYFSYALDILKSSHFSIYSLFSNLGIEISNNSISVKAPAHILRENLHERFIALIADVLSNVGLSNVSISVLEDKEASSSLEEAYETNKISLQEEAESQARQALQSIVQSSPVPPPQKHQAQNFAEKQSQRVASFDKAEITPMIEVNSEENRIVFEGYIFDVEQRETKTGRIIINFKVTDYTSSFAMQRWVKDSEELVKFGMIKKGNWVRVRGRIENNPFTHSLTMNVQDIKEISHTPRKDLMPEGQKRVEFHAHTNMSTMDAIPTVEELIDTAAFWGHPAVAITDHANVQSFPHGYHKAKKAGIKAIFGLEANLVEDKVPIVYNSENLELKEATYVVFDVETTGLSAVHNDLIQIAASKMHKGNIVEQFDEFIDPGYPLSAFTTELTGITDNHVKGAKPLVQVLQEFQEFCKGAVLVAHNATFDVGFMNANYERHQLPTISQPVIDTLEFARNLYPEYKRHGLGPLTKRFGVALDHHHMANYDAEATGRLLFIFIKDVFEKHGLTNLEQLNTELVSDDSYKKSRVKHATLYVQNQTGLKNIFKLVSLSNVSYFEGVARIPRKVLDEYREGIIVGSACADGEVFDTLLSHGIDKAVEVAKCYDFIEVMPPAIYAPLIAKDLIKDEGAIEQLIKDLIEVANRLDKPVLATGNVHYINPEDAIYREIIVRALGQGAMINRPIGKGENAQPAPLPEAHFRTTNEMLDEFAFLGKDLAYEIVVANTQAMANQIEEVEVVKKDLYTPYIDRAEEQVAEMTYAKAFELYGNPLPDIIDLRIEKELSSILGNGFAVIYLASQMLVNRSNERGYLVGSRGSVGSSFVATMIGITEVNPMPPHYLCPKCQHSEFITDGSYGSGFDLPDKECSECGTEYKKDGQDIPFETFLGFDGDKVPDIDLNFSGDDQPSAHLDVRDIFGEQYAFRAGTVGTVADRTAYGFVKGYERDYNKFYRDAEVDRLAMGVAGVKRNTGQHPGGIVVIPNYMDVYDFTPVQYPADDVTAAWQTTHFNFHDIDENVLKLDILGHDDPTMIRKLQDLSGIDPKDIRADDPDVMKLFSGTEVLGVTPEQIGTSTGVLGIPEFGTNFVRGMVEETHPTTFAELLQLSGLSHGTDVWLGNAQDLIKEGIATLKTVIGCRDDIMVYLMHAGLDPKMAFTIMERVRKGMWLKISEEERNGYIQAMRENNVPDWYIESCGKIKYMFPKAHAAAYVMMALRVAYFKVHHPIYYYCAYFSIRAKAFELKTMSAGLDAVKARMEDIKEKRQRNEATNLENDLFTTLEIVNEMLERGFTFGQLDLYKSQATEFLIEGDTLIPPFIALEGLGENVAKQLVAAREEGEFLSKTELRKRGGLSSTLVERLDEMGILGNMPEDNQLSLFDDFF

SEQ ID NO: 111 - DNA, 420 bp, synthetic construct
  source 1..420: mol_type = other DNA
atgacaaaacgtgtggtttacgtctcaccagaaacaacagttgcaacagctgtagatattatacgtgacaaaggtttgcgtcgtttgccagtaattgaacacgacaaactggttggtcttattacagaaggtacaatggcggaggcctcaccatcaaaagcaacaagtttatcaatttatgaaatgaactatctccttaacaagacaaaagtaggagatatcatgattaaaaatgtccttactgtttccaaatatgctagccttgaggatgctatctacataatgcttcaaaacaaggttggagttcttccagtagtagataatgaccaaatttctggaattattacagataaagatgtattccatgctttttttggaaatctcaggatatggtcaagacggaatccgtattggacttga

SEQ ID NO: 112 - protein, 139 aa, synthetic construct
MTKRVVYVSPETTVATAVDIIRDKGLRRLPVIEHDKLVGLITEGTMAEASPSKATSLSIYEMNYLLNKTKVGDIMIKNVLTVSKYASLEDAIYIMLQNKVGVLPVVDNDQISGIITDKDVFHAFFGNLRIWSRRNPYWT

SEQ ID NO: 113 - DNA, 792 bp, synthetic construct
  source 1..792: mol_type = other DNA
atggcttacgaaacaattctttatagtgttgaagaggaagtggcaacattaactctcaatcgccctgaagtgcaaaatggatttaacattcaagtttgtgatgagattatggctgcaatcgaggaagcaggggaagatgcatctgtgaagttcttggttatcaatgctaaggggaaagtcttttctgttggtggagaccttgatcaaatgaaaaaagctgttgctgtggatgatgtacaatctttggttcgtattgctgagcaagtgaatgaaatatcttttgctctgaaaaaattgcctaagccagtcatcatggttattgatgggccctgtgcaggtgccgctgctaacttagcagttgcagcagatttcacaattgcatctgaaaaagctaaatttattcaagcttttgtaggtgttggtttggcccctgatgctggtggtctttaccttttgactcgttcaattggtattaatcgtgctactcaaattaccatgacaggagagcctgtgtcagctgagaaagctttagattgggggattgcttacaaggtagtcgattcagaaaaacttgagaaaacagtgagtcaattgattaagaaattaaatcgttcttcagttaattcattcaaagcaattaaagaaatggtttgggaatctgaatttgcaggttgggaaaaatacgctaagctagagcttgatctgcagaaatctcttgcatttacagaggactttaaagaaggtgtacgtgcttatagtgaacgtcgccgccctaaattccaaggaaaataa

SEQ ID NO: 114 - protein, 263 aa, synthetic construct
MAYETILYSVEEEVATLTLNRPEVQNGFNIQVCDEIMAAIEEAGEDASVKFLVINAKGKVFSVGGDLDQMKKAVAVDDVQSLVRIAEQVNEISFALKKLPKPVIMVIDGPCAGAAANLAVAADFTIASEKAKFIQAFVGVGLAPDAGGLYLLTRSIGINRATQITMTGEPVSAEKALDWGIAYKVVDSEKLEKTVSQLIKKLNRSSVNSFKAIKEMVWESEFAGWEKYAKLELDLQKSLAFTEDFKEGVRAYSERRRPKFQGK

SEQ ID NO: 115 - DNA, 708 bp, synthetic construct
  source 1..708: mol_type = other DNA
gtggaaactacaagtaatccaaccgtctcattatttgggattgattttgacttgacgatccttgccatgtctctcttgacagtaattatcgtttttggagtggttttttggtcaagtcgtgatatgaccattaaaccaaagggaaaacaaaatgtactcgagttcatctatgaatttgtgaacaataccatttctcaaagtcttggaaaattcacaaaaaattatagtttgctcttgtttgtaattttcacctttgtgtttacagcaaacaacttaggtttgcttgtatctgttaaaagtgagcattacaatttttggtcttctccaacatcgaatttcggtgttactatcactctttctttgattattaccctcgttagtcatattgaggggattcgtaaaaaagggtttaaaggctatctcaaaggttacctttcaccatacccagctatgctcccaatgaacattttggaacaatttaccaaccttgcatcattggccttgcgtttgtttggtaacatttattctggtgaggtactaattgacttgattcttcaattagtgaaatggtctgtttttgcagcacctatgtcctttgctttgaaccttgtctgggttgccttttcagcttttattgggttcattcaagcctatgtctttattatcctaagttctaactacattggtgataaagtcaacgaagaataa

SEQ ID NO: 116 - protein, 235 aa, synthetic construct
VETTSNPTVSLFGIDFDLTILAMSLLTVIIVFGVVFWSSRDMTIKPKGKQNVLEFIYEFVNNTISQSLGKFTKNYSLLLFVIFTFVFTANNLGLLVSVKSEHYNFWSSPTSNFGVTITLSLIITLVSHIEGIRKKGFKGYLKGYLSPYPAMLPMNILEQFTNLASLALRLFGNIYSGEVLIDLILQLVKWSVFAAPMSFALNLVWVAFSAFIGFIQAYVFIILSSNYIGDKVNEE

SEQ ID NO: 117 - DNA, 321 bp, synthetic construct
  source 1..321: mol_type = other DNA
atggaaaagcgtgaattgtatgataaattcgaagaattatcacaaaatttgatgtcaatgttggcagaggtagaagcgattaaggcgaacttctcagggattttcgaggaaaatacggctctcaagttggagaacgacaagttacgtgagcacttgagtcaagtggttcaagaggagacagggactaaaatgtctcatggcaaggtgaacctcgaggctatttatgatgatggattccacatttgcccagatttttatggtcaacgtcgtgataataatgaagcttgtggattctgtgcagaattgttgtacggagattaa

SEQ ID NO: 118 - protein, 106 aa, synthetic construct
MEKRELYDKFEELSQNLMSMLAEVEAIKANFSGIFEENTALKLENDKLREHLSQVVQEETGTKMSHGKVNLEAIYDDGFHICPDFYGQRRDNNEACGFCAELLYGD

SEQ ID NO: 119 - DNA, 1947 bp, synthetic construct
  source 1..1947: mol_type = other DNA
atgattaacattactttcccagatggcgctgttcgtgaattcgaatctggcgtgacaacctttgaaattgcacaatctatcagtaattctttggcgaaaaaagctcttgctggtaaatttaacggtaaattgattgacacaactcgtgtaatcactgaagatggaagcatcgaaatcgtgacaccagatcacgaagatgcccttccaatcttgcgtcactcagcggcacacttgtttgctcaagcagctcgccgtctcttcccagatattcatttgggtgtaggtccagctatcgaagatggattctactacgatacagacaatcaagctggccaaatctctaacgaagatcttcctcgtatcgaagaagaaatgaagaaaatcgttaaagaaaacttcccatcaatccgtgaagaagtgactaaagacgaggcacgtgaaatcttcaagaacgatccatataaattggaattgattgaagaacactcagaagatgatggtggtttgactatttatcgtcaaggcgaatacgttgacctttgtcgtggtccgcacgtgccatcaactggtcgtatccaaatcttccatcttttgaatgttgctggtgcttactggcgtggaaatagtgataatgcgatgatgcaacgtatctatggtacagcctggttcgataaaaaagatctcaaggcttaccttaaacgccttgaagaagctaaagaacgtgaccaccgtaaacttggtaaagaacttgatttgttcatgatttctcaagaagtcggtcaagggcttcccttctggttgccaaatggtgccacaatccgtcgtgagttggaacgctacattgtagacaaggaagtcgctgcgggctaccaacacgtttacactccaccaattgcatcagttgaactttataaaacttcaggtcactgggatcattaccgtgaagacatgttccctccaatggatatgggagatggggaagaatttgtccttcgtccaatgaactgtccacaccatatcgaagtctacaaacaccatgttcactcttatcgtgaattgccaatccgtatcgctgagatcggtatgatgcaccgttacgaaaaatcaggtgcccttacaggtcttcaacgtgtacgtgagatgtcactgaacgatggacatactttcgttgctccagaacaaatcgaagaagaattcaagaaaattcttcaattgatcattgatgtttatgaagatttcaacttgactgattaccgtttccgtttgtcttatcgtgaccctgcagataagcacaagtactttgacaatgatgaaatgtgggaaaatgctcaacgcatgttgaaagcagctgttgatgacatgggtgttgaatactacgaagctgaaggtgaagcagccttttatggaccaaaattggatatccaagttaagactgctcttggtaaggaagaaactctttctaccatccagttggacttcttgcttccagaacgttttgaccttcactacatcggagctgacggtgaagaacaccgtccagtgatgattcaccgtggggttatctcaactatggaacgctttactgcgatcttgattgagaactacaagggtgccttcccaacatggcttgccccacatcaagtaactcttatcccagtatctaacgaaaaacacgtggactacgcatgggaagtggctaagaaacttcgtgatcgtggtgttcgtgccgaagttgatgaacgtaatgaaaaaatgcagtttaagattcgtgcttctcaaacgcaaaaaattccttaccaattgattgttggtgacaaggaaatgaaagacggaactgttaacgttcgtcgttatggtcaaaaacaaacacacactgagactgtgtcagaatttgtagaaaatatccttgctgatatcgctcgcaaatcacgcccagatgccgaataa

SEQ ID NO: 120 - protein, 648 aa, synthetic construct
MINITFPDGAVREFESGVTTFEIAQSISNSLAKKALAGKFNGKLIDTTRVITEDGSIEIVTPDHEDALPILRHSAAHLFAQAARRLFPDIHLGVGPAIEDGFYYDTDNQAGQISNEDLPRIEEEMKKIVKENFPSIREEVTKDEAREIFKNDPYKLELIEEHSEDDGGLTIYRQGEYVDLCRGPHVPSTGRIQIFHLLNVAGAYWRGNSDNAMMQRIYGTAWFDKKDLKAYLKRLEEAKERDHRKLGKELDLFMISQEVGQGLPFWLPNGATIRRELERYIVDKEVAAGYQHVYTPPIASVELYKTSGHWDHYREDMFPPMDMGDGEEFVLRPMNCPHHIEVYKHHVHSYRELPIRIAEIGMMHRYEKSGALTGLQRVREMSLNDGHTFVAPEQIEEEFKKILQLIIDVYEDFNLTDYRFRLSYRDPADKHKYFDNDEMWENAQRMLKAAVDDMGVEYYEAEGEAAFYGPKLDIQVKTALGKEETLSTIQLDFLLPERFDLHYIGADGEEHRPVMIHRGVISTMERFTAILIENYKGAFPTWLAPHQVTLIPVSNEKHVDYAWEVAKKLRDRGVRAEVDERNEKMQFKIRASQTQKIPYQLIVGDKEMKDGTVNVRRYGQKQTHTETVSEFVENILADIARKSRPDAE

SEQ ID NO: 121 - DNA, 456 bp, synthetic construct
  source 1..456: mol_type = other DNA
atgacaataaatgagtggtcagatgtgatagcaatggctgaagccgtgcaaaataagatggtcagtcctcgagaactagtaaaggatactataagtgaagcggaaaggacgaatactaaaatcaatgctatcgtcagtcaacgttacgagaaagcattggtagaagcagaaaatcgagatttttctgataagcctttttcaggagtactaattttcctgaaggatttaggtcaggagcaagccggggagccttcaacagcaggttctcgcctatttacaagttaccatgctaaagaaacggataactatgtcaagaagcttgaagaactgggcttcattattttagggcgttctagcactcctgagtttggtttcaaaaatatcagtgatgcccagattcatggttctgtcaatctgcctgacaatgtgacacggaatgctggcgggtcaagttga

SEQ ID NO: 122 - protein, 151 aa, synthetic construct
MTINEWSDVIAMAEAVQNKMVSPRELVKDTISEAERTNTKINAIVSQRYEKALVEAENRDFSDKPFSGVLIFLKDLGQEQAGEPSTAGSRLFTSYHAKETDNYVKKLEELGFIILGRSSTPEFGFKNISDAQIHGSVNLPDNVTRNAGGSS

SEQ ID NO: 123 - DNA, 804 bp, synthetic construct
  source 1..804: mol_type = other DNA
atgacattttgcagtatagaaagggttgtcatgattttaattgaagatttaaccgttgtgagtcagcgtgagattgcacctcgtatatttgagatggtgcttaagggggagatggttgcggatatgcagcttggtcagtttgtccacctcaaagtgccagacccaagcaagctccttcgtcgtccgatttccatctcagagattgactacaataaaaaagaggcaaccattgtctatcgtgtggagcgtgagggaacagctatcctttcaaagatggtagccggtcaaactattgataccatgggccctcaaggaaatggttttgatatttccattattgaggctgggcagaaagctctccttgttggtggtggtattggcgtgccacctttggttgagacggctaagcagttgaaggctaagggagtggaagttgtatcagtaattggtttcgccaataagaatgccgttattttggaggataaactcagggcttgtggtgatgtttatgtgacgactgacgacggttcatacggtatcaagggttacgtttcgacagtcattgataactttgactggacaccagatgccgtttacagttgtggagctccaggtatgctcaaatatgttgatagcaagtttgaaaatcatcctcacgcctatgtttctatggaagcccgtatggcttgtggaatgggggcgtgctatgcctgtgtagttcacgttaaaggagaaacggatgctaaaaatcttcgtgtttgcgaggagggaccagtctttcctaccggaaaagtcatcgtctag

SEQ ID NO: 124 - protein, 267 aa, synthetic construct
MTFCSIERVVMILIEDLTVVSQREIAPRIFEMVLKGEMVADMQLGQFVHLKVPDPSKLLRRPISISEIDYNKKEATIVYRVEREGTAILSKMVAGQTIDTMGPQGNGFDISIIEAGQKALLVGGGIGVPPLVETAKQLKAKGVEVVSVIGFANKNAVILEDKLRACGDVYVTTDDGSYGIKGYVSTVIDNFDWTPDAVYSCGAPGMLKYVDSKFENHPHAYVSMEARMACGMGACYACVVHVKGETDAKNLRVCEEGPVFPTGKVIV

SEQ ID NO: 125 - DNA, 1086 bp, synthetic construct
  source 1..1086: mol_type = other DNA
atgacagaaaataaaaatgtaatattatcggctcgcgatatcgtcgtggaatttgacgttcgtgaccgtgttttgacagctattcgaggggtttctcttgatttagttgaaggtgaagttttagccttggttggtgagtctggttcaggaaaatcagtcttgactaaaacttttacagggatgctggaagaaaatggtcgtgtagctagcggatctatcgactaccgtggcaaagacttgactaaatttaagagtcaccaagattgggcagctatccgtggagctaagattgcaactattttccaagatccaatgaccagtcttaacccaattaagactatcggaagtcaaattattgaagttattgttaagcaccaagggaaaacagccaaggaagctaaaaaaatggcaattgattacatggacaaggttggtattccagatgctgaaaaacgtttcaatgaatatcctttccaatactctggtgggatgcgtcaacgtatcgttattgccattgccttggcttgtcgtcctgatgtccttatctgtgacgaaccaacaactgcccttgatgtgaccattcaagctcaaatcattgacctcttgaaatcactcaaagaggagtatggtttctcggtcattttcattacccatgaccttggcgtcgtagcaagtatcgcggacaaggttgctgtcatgtatgctggcgaaatcattgaatacgcaactgttgaggaaattttctatgagccttgtcatccatacacgtggagcttgctttcaagtctgccacaattggctgatgataatggaaaactcttctcaattccagggacgccaccatcactttatacaccagttgttggggatgcctttgccttgcgttcagattatgctttgcagattgattttgaagaaaaagcaccacaattccaagtttcagatactcactgggctaagacctggctcttacatgaggatgcgccaaaagttgataaacctgcggttatccaaaatctacatgaaaaaatcctagccaatatgggatttgcacatttaggagatgaggaggaaggcaatgcctga

SEQ ID NO: 126 - protein, 361 aa, synthetic construct
MTENKNVILSARDIVVEFDVRDRVLTAIRGVSLDLVEGEVLALVGESGSGKSVLTKTFTGMLEENGRVASGSIDYRGKDLTKFKSHQDWAAIRGAKIATIFQDPMTSLNPIKTIGSQIIEVIVKHQGKTAKEAKKMAIDYMDKVGIPDAEKRFNEYPFQYSGGMRQRIVIAIALACRPDVLICDEPTTALDVTIQAQIIDLLKSLKEEYGFSVIFITHDLGVVASIADKVAVMYAGEIIEYATVEEIFYEPCHPYTWSLLSSLPQLADDNGKLFSIPGTPPSLYTPVVGDAFALRSDYALQIDFEEKAPQFQVSDTHWAKTWLLHEDAPKVDKPAVIQNLHEKILANMGFAHLGDEEEGNA

SEQ ID NO: 127 - DNA, 1422 bp, synthetic construct
  source 1..1422: mol_type = other DNA
atggaaaagattttcaaattgaaagaacatggtactgacgtccgcacagaagtgactgccggattgacaactttctttgccatgtcttacatcctctttgttaacccagcgatgttggcccaaacaggaatgcctgcacaaggtgttttcttagcaacaattattggtgcggttgcagggacgttgatgatggcattttatgctaacctaccatacgcccaagcgcctggtatggggcttaatgccttcttcacctacacagtggttttttctttgggttacacttggcaagaggccttagcaatggtctttctatgtggtgttatttcattaatcatcacggtaacgaaagtacgtaagatgattattgagtctatcccaacagctcttaaatctgctatttcagcaggtatcggtgttttccttgcctatgttgggattaaaaatgctggtttcttgaaattctcaattaatccacatacctatactgtagcaggtaaaggtgctgatatggccaaggcttcaattacggcttcatcgtcagctacaccaggtttggttgccttcaataacccaactgtaattgtaggtttgattggtcttgcgattgcgattttctttattgttaaaggtattcgtggaggtgttctcctttcaatccttgtaacgactatcattgctatttttgctggtgtcgttgatcttggaaacattgactggcacgcagctagcttgacatcatctattaaagatcttggtaaggtctttggtgcagcgcttggatctaagggattgggattgctctttgcagatgtttctcgtttgcctggtgtctttatggctattcttgccttttcattgactgatatcttcgatactatcggtaccttgattggtactggtgagaaggttggtatcattgcttcaacaggtgataacaacgaatcaaaagctcttgaccgtgctctttactctgaccttgtcggtacaagtattggtgccattgcagggacttcaaatgttacaacttacgttgaatctgcagcaggtattggtgctggtggacgtactggtttgacagctcttgtggttgctgtactctttgccatctctagtttctttagtccagtggtatcaatcgttcctaatgctgccacagcgcctatccttattattgtaggtgttatgatgctctctaaccttaagagtgtcgattgggaagacttgtctgaagctattcctgccttcttcacttctattttcatgggattctcatatagcattacctatggtatcgcagctggtttccttacttatactttggtcaaagtcgtcaaaggtcaagctaaggatgtccactttattatgtggattttggatatcttgtttatccttaacttcatcagtatggctgtgttctaa

SEQ ID NO: 128 - protein, 473 aa, synthetic construct
MEKIFKLKEHGTDVRTEVTAGLTTFFAMSYILFVNPAMLAQTGMPAQGVFLATIIGAVAGTLMMAFYANLPYAQAPGMGLNAFFTYTVVFSLGYTWQEALAMVFLCGVISLIITVTKVRKMIIESIPTALKSAISAGIGVFLAYVGIKNAGFLKFSINPHTYTVAGKGADMAKASITASSSATPGLVAFNNPTVIVGLIGLAIAIFFIVKGIRGGVLLSILVTTIIAIFAGVVDLGNIDWHAASLTSSIKDLGKVFGAALGSKGLGLLFADVSRLPGVFMAILAFSLTDIFDTIGTLIGTGEKVGIIASTGDNNESKALDRALYSDLVGTSIGAIAGTSNVTTYVESAAGIGAGGRTGLTALVVAVLFAISSFFSPVVSIVPNAATAPILIIVGVMMLSNLKSVDWEDLSEAIPAFFTSIFMGFSYSITYGIAAGFLTYTLVKVVKGQAKDVHFIMWILDILFILNFISMAVF

SEQ ID NO: 129 - DNA, 1131 bp, synthetic construct
  source 1..1131: mol_type = other DNA
atgacaaaattattttcaaattataaacgggcagcgattgattttgcttcggctcaaggcaattatttgacagataccactggcaagacctatttggatttttcatctggtatcggggtgactaatctttgctatcatccccgtgtcaatcaagctctaacagatcaggtaggcaagattttgcaccagcctaacctttattacaatcagttgcaagaagatgttgccggtcttttaactggtgacaaggactatctggcctttttctgtaacagtggtgcagaagccaatgaggcagccataaaaatcgcccgtaaggcatcgggtaaacaagaaatcattaccttccaaaattccttccacggtcgaaccttcggatccatgtctgccactggtcaggataaaatcaaacaaggttttggtgaaggcgtgccccactttagctatgccatttttaacgacatggacagtgttaaggctctagccaataaagaaacgtcggccattatgcttgagttggttcaaggagaatcaggtgttcaaccagcggacaaggcctttgtcaaggctttgtctgattactgtcaggaaactggtatttatctcatcgtagacgaagttcagactggtattggtcgtactggcaagctctttgcttatgaacattacgatattgagccagacatctttaccctggccaaaggtttggcaaatggtgtaccagttggtgccatgcttgctaagtcatctcttggaggggctttctcttatggaagtcacggctctacttttggtggcaataaactatctatggcagcggccaaggctacgcttgaagtcatgttgactccaggttttctggatactgcccttgaaaatgggaacaagctacaagttaaattgcaagaggtcttgtctgataaagagacagtgaccactgtgcgtggattgggctacatgattggaattgagactactggaaatctgagtgaactggtacaagctgctagagataagggattaattgtcttaactgcagggacaaatgttatccgtcttctgcctccaatcactctaagtgatgctgaaatcgaaaaaggtgtggctatcctatcagaaatattttactag

SEQ ID NO: 130 - protein, 376 aa, synthetic construct
MTKLFSNYKRAAIDFASAQGNYLTDTTGKTYLDFSSGIGVTNLCYHPRVNQALTDQVGKILHQPNLYYNQLQEDVAGLLTGDKDYLAFFCNSGAEANEAAIKIARKASGKQEIITFQNSFHGRTFGSMSATGQDKIKQGFGEGVPHFSYAIFNDMDSVKALANKETSAIMLELVQGESGVQPADKAFVKALSDYCQETGIYLIVDEVQTGIGRTGKLFAYEHYDIEPDIFTLAKGLANGVPVGAMLAKSSLGGAFSYGSHGSTFGGNKLSMAAAKATLEVMLTPGFLDTALENGNKLQVKLQEVLSDKETVTTVRGLGYMIGIETTGNLSELVQAARDKGLIVLTAGTNVIRLLPPITLSDAEIEKGVAILSEIFY

SEQ ID NO: 131 - DNA, 1038 bp, synthetic construct
  source 1..1038: mol_type = other DNA
atgacaaaaaaaattgtaacactttctggtgatggtattggacctgaaatcatggcagcaggtcttggagttctggataaagtagcttcaaaaattgaatttgattatgatgttgatgctaaaccttttggtggtgctggtattgatgctgagggtcatccacttcctaaagctactctagaagctgctaaatcggcagatgctatccttttggctgctattggcggacctaaatatgataatgcaccagttcgtcccgaacaagggcttttggccattcgtaaggagttgaatctctttgctaatatccgtcctgttcgtatctttgatgctttgaaacacttgtcacctcttaaaccggagcgtattgaaggcgtagattttgtcgttgttcgtgagcttacaggtggtatttactttggtgagcacattttagaggaagataaggctcgtgatataaatgattattcagctgatgagattcgtcgcatcatgcgtcgtgccttcaagattgcccaaggtcgtggcaaaaaggttactagcattgataaacaaaatgttttggcgacctctaaattgtggcgtaaggttgctgatgaagtttctttggagtttccagatgtaactcttgaacaccagctcgttgatagtgcagcaatgattatgattaccaacccagctcgctttgatgttgttgtgactgaaaacctctttggtgatatcttatctgatgagtcaagtgttcttccaggtaccctaggtgttatgccgtctgctagtcattctgaaaacggtccaagtctttatgaaccgattcatggttcagcaccagatatcgctggtcaaggtattgccaatccaatttcaatgattttgtcagtagctatgatgttacgtgaaagttttaatgaaactgaaggggcagaattgattgaaaatgctgttgataagaccttgaatcaaggcatcttgacacgtgatttgggtggtcaggcttcgactgctgaaatgacagcagctattattagcaatctctaa

SEQ ID NO: 132 - protein, 345 aa, synthetic construct
MTKKIVTLSGDGIGPEIMAAGLGVLDKVASKIEFDYDVDAKPFGGAGIDAEGHPLPKATLEAAKSADAILLAAIGGPKYDNAPVRPEQGLLAIRKELNLFANIRPVRIFDALKHLSPLKPERIEGVDFVVVRELTGGIYFGEHILEEDKARDINDYSADEIRRIMRRAFKIAQGRGKKVTSIDKQNVLATSKLWRKVADEVSLEFPDVTLEHQLVDSAAMIMITNPARFDVVVTENLFGDILSDESSVLPGTLGVMPSASHSENGPSLYEPIHGSAPDIAGQGIANPISMILSVAMMLRESFNETEGAELIENAVDKTLNQGILTRDLGGQASTAEMTAAIISNL

SEQ ID NO: 133 - DNA, 732 bp, synthetic construct
  source 1..732: mol_type = other DNA
atgcgtctggacaagtttttggtagactgtggtgtcgggagtcgtagtcaagtcaagacctttctgaaaaagaagcaagtgaccgtaaatgggcaggtggagacatcacctaagactcagattgatgaggataaagaccaaattgcatacttgggtcaggagttgacacatgagacctttgtttactatctcttaaataaaccgcaaggtgtcatttcagcaacggaagactctcgtcataagactgtcctcgatttactcgatgagacagcaagacataagcaggtttttccagttggtcgcttggatattgatactcatggtcttcttctattgaccaataatggagacttagctcacgctatgctgtctcccaagaagcatgtggacaagatttatcaggccaaggtggctggtattatggatgaagaggatatcttggcttttgaaaaagggatcaagcttaaagaccatacttgtcagccagcgaaattagagattgtatcagtcgacaggaatgcgagtactagtttggttcaaattaccattgccgagggaaaattccatcaggttaagcgtatggtagccgcttgcggtaaggaagtggcggatttacaacgtttgaaaatgggacctttaagcctgccaaatgatttagagcttggtgattggcgacgtttgacacaggaggagttagacgctctaactatttttggaattcctttggcctaa

SEQ ID NO: 134 - protein, 243 aa, synthetic construct
MRLDKFLVDCGVGSRSQVKTFLKKKQVTVNGQVETSPKTQIDEDKDQIAYLGQELTHETFVYYLLNKPQGVISATEDSRHKTVLDLLDETARHKQVFPVGRLDIDTHGLLLLTNNGDLAHAMLSPKKHVDKIYQAKVAGIMDEEDILAFEKGIKLKDHTCQPAKLEIVSVDRNASTSLVQITIAEGKFHQVKRMVAACGKEVADLQRLKMGPLSLPNDLELGDWRRLTQEELDALTIFGIPLA

SEQ ID NO: 135 - DNA, 1440 bp, synthetic construct
  source 1..1440: mol_type = other DNA
atgacatacgaagttaaatctcttaatgaagaatgtggtgtttttggaatttggggtcacccagatgctgccaaattaacttatttcggactttatagtcttcaacaccgtggccaagagggggcaggaattctctcaaatgatgcgggtcaattgaagcgtcatcgtgatatggggcttctctcagaagttttccgtgatccagccaacttagacaagctgactggtaaggctgctatcggtcatgttcgttatgcgactgccggtgaggcttctgtagacaatattcaaccattcatgtttaagtttcacgatggacagcttggtcttgctcataatgggaatttgactaatgcagtgtcactaagacgtgagttggagaagaatggggccatttttagctcaacttctgattcagaaatcttagctcacttgattcgtcgtagccataacccatcatttatgggaaaagtaaaagaagctctgaacactatcaaaggtggatttgcttaccttcttatgattgaggacaagctaattgcagccttggatccaaatggttttcgtcccctttctattggtaagatggccaatggagctatcgtggtttcatctgaaacctgtgcctttgaagtagttggagcagagtggattcgcgatgtaaatccaggtgaagttgttatcatcgatgataacggcatcacttacgataactatacaacggacactcaattggctgtttgctcaatggagtatatctactttgcccgtcctgattcaaatatccgaggggtcaatgtccatacagctcgtaaacgtatgggtgctcaattagcacgtgagttcaaacatgaagcagatattgtcgttggtgtgcctaattcatcacttagtgcagccatgggatttgcggaagaatcaggcttgccaaatgaaatgggcttgataaaaaaccaatacacccaacgtacctttatccaaccaacacaagaattgcgtgagcaaggagttcgtatgaaactctctgccgtatcaggtgttgttaaaggaaaacgtgtggtcatgattgacgattccattgttcgtggaacaacgtcccgtcgtatcgttcaactcttgaaagaagcaggggcgactgaggttcacgtggctatcggaagtccagcccttgcttacccatgtttctacggtatcgatattcaaacacgtaaggaattgattgctgccaatcacacagtagaagaaatccgtgaaatcattggtgcggatagcttgacttacttgtctatcgatggcttgattgattccattggaattgacacagatgaaccaaatggtggcctttgtgtggactacttcactggcaaatattcaacaccattgtatgactacgaaaaaagctatttggaaagtttggaagaacacacttctttctattaa

SEQ ID NO: 136 - protein, 479 aa, synthetic construct
MTYEVKSLNEECGVFGIWGHPDAAKLTYFGLYSLQHRGQEGAGILSNDAGQLKRHRDMGLLSEVFRDPANLDKLTGKAAIGHVRYATAGEASVDNIQPFMFKFHDGQLGLAHNGNLTNAVSLRRELEKNGAIFSSTSDSEILAHLIRRSHNPSFMGKVKEALNTIKGGFAYLLMIEDKLIAALDPNGFRPLSIGKMANGAIVVSSETCAFEVVGAEWIRDVNPGEVVIIDDNGITYDNYTTDTQLAVCSMEYIYFARPDSNIRGVNVHTARKRMGAQLAREFKHEADIVVGVPNSSLSAAMGFAEESGLPNEMGLIKNQYTQRTFIQPTQELREQGVRMKLSAVSGVVKGKRVVMIDDSIVRGTTSRRIVQLLKEAGATEVHVAIGSPALAYPCFYGIDIQTRKELIAANHTVEEIREIIGADSLTYLSIDGLIDSIGIDTDEPNGGLCVDYFTGKYSTPLYDYEKSYLESLEEHTSFY

SEQ ID NO: 137 - DNA, 1248 bp, synthetic construct
  source 1..1248: mol_type = other DNA
gtgcaggaggttaaaaatgcccgtgtcagtttgacgggtgaaaagtccaaacctactaagttaaaagaagtctcttcaattagttatgcccgcataaaaaccgatatggacgagtttaaccgtgttttaggtggcggtgtggttccggggagtttggttcttattggaggagatccaggaatagggaaatcgaccctcctcttacaggtgtcaacacagctggctaacaagggaactgtcctctatgtttctggggaagaatcggccgagcaaatcaaattgcggagtgaacgtttaggtgatattgataacgaattttatctgtatgctgagaccaatatgcaagcagtccgtagtcaagtggagggtatcaaacccgatttcctcattattgattctattcagaccatcatgagcccagagatctctggcgttcaaggttctgtttcccaggttcgtgaagtaactgccgaactcatacagattgccaaaaccaataacattgccacttttattgttgggcatgtgaccaaggagggacagctggctggaccacgtatgttggagcacatggtggatactgtgctttattttgagggggaacgtcatcatactttccgtattctgagggcagttaaaaaccgttttggttcaaccaatgagattgggatttttgaaatgcagtcaggtggtctggtcgaggtactaaaccctagccaagttttcttagaagaacgcctagacggtgccacaggttctgccattgttgtcacaatggaggggagtcgacctatcctagctgaggttcaagctttggtgacaccgaccgtctttggtaacgcaaagcggacaacgacgggactggactttaaccgtgtcagccttatcatggctgtcttagagaaacgttgcggccttcttctccaaaatcaagatgcctatctcaaatcagctggtggtgtcaagttggacgagccagctattgaccttgcagttgccgttgctatcgcttctagttacaaggagcttccaaccaatccacaagaagccttcgtaggagaaattggcttgaccggagagattcgtcgtgtgactcgtattgagcagcgtatcaatgaagccgctaaactaggttttactaagatttatgttcctaaaaattcgcttcatggcatgaagattccagaaggtatccaggtcattggggtcacaacggtaggagaagtactcaaaaaagtcttttcttaa

SEQ ID NO: 138 - protein, 415 aa, synthetic construct
VQEVKNARVSLTGEKSKPTKLKEVSSISYARIKTDMDEFNRVLGGGVVPGSLVLIGGDPGIGKSTLLLQVSTQLANKGTVLYVSGEESAEQIKLRSERLGDIDNEFYLYAETNMQAVRSQVEGIKPDFLIIDSIQTIMSPEISGVQGSVSQVREVTAELIQIAKTNNIATFIVGHVTKEGQLAGPRMLEHMVDTVLYFEGERHHTFRILRAVKNRFGSTNEIGIFEMQSGGLVEVLNPSQVFLEERLDGATGSAIVVTMEGSRPILAEVQALVTPTVFGNAKRTTTGLDFNRVSLIMAVLEKRCGLLLQNQDAYLKSAGGVKLDEPAIDLAVAVAIASSYKELPTNPQEAFVGEIGLTGEIRRVTRIEQRINEAAKLGFTKIYVPKNSLHGMKIPEGIQVIGVTTVGEVLKKVFS

SEQ ID NO: 139 - DNA, 516 bp, synthetic construct
  source 1..516: mol_type = other DNA
atgttaggtgtcatcctactctatgttggtatggttttgatgagtaatggactccatcgtttagagggtattcctgataaatcaaatgttgtcatgaatatctttacgggtggtctgggtttgattctcaacattattgtcattgcttacggagcttgtacagggcaaggtgctgaatggttttatggtagcgctactggtcttttatttgctttcacctatctttactcagcaatcaatacgattttcgattttgatcaacgtttgtatgggtggtttagtttatttgtggcaattaatacgctaccagcagggattctttgcttaacatctggatatggtggtaatgcttggtatggtattatttggttcttgtggggtgtcctatggctaactgcctttattgaaattaatcttaagaagaatctaggaaaatttgtcccttacctagctatttttgaaggaattgtaacagcttggattccagggcttttgatgctttggggcaagtggtaa

SEQ ID NO: 140 - protein, 171 aa, synthetic construct
MLGVILLYVGMVLMSNGLHRLEGIPDKSNVVMNIFTGGLGLILNIIVIAYGACTGQGAEWFYGSATGLLFAFTYLYSAINTIFDFDQRLYGWFSLFVAINTLPAGILCLTSGYGGNAWYGIIWFLWGVLWLTAFIEINLKKNLGKFVPYLAIFEGIVTAWIPGLLMLWGKW

SEQ ID NO: 141 - DNA, 912 bp, synthetic construct
  source 1..912: mol_type = other DNA
atgacttttgaaacgctcaccattgaaaatttaacaaaatcatttgataaacaacattttgctaataaccatatcttcttagagctagaagctgcaaaaatcacagctcttataggacataatggtgctggtaaatcaactcttttaaagcaaatgattggttttattaagcctgatcagggagatgttcactacggagatatttcttttatacataaccgtaaacaagctcgaacaatgatgagttatatgtcacaacagtatgcacctcttgaaaagttaactgttgagcaaaatttagagatgattggaagaatgagagggttaagtacctcagaactccaaaaagaaattgagcaccttctagcagatctggaaatagtagaataccgtgataaaaaagggaagaatctttcaggaggcttaaaacggcttacatcttttgccatggcattaattggctctgctgatatcatgcttcttgacgaaccaaccaacgatgttgatcctgtacgtcgtgaaaaacagtggcgattattacaaaaactggcttataaaggacacactattatcattgttacccacaatctactagaagttgaaaaatatgcggataattttgctctttttaaccatggtaaattattaaaatctggtccagtacaccaaatgtcttataaaaatcattatcaaatttctttcaattttgttagtaaagaatttttaagtctattcgaacattttacgatggtcaatggttgtgtttgtcagatggaaattgaagaaagtgaattaacccgaatactcattcaagttaaggaaggtatagaaaaagggcttatttcaaacctttcagttaagatgaaaacattatctatttccgatatttataaagaggagcttgttaactga

SEQ ID NO: 142 - protein, 303 aa, synthetic construct
MTFETLTIENLTKSFDKQHFANNHIFLELEAAKITALIGHNGAGKSTLLKQMIGFIKPDQGDVHYGDISFIHNRKQARTMMSYMSQQYAPLEKLTVEQNLEMIGRMRGLSTSELQKEIEHLLADLEIVEYRDKKGKNLSGGLKRLTSFAMALIGSADIMLLDEPTNDVDPVRREKQWRLLQKLAYKGHTIIIVTHNLLEVEKYADNFALFNHGKLLKSGPVHQMSYKNHYQISFNFVSKEFLSLFEHFTMVNGCVCQMEIEESELTRILIQVKEGIEKGLISNLSVKMKTLSISDIYKEELVN

SEQ ID NO: 143 - DNA, 1146 bp, synthetic construct
  source 1..1146: mol_type = other DNA
atgacagaagtattttacgatcatttagcaaagacgcgtcatcagattcaccaacatccagaggtttctggggaggagcatgagacgacagtctttttgaaagcttatttgaaaaatctaggtgttgagcctttaaattatcccttgaagacaggactcatcgctgagattgggtcaggtcatcctattattgccttgcgtgctgatattgatgccctaccgattaaggaaaagacaggacttccttatgctagcgacaatggtgccatgcatgcttgtggtcatgattttcaccagacctctctgcttggtgctgctcagcttttaaaagaaagagaagcagagcttaaggggactgttcgattaatttttcaaccggctgaggaaaatttccaaggggcctatcaggtcattgaagcgggcggtattgagggtgtgtcagctatcatcggttatcacaataatcctcacttgaaacctggtcagattggccttcgttcaggtgctattttggcaggcgtggagcagtttagggttgatgtcaagggtgtttcgtcccatgcagcccgtcctgatttgggtgtggatacggtcttggttactacgactatcattaataatttacagcaaattgtggctcggacggtatctccctttgagtcagctgtattgtcagttactcatatcgaagttggaaatacctggaatgttcttccagcagccggcttcttcgaaggaacgattcggactttcaatcctaaggttcgggaagatgtcattgctcgctttgaaaaagttgttcaggcgacagcggatcagtttggtgctcaagttgacattacttggggtaattcaccttacgtgacctacaatgaccagacactcacaccactcatctttgaaaattctaaagccttcgcagaagtgattgaaacgctgccatcaacaggcggagaagactttgcggcttatcaaaaggaaataccaggagtctttgcctttgtcggcactaatggtgaggaggatgccccaggctggcatcatgatgatttccttgtgaaagatgaagccttgcctgtagccgttaattactatgtggaaaacgctcttttcctccttgattattttaaggagcaagacgactga

SEQ ID NO: 144 - protein, 381 aa, synthetic construct
MTEVFYDHLAKTRHQIHQHPEVSGEEHETTVFLKAYLKNLGVEPLNYPLKTGLIAEIGSGHPIIALRADIDALPIKEKTGLPYASDNGAMHACGHDFHQTSLLGAAQLLKEREAELKGTVRLIFQPAEENFQGAYQVIEAGGIEGVSAIIGYHNNPHLKPGQIGLRSGAILAGVEQFRVDVKGVSSHAARPDLGVDTVLVTTTIINNLQQIVARTVSPFESAVLSVTHIEVGNTWNVLPAAGFFEGTIRTFNPKVREDVIARFEKVVQATADQFGAQVDITWGNSPYVTYNDQTLTPLIFENSKAFAEVIETLPSTGGEDFAAYQKEIPGVFAFVGTNGEEDAPGWHHDDFLVKDEALPVAVNYYVENALFLLDYFKEQDD

SEQ ID NO: 145 - DNA, 1353 bp, synthetic construct
  source 1..1353: mol_type = other DNA
atgggtaaatattttggaacagacggtgttcgtggagaagcaaacgttgagcttacgcctgaattggcttttaaattaggtcgttttggtggttatgtgcttagtcaacatgaaactggccgtcctaaagtttttgtggctcgtgatacacgtatttctggtgagatgttagaatctgctttggtagcgggacttctctcagtaggtatcgaagtctataaacttggggtacttgcgacaccaggtgtatcatacttggtacgtacagaaaatgctagtgctggtgtaatgatttcagctagtcacaatccagcacttgataatggaatcaagttctttggtggagatggctttaaattagatgatgcacgtgaagcggaaattgaagctctcctagatgccgcagaagatactcttccacgcccatcagctgaaggacttggaacgcttgtcgattatccagaaggtcttcgtaaatacgaaaaattcttggtgactactggtcttgacctcggtggtatgaaggttgccttggatgctgctaacggtgcggctgcggtatctgctcgtaatattttccttgacttgaatgcagaaatcgctgttattggtgatcaacctgatggccttaatatcaatgctggtgtaggttctacacatccagagcaattgcaggccttagttagagaatctggttcagcaatcggtcttgcctttgatggtgacagtgaccgtctcattgccgttgatgaaaatggcgatattgtcgatggtgacaaggtgatgtatatcattggtaaatacttgtctcaaaaaggtgagttagctaaaaacaccattgttacaacagttatgtcaaaccttggtttccacaaggctctcgaccgtgaaggtattaacaaggctgtgactgctgttggtgaccgttacgttgttgaagaaatgcgtaaaaacggttataatcttggtggcgaacagtctggtcacgttattatcatggattacaatacaactggtgatggtcaattgacagctatccaattgactaaagtaatggttgaaacaggtaagagcttgtctgaattggcagcagaagtaaccatttaccctcaaaaattagttaatatccgtgtggaaaatagcatgaaagataaagctatggacgttcctgcaattgccgctatcattgaaaaaatggaagctgagatggctggtaatggacgtatcttggttcgtccaagtggtacagagcctctccttcgtgttatggcagaagcaccaacagatgatgaagtgaactactatgtcgatactattgccgatgttgttcgtgcagaaattggtttggactaa

SEQ ID NO: 146 - protein, 450 aa, synthetic construct
MGKYFGTDGVRGEANVELTPELAFKLGRFGGYVLSQHETGRPKVFVARDTRISGEMLESALVAGLLSVGIEVYKLGVLATPGVSYLVRTENASAGVMISASHNPALDNGIKFFGGDGFKLDDAREAEIEALLDAAEDTLPRPSAEGLGTLVDYPEGLRKYEKFLVTTGLDLGGMKVALDAANGAAAVSARNIFLDLNAEIAVIGDQPDGLNINAGVGSTHPEQLQALVRESGSAIGLAFDGDSDRLIAVDENGDIVDGDKVMYIIGKYLSQKGELAKNTIVTTVMSNLGFHKALDREGINKAVTAVGDRYVVEEMRKNGYNLGGEQSGHVIIMDYNTTGDGQLTAIQLTKVMVETGKSLSELAAEVTIYPQKLVNIRVENSMKDKAMDVPAIAAIIEKMEAEMAGNGRILVRPSGTEPLLRVMAEAPTDDEVNYYVDTIADVVRAEIGLD

SEQ ID NO: 147 - DNA, 2436 bp, synthetic construct
  source 1..2436: mol_type = other DNA
gtgcaagataataatttaattgacgttaatttaacgtctgagatgaaaacgagttttatcgattatgccatgagtgttatcgtatctcgtgcactcccagatgttcgtgacggtttaaaaccggtacaacgtcgtattctttatggaatgaatgagcttggggtgacacctgataagcctcataaaaaatctgcccgtattaccggggatgttatgggtaaatatcacccacacggtgactcatctatctatgaagctatggttcgtatggctcaatggtggagttaccgttacatgttggttgacggccacggaaactttggttctatggatggtgatggcgcagccgctcaacgttacactgaggctcgtatgtctaaaatcgccctggagatgctccgtgatctcaataagaataccgtagattttcaagataactacgatggttctgaacgtgagccaattgttcttccatcccgtattccaaatcttcttgttaatggtgctacagggattgcggttggtatggcgaccaacattccaccacacaatttggctgaaactatagatgccgttaaattgatgatggacaatcctgaagtcacaacccgtgaactgatggaagttcttcccggtccagactttccaactggtggtttggtcatgggaaaatctggtattcaccgtgcttacgaaactggtaaaggctctattgttcttcgttcgcgtaccgaaattgaaacaaccaaatcaggtcgtgagcgcattgtagttactgagtttccttatatggtcaataagactaaggtccatgaacacatcgttcgtttggctcaggaaaaacgtattgaggggattacagcagttcgtgacgagtcttctcgtgagggggtacgttttgtcatcgaagtgcgtcgtgatgcatctgccaatgtgatcttgaacaatttgttcaaattaactcaacttcagacaaacttcagcttcaatatgttggccattgaaaagggtgtgcccaaaatcctttccttgcgtcaaattttggctgactatattgctcaccaaaaagaggttgtggtccgtcgtacacagtttgataaggataaggctgaagcacgtgcccatattttagaaggcttattaattgcgcttgatcatctggatgaagtgattacgattattcgtaacagtcaaactgatgcagaggctcaagccgagttgatggctcgctttgaactcactgaacgtcaaagccaagccattcttgatatgcgtttgcgtcgtttgaccggtttggaacgtgataagattcaaaatgagtacgatgttctccttgccttgattactgatttggctgatattttggcaaaaccagaacgtgttattgctatcatcaaggaagagttggatgaaagtaagcgtaaattcgcggacgcccgtcgtacagagctgatggtcggcgaagttgtttcacttgaagatgaagacctaatcgaagaagaagatgtcgttatcactctttctaacaagggatatatcaaacgtttggctcaagacgaattccgttctcagaaacgtggaggtcgtggagttcaagggacaggtgtgaacgatgacgactttgttcgtgatatcgtttcaacaagcactcatgatcatctttactttatgaccaacaaaggtcgtgtataccgtctcaagggttacgaaattcctgagtatggccgtacagctaagggattaccaattgttaatctcttgaagttggatgaaggtgagacaattcaaacagtgattaatgctaagagcgacgaagcatcagataacaatcaccttgtctttgtaactcgccaaggtttagtgaaacgaactaaagaagcagaatttaaaaatatccgtcaaaatggtttgattgccttgaaactcagagaaggtgatgaacttatcaatgtcttcttgactaatggtaatgaagaaattattattggtactaaatttggttacagtgtgcgtttcaaagaggatactattcgcagtatgagtcgtatggctgctggtgttaaaggggtaacccttcgtgacggtgaccaagtcgttggtgcagcagctattactgatgaccaagaagtcttgattatcacggaaaaaggttacggtaagcgtacttcagctactgaatacccaactaaggggcgtggtggtaaaggtattaagacagctaatatcacagataaaaatggtaaccttgcaggcattgtaacagtttctggtgatgaggatattatggtcataactgatacgggtgttattatccgtactgcagttgctaatatctctcaaactggtcgtgtaactcaaggggttaaggtcatgcgtttagatgatactgctcgtattgtcacctttgctttggttgaacctgaagaaattgattaa

SEQ ID NO: 148 - protein, 811 aa, synthetic construct
VQDNNLIDVNLTSEMKTSFIDYAMSVIVSRALPDVRDGLKPVQRRILYGMNELGVTPDKPHKKSARITGDVMGKYHPHGDSSIYEAMVRMAQWWSYRYMLVDGHGNFGSMDGDGAAAQRYTEARMSKIALEMLRDLNKNTVDFQDNYDGSEREPIVLPSRIPNLLVNGATGIAVGMATNIPPHNLAETIDAVKLMMDNPEVTTRELMEVLPGPDFPTGGLVMGKSGIHRAYETGKGSIVLRSRTEIETTKSGRERIVVTEFPYMVNKTKVHEHIVRLAQEKRIEGITAVRDESSREGVRFVIEVRRDASANVILNNLFKLTQLQTNFSFNMLAIEKGVPKILSLRQILADYIAHQKEVVVRRTQFDKDKAEARAHILEGLLIALDHLDEVITIIRNSQTDAEAQAELMARFELTERQSQAILDMRLRRLTGLERDKIQNEYDVLLALITDLADILAKPERVIAIIKEELDESKRKFADARRTELMVGEVVSLEDEDLIEEEDVVITLSNKGYIKRLAQDEFRSQKRGGRGVQGTGVNDDDFVRDIVSTSTHDHLYFMTNKGRVYRLKGYEIPEYGRTAKGLPIVNLLKLDEGETIQTVINAKSDEASDNNHLVFVTRQGLVKRTKEAEFKNIRQNGLIALKLREGDELINVFLTNGNEEIIIGTKFGYSVRFKEDTIRSMSRMAAGVKGVTLRDGDQVVGAAAITDDQEVLIITEKGYGKRTSATEYPTKGRGGKGIKTANITDKNGNLAGIVTVSGDEDIMVITDTGVIIRTAVANISQTGRVTQGVKVMRLDDTARIVTFALVEPEEID

SEQ ID NO: 149 - DNA, 582 bp, synthetic construct
  source 1..582: mol_type = other DNA
ttgacaaaggttaaaatttgtggactgtcgacaccagaagcagtagccactgcagttaaagcaggtgctgactatattggttttgtgttcgccaaaagcaatcgtcaagtgagtcttgaacaagctcacgagttggctaaaggggtgacaggccagacaaaaaccgttggtgtctttgtttcaccaagccttaaggaactggaagaagcgattagtcaggttcccttagatatcgttcagattcatggaacttttgatgaagatctgatttcaaagatatcagtaccagttattcgagcaattcagatttcggatggtgattctcaagtcaagagccaggcgggttatcttctttttgatgctcctattgccggcagtggtcagaactttgactggcaactcttggctgacaagcagattgaacaggactactttatcgctggtggtttggctgtggataatgtggcagaggccaaggaaaccttccatccctatgccttggatgtgtcctcaggtgtagagacagatggttacaaggatttaaagaagattaaagcatttatagaaagagtgaaagcatga

SEQ ID NO: 150 - protein, 193 aa, synthetic construct
LTKVKICGLSTPEAVATAVKAGADYIGFVFAKSNRQVSLEQAHELAKGVTGQTKTVGVFVSPSLKELEEAISQVPLDIVQIHGTFDEDLISKISVPVIRAIQISDGDSQVKSQAGYLLFDAPIAGSGQNFDWQLLADKQIEQDYFIAGGLAVDNVAEAKETFHPYALDVSSGVETDGYKDLKKIKAFIERVKA

SEQ ID NO: 151 - DNA, 2556 bp, synthetic construct
  source 1..2556: mol_type = other DNA
atgaagcgacttatagagagacttcaacctttaaaaactgccgttaaatctgtcttctttatttctattgcggttcttgttgttgtagagttgattcgtttaaaacgaacgatcactttagaatcgctggagtcagctttatcaggtctgtcaatttggcatcttgctttgatggtagtgataggcttagttgctgtgttacccatgctcttttatgaccttatcttgaataaggagctagagacggattattccaaatcctatatactagaaacgagttgggctgtcaataccattaataacttgtctggttttgccggtttagttgacgtgggattgaggtattccttttattctgaagatgggcatgaaaagtctggtgttaaggcactatcacgtttattgccttacttcatgtcgggcttgtcgctcctaagtggcttggtgtttgctaccttttggtttttccctctaagtcctgacttgagaaaatattggttacttttactaggaatctttttatacctaccttttattttctttgtctcaagtcgcgaaaaattgccttattttgggcaagtccctttgaaaacgagactaagccttattttagtatcgactgctgaatggggcacagctcttgtgagttttctgagtgtggcttatttaatgggtctacatgttcctctttataatctgattcccctatattttctagcggttatcattggtatcttttccatgattccaggtgggattggtagttttgatttgattgtgattagtggtctgacgtctatgggtgtcagcaatgctcttgcggtcagtctactattgctctttcgtctaacctactacatcattccatttttattaggtgttgtcttctttttcaaacacatgggaggtcacatcaatgataaatacttcaatattccttcacgagtatttggtggaagtttccatcttatccttgtttatttgttacgttttttaggaatcttcttgattttatctgccctaatcccagaaagcttggcaaatgtttactttattagccgtttcgatcctattcaggaacagttgatctggcagtttccaagtattttatttggaaccctctttgttttcatgtctcgcctcatcaggcgacgcgttaagggagcttttattgtatccttgctgaccttcgtcttgactttgatttacgtcaatctcaatggtacttcctggaccatgtctttcatgattgtgctttctatgatacttatgttacttataaggaaaagattgaatcgtcactattttgtttatagctgggaagacaaaaccaaggatttcctttttttagcctttaccattttggtcctccttgttctgggtggtagtggtttttggtctcatttgttaccacctaggaaccgagatattcttggacattttgttcatatttggtttgatattttactggcatcggtaatcattgcaaccattgtttggcttgtactgagagttttggcacctcgtaagcattttggtcaaagcatggataatgagcgttttacagcacttcttgagaaatatgggggcgcatcagaaagtgctctagcttatttacatgataaacgcctcttttggtatcgtgtcgatggacaagatcaggtagtttttcaatttgcacaaatcagtaacaagtgtgtgattatgggcaatcctattggtaatgaaaaatattatcgtccggcttgggaagctttcttgaaaaacttatctgattggaatttacaggctttattttatgaggcggatgagagagtgacccttatgcttcatgattatggctttgacttcatgaaatttggagaaaatgccatggttgatttgacgacattttctgttgatggtaagcatggtaagaagttccgtaaaccaaccaatcgtgtggaaaaagcaggcttccagtttaaattacttaatcctccattttcggagactcagatgcaggaaatgaaggcagtctcagatatatggcttaacggtcgtaaggagaaaggtttctctcttggtttctttgatgaagcctatttacaacaagcaccgattgctatcgttgagagtaaagaaggagaaattgtggccttcgctaatatcatgccaacccaaaataaacgggtggcaaccattgatcttatgcgttatgactttaaaaaggctcctgaaggcatcatggactacctttttgtcaaactcttccaatacttcaaagaagaaggcaagcaatactttgatatggggatggctcccctagctaacgtaggtactgaagaagacagtttcatagaggaaaaggttgccaatttagtttatgtttttgctcagcgtttttactcttttagtggattgcaacgttacaaagaaaaattctctccaatttggtctccaaaatacattgtctatccaaaaagaacctggcttttgtttgatatgattgctattttcaggattgataatcgtaaaattgaagataaaatgaagaaaagacgtctttggaagtaa

SEQ ID NO: 152 - protein, 851 aa, synthetic construct
MKRLIERLQPLKTAVKSVFFISIAVLVVVELIRLKRTITLESLESALSGLSIWHLALMVVIGLVAVLPMLFYDLILNKELETDYSKSYILETSWAVNTINNLSGFAGLVDVGLRYSFYSEDGHEKSGVKALSRLLPYFMSGLSLLSGLVFATFWFFPLSPDLRKYWLLLLGIFLYLPFIFFVSSREKLPYFGQVPLKTRLSLILVSTAEWGTALVSFLSVAYLMGLHVPLYNLIPLYFLAVIIGIFSMIPGGIGSFDLIVISGLTSMGVSNALAVSLLLLFRLTYYIIPFLLGVVFFFKHMGGHINDKYFNIPSRVFGGSFHLILVYLLRFLGIFLILSALIPESLANVYFISRFDPIQEQLIWQFPSILFGTLFVFMSRLIRRRVKGAFIVSLLTFVLTLIYVNLNGTSWTMSFMIVLSMILMLLIRKRLNRHYFVYSWEDKTKDFLFLAFTILVLLVLGGSGFWSHLLPPRNRDILGHFVHIWFDILLASVIIATIVWLVLRVLAPRKHFGQSMDNERFTALLEKYGGASESALAYLHDKRLFWYRVDGQDQVVFQFAQISNKCVIMGNPIGNEKYYRPAWEAFLKNLSDWNLQALFYEADERVTLMLHDYGFDFMKFGENAMVDLTTFSVDGKHGKKFRKPTNRVEKAGFQFKLLNPPFSETQMQEMKAVSDIWLNGRKEKGFSLGFFDEAYLQQAPIAIVESKEGEIVAFANIMPTQNKRVATIDLMRYDFKKAPEGIMDYLFVKLFQYFKEEGKQYFDMGMAPLANVGTEEDSFIEEKVANLVYVFAQRFYSFSGLQRYKEKFSPIWSPKYIVYPKRTWLLFDMIAIFRIDNRKIEDKMKKRRLWK

SEQ ID NO: 153 - DNA, 2559 bp, synthetic construct
  source 1..2559: mol_type = other DNA
atggctaaggaaaaaatatctccaggaatgcagcagtatttggatatcaagaaaaattatccagatgcttttttgctgtttcggatgggggatttttacgaattattttacgatgatgctgttaaggctgcccagattttggaaattagcctaactagccgtaataagaatgcagataatcccattcctatggcaggtgtgccttatcattcagctcaagcatacattgatgttttggttgagatgggctacaaggtagccattgctgagcagatggaggatcctaagcaggctgtcggtgtggtaaagcgtgaggttgttcaggttatcacaccaggtacagtggttgacagctctaaacctgatagtgccaacaatttcttggtggccattgacaaggttggaagtcgttttggtctttcctatatggatgtgtcaacgggtgaattttttgcgacagagttggatgattttagttcagtttgtagtgaaattcagaaccttaaggcacgtgaagtagtggttggatatgatctacttgaaactgacgaacaagttttagtgaaccaacttaacctacttctctccaaggaaacagaaggttacgatgatgttcacttgattggtaatagcttgacagatttggaaagcagtgtggcaagtaaacttctccagtatgttcatcgcactcagatgcgtgagctcagtcatttacaaaaagctcagcactatgagataaaggattacttgcagatgtcctatgcgaccaagtcaagcttggatttactggaaaatgctagaacgggcaagaaacacggctctcttttctggctcttggataaaaccaagacagccatgggaatgcgtctcttgcgaacttggattgatcgtcctttagtgaatcaagccagcatcatagagcgtcagaatattatccaagttttcttggacaatttctttgagcgtagtgacttgaccgagagtctaaaaggtgtttacgatattgagcgtctggccagtcgtgtttcctttggcaaggctaatcctaaagatttaattcagctaggtcataccttagctcaggttccggtcattaaggcaatcttggagtcctttaatgacgataccttgtcaggtcttttacaagagttagatgctttgccagaattagagagtttgattcgttcggctattgatcctgatgctccagcaacaattacagaaggtggtattattcgtgatggttttgatgagaccttggacaagtatcgcaaagtgatgagcgaggggacgtcatggattgcggatatcgaagcaaaagaacgcgaagcgtctggtattacaacactaaaaattgattataaccgtaaagatggttattacttccatgtgaccaactctaaccttagtttggttcctgaccatttcttccgtaaggcgacactgaaaaattcggagcgttttggaactgctgagttggctaagattgaaggtgaaatgctagaagcacgtgaaaaatcgtcaaccctagagtatgatattttcatgcgtgtccgtgagcaggttgaacgctatatcgaccgtttgcagtctttggctaaggccattgcaacagtcgatgtgcttcagagtttggctgttacagcggagacaaatcattatgttcgtcctgtttttaatgatgagcatcgtatcgctattgatcggggccgtcatgcggtggttgagaaggttatgggcgttcaggaatacattccaaatacgattacctttgacagtcagaccaatattcagctcatcacaggaccaaatatgagtggtaaatctacctatatgcggcagttagccttgtcagtagttatggctcaaatgggggcttacgtaccagcagatagtgttgatttacctgtttttgacgccatctatacacgtataggtgccgcagatgacctgatttcaggtcagtcgaccttcatggttgagatgatggaggctaatcaggccattaagcgagcgacacctaattccttgattatttttgatgagttggggcgtgggacagccacttatgatggtatggctttggcccagtctattattgagtttattcatgacaaggttggggctaagaccatgtttgctacccattatcatgagttgactgcgctgtcaaacagtttgatacaccttgtcaacgtccatgttgcgacacttgaaaaggatggcgaagtgactttcctccacaaaattgtcgatggtccagccgataaatcgtatggtattcatgtagctaaaattgcaggtttaccaacagatttattgaaccgtgctgacaccatattgacacaattagaaggagagacagttgtaatccaacctcaggaaaaggtatcgtctcaagaaaagcctgccattgaaacgcatgtcaacgagcaaatctctctctttgatgactttactgaaaatccggttcttcaagagttgcgtgatttggacatttacaacatgacaccgatgcaagtcatgatggctgtagcagatttaaaacagaaactataa

SEQ ID NO: 154 - protein, 852 aa, synthetic construct
MAKEKISPGMQQYLDIKKNYPDAFLLFRMGDFYELFYDDAVKAAQILEISLTSRNKNADNPIPMAGVPYHSAQAYIDVLVEMGYKVAIAEQMEDPKQAVGVVKREVVQVITPGTVVDSSKPDSANNFLVAIDKVGSRFGLSYMDVSTGEFFATELDDFSSVCSEIQNLKAREVVVGYDLLETDEQVLVNQLNLLLSKETEGYDDVHLIGNSLTDLESSVASKLLQYVHRTQMRELSHLQKAQHYEIKDYLQMSYATKSSLDLLENARTGKKHGSLFWLLDKTKTAMGMRLLRTWIDRPLVNQASIIERQNIIQVFLDNFFERSDLTESLKGVYDIERLASRVSFGKANPKDLIQLGHTLAQVPVIKAILESFNDDTLSGLLQELDALPELESLIRSAIDPDAPATITEGGIIRDGFDETLDKYRKVMSEGTSWIADIEAKEREASGITTLKIDYNRKDGYYFHVTNSNLSLVPDHFFRKATLKNSERFGTAELAKIEGEMLEAREKSSTLEYDIFMRVREQVERYIDRLQSLAKAIATVDVLQSLAVTAETNHYVRPVFNDEHRIAIDRGRHAVVEKVMGVQEYIPNTITFDSQTNIQLITGPNMSGKSTYMRQLALSVVMAQMGAYVPADSVDLPVFDAIYTRIGAADDLISGQSTFMVEMMEANQAIKRATPNSLIIFDELGRGTATYDGMALAQSIIEFIHDKVGAKTMFATHYHELTALSNSLIHLVNVHVATLEKDGEVTFLHKIVDGPADKSYGIHVAKIAGLPTDLLNRADTILTQLEGETVVIQPQEKVSSQEKPAIETHVNEQISLFDDFTENPVLQELRDLDIYNMTPMQVMMAVADLKQKL

SEQ ID NO: 155 - DNA, 831 bp, synthetic construct
  source 1..831: mol_type = other DNA
atgaggagcaaggttaaataccaagcagctcaccaagagttggtagcttcatctatggcgactaagttggctaacgagattgatcctgaaaataaggttggttgtatgttggcagccggtcagtattatccaaatacagcccacccacatgactactgggcagccatgcaagaagatcgtagtaactatttcttcattgatgtccagaatcgtggagagtatccaaattatgctaaaaagcagtgggagcgtgatggtattaagcttgagatgactgaagaagatttggagcttctcaaagagcatactgttgatttcattttcttttcttattattcaagtcgtgtggcttcaggtgatccagaagttaatgaaaagactgccggcaatatctttgcccctattaaaaatccatatttagaatcttcagagtggggttggcaaattgacccactaggccttcgtattaccctcaatgctatttgggatcgttaccagaaaccgatgtttatcgttgagaatgggattggtgctgtggatacaccagatgaaaatggttatgttgaagacgattatcgtattgactacttggcagcccacattaataccatgagggatgccatcaatgaagatggtgtagaactttggggatacacaacctggggttgtatcgatcttgtttctgccggaactggtgaaatgaaaaaacattatggtttcatctatatagaccgcgacaatgatggtaagggtagccttaaaagctctaagaaaaaatcatttaacaggtacaaagaagttatcgcaagtaacggtgcctctgtagaatag

SEQ ID NO: 156 - protein, 276 aa, synthetic construct
MRSKVKYQAAHQELVASSMATKLANEIDPENKVGCMLAAGQYYPNTAHPHDYWAAMQEDRSNYFFIDVQNRGEYPNYAKKQWERDGIKLEMTEEDLELLKEHTVDFIFFSYYSSRVASGDPEVNEKTAGNIFAPIKNPYLESSEWGWQIDPLGLRITLNAIWDRYQKPMFIVENGIGAVDTPDENGYVEDDYRIDYLAAHINTMRDAINEDGVELWGYTTWGCIDLVSAGTGEMKKHYGFIYIDRDNDGKGSLKSSKKKSFNRYKEVIASNGASVE

SEQ ID NO: 157 - DNA, 675 bp, Streptococcus thermophilus
  source 1..675: mol_type = genomic DNA
atgtcaaatctcattttcgactcagtcacaaaaatttttggtgaggggggtagtaagtacgtagcactagaaaatattaatttcgaagcagaaagcggtcagcttattttagttgtagggccatcaggatcaggaaaaactacatttttgactatagcaggcggactacagacaccaactaatggtgacgtaaaaataaatgacagtacaattaacagtttgtccaaaaaacaacaaacaaaattaagattggaaaaaataggatttattttacaatcttataacttagtcccatttttaacggtcgaagaacagtttaaattcgtagataaactaaaaaaacaaaatttgactgaacaaaaattgcatgagcttctttctgatttaggacttttagaactgctaaaaaaatatcctaatcaactttctggtggacaaaagcagcgtgtggcaattgctagagcattatacactgatcctgactatattttagcagatgaacccacagccacattagatacagatagatccatgaaagtaatcgagttactcagagatttagcgcacaaaagaaataaaattattattgtagttactcatgacttaaggttaaaagatatggcagacaaagtttatcagataattgacggtaaaatgacaatcctggagaagtaa

SEQ ID NO: 158 - protein, 224 aa, Streptococcus thermophilus
MSNLIFDSVTKIFGEGGSKYVALENINFEAESGQLILVVGPSGSGKTTFLTIAGGLQTPTNGDVKINDSTINSLSKKQQTKLRLEKIGFILQSYNLVPFLTVEEQFKFVDKLKKQNLTEQKLHELLSDLGLLELLKKYPNQLSGGQKQRVAIARALYTDPDYILADEPTATLDTDRSMKVIELLRDLAHKRNKIIIVVTHDLRLKDMADKVYQIIDGKMTILEK

SEQ ID NO: 159 - DNA, 720 bp, Streptococcus thermophilus
  source 1..720: mol_type = genomic DNA
atgtctgaagccataaaattatttcaatataatactttgggagccttgatggctgggctttatggaggtactttaaccgttggagagttgttagagcatggagacttgggtctcggaacattagattctatcgatggtgagttgattgttcttgacggtaaagcctaccaagctaaaggctctgaaggtaaagtcgaagtggtagaagtttctcctgatgaaaaagttccctatgcagcagtcgtacctcaccaagctgaagttatcttccgtcaacgttatgaaatgactgataaagaacttgaggatcgtattgaatcatactatgatggggtcaatcttttccgttcaatcaaaatcaaaggtcattttaaacacatgcacgtgcgcatgattccaaaatcaaatgcggacattaagtttgctgatgttgccactcgccaacctgagtatgaggtagatgatatttcagatacaatcgttggtatctggacacctgaaatgttccacggtgtgagtgttgctggttaccatctccattttatttcagatgacttgacattcggtggtcacgttatagactttgttatcgagaatggtattattgaagttggaccagttgaccaactcgatcaacgcttcccagttcaagaccgtcaatatctctttgctaaattcaatgttgatgaaatgcgtaaagatattactaaggctgaataa

SEQ ID NO: 160 - protein, 239 aa, Streptococcus thermophilus
MSEAIKLFQYNTLGALMAGLYGGTLTVGELLEHGDLGLGTLDSIDGELIVLDGKAYQAKGSEGKVEVVEVSPDEKVPYAAVVPHQAEVIFRQRYEMTDKELEDRIESYYDGVNLFRSIKIKGHFKHMHVRMIPKSNADIKFADVATRQPEYEVDDISDTIVGIWTPEMFHGVSVAGYHLHFISDDLTFGGHVIDFVIENGIIEVGPVDQLDQRFPVQDRQYLFAKFNVDEMRKDITKAE

SEQ ID NO: 161 - DNA, 3726 bp, Streptococcus thermophilus
  source 1..3726: mol_type = genomic DNA
atggataaacgtattttcgttgagaaaaaagctgattttcgtgtgaaatctcgctctttattaaaagaattacagcataatattcagttgaaaaccttgaatgatcttcgtattgtacaagtttacgatgtctttaatttggcagaggatttgtttgcccgtgcggaaaaatatatcttttctgaacaagtgaccgatactgttttggatgaagctacagttaagactgaccttgagaagtatgctttctttgctatcgaaagcttgcctggtcaatttgatcaacgtgcagcatcttcacaagaagctttgcttttgcttggtagctcaagtgatgtaacggttaatactgctcaattgtacttggttaacaaggacattgctgctaatgaattggaagctgttaaaaattatctattgaacccagtggactctcgtttcaaagatatcactgttggtattgctaaacaggatttctctgagtctgacaaaacgattccaaacttggatttctttgaaacttatacagcagaagattttgcacagtataaggcagatcaaggattggccatggaagtggatgacctgctcttcattcaagattacttcaagtcaattggacgtgtgccaactgagactgaacttaaggttttggatacttactggtctgaccactgccgccacacaactttcgagactgagttgaaaaacatcgacttctcagcttctaaattccaaaaacaattgcaagcaacttatgacaaatatattgccatgcgtgatgagttgggacgtgcagaaaagcctcaaaccttaatggatatggcgactatttttggtcgttatgagcgtgccaatggccgtttggatgacatggaagtttctgacgaaatcaatgcctgctcagttgaaattgaagtagatgtcaatggtgtcaaagaaccatggcttcttatgttcaagaacgaaactcataaccacccaacgaaaatcgaaccatttggtggagcggctacttgtatcggtggtgccattcgtgacccattgtcaggtcgttcatacgtttaccaagctatgcgtatttcaggtgccggtgatattacaacaccaatcgctgaaacacgtgctggaaaattgccacaacaagtcatttctaaaaaagcggctcatggttattcttcatatggtaaccaaattggtcttgcaacaacttatgttcgtgaatacttccacccaggtttcgttgccaagcgtatggagctaggtgcagttgtcggtgcagctcctaaggagaatgttgtccgtgaaaaacctgaagctggagatgtggttatcttgctcggtggtaagactggacgcgacggtatcggtggtgcgacaggttcttcaaaagttcaaacggttgaatcagttgaaacagctggtgctgaggttcaaaaaggtaatgctatcgaagaacgtaagattcaacgtcttttccgcaatggtgaagtaacacgtcttatcaagaaatccaatgactttggtgctggtggtgtctgtgtggctatcggtgaattggcagatggtcttgaaattgatctagacaaagtgccattgaaataccaaggtttgaacggtaccgaaattgccatctctgaatctcaagaacgtatggctgtggtggttcgtccagaagatgtagatatcttcgttgctgaatgtaacaaagaaaatattgatgcagttgtcgttgcaaccgtgactgaaaaaccaaatctcgtcatgcactggaatggtaaaacaattgttgatttggaacgtcgtttccttgatacaaatggtgtgcgtacagtagtagatgttaaggtggttgacaaggatgtcaagcttccagaagaacgcaaaacctctgctgaaacccttgaagcagatacgcttgaagttttggctgaccttaaccatgcaagtcaaaaaggtttacaaactatgtttgatagctcggttggtcgttcaacagtcaatcacccacttggtggtcgataccaaattactccaactgaagcttctgttcagaaattgccagtccaatatggcgtgacaacaactgcctctgtcatagcccaaggatttaacccatacatcgcagaatggtctccatatcatggtgctgcatatgcagtcatcgaagcaacagctcgtttggttgctgttggtgcaaattggtctaaggctcgtttctcttaccaagaatacttcgagcgtatggataaaaaagcagagcgttttggtcaaccagtatcagctcttcttggatcaatcgaagctcagattcaacttggtttgccatctattggtggtaaggactctatgtcaggcacctttgaagaattgacagtaccaccaaccttggttgcttttggggtgacaacagcagacagtcgtaaggtcctttctccagaatttaaggctgctggtgaaaatatctactacatcccaggtcaagctttggcccaagagattgattttgatctcatcaagtctaactttgctcagtttgaagctatccaagctgatcacaaagtgacagctgcatcagctgtcaaatatggtggtgtcattgaagctcttgcacttgcaacgtttgggaaccatatcggggcgactgtaacccttgaaaatcttgagactgccttaacagctcaattgggaggattcatcttcacatctccggaagagattgcaagtgttggcaagattggacaaacagtagctgactttacacttgctgtcaacggtgtaacgcttgatggatataaacttgacagtgcctttcaaggtaaattggaagaggtttacccaactgaatttgcacaagcaactgagttagaagaagtacctgctgtgacatcagatactgttataaaagctaaagaagtagttgagaaaccagtggtttacatcccagtattcccaggtactaactctgagtatgactctgctaaggcctttgaaaaagaaggtgccaaagtcaacttagtaccatttgtaacattgaatgaagaagcgattgtcaagtctgttgacaccatggttgacaatattgaaaaagctaacattatcttccttgcaggtggcttctcagcagcggatgaaccagatggagcagctaaatttatcgttaacatcttgctcaatgaaaaagtacgtgcagctattgatagcttcatcgaacgtggtggtttaatcatcggtatctgtaacgggttccaagctcttgtcaaatcaggtctccttccatacggtaactttgaagatgcaagcagtactagtccaaccctcttctacaatgatgccaaccaacacgtggctaagatggttgaaacacgtattgccaatactaactcaccttggcttgctggagtgaaagttggtgacatccataccatcccagtatcacacggtgaaggtaaatttgtcgtgacgactgaagaatttgcggagcttcgcgataatggtcaaatctttagccaatatgttgacttcgaaggcaaacctagcatggattctaaatataatccaaatggttctgtcaatgctattgaaggtatcacaagcaagaacggtcaaattattggtaagatgggacactcagaacgtttcgaagatggtctcttcaaaaatattccaggaaataaagaacagcacctatttgcgtcagcggttaaatacttcactggaaaataa

SEQ ID NO: 162 - protein, 1241 aa, Streptococcus thermophilus
MDKRIFVEKKADFRVKSRSLLKELQHNIQLKTLNDLRIVQVYDVFNLAEDLFARAEKYIFSEQVTDTVLDEATVKTDLEKYAFFAIESLPGQFDQRAASSQEALLLLGSSSDVTVNTAQLYLVNKDIAANELEAVKNYLLNPVDSRFKDITVGIAKQDFSESDKTIPNLDFFETYTAEDFAQYKADQGLAMEVDDLLFIQDYFKSIGRVPTETELKVLDTYWSDHCRHTTFETELKNIDFSASKFQKQLQATYDKYIAMRDELGRAEKPQTLMDMATIFGRYERANGRLDDMEVSDEINACSVEIEVDVNGVKEPWLLMFKNETHNHPTKIEPFGGAATCIGGAIRDPLSGRSYVYQAMRISGAGDITTPIAETRAGKLPQQVISKKAAHGYSSYGNQIGLATTYVREYFHPGFVAKRMELGAVVGAAPKENVVREKPEAGDVVILLGGKTGRDGIGGATGSSKVQTVESVETAGAEVQKGNAIEERKIQRLFRNGEVTRLIKKSNDFGAGGVCVAIGELADGLEIDLDKVPLKYQGLNGTEIAISESQERMAVVVRPEDVDIFVAECNKENIDAVVVATVTEKPNLVMHWNGKTIVDLERRFLDTNGVRTVVDVKVVDKDVKLPEERKTSAETLEADTLEVLADLNHASQKGLQTMFDSSVGRSTVNHPLGGRYQITPTEASVQKLPVQYGVTTTASVIAQGFNPYIAEWSPYHGAAYAVIEATARLVAVGANWSKARFSYQEYFERMDKKAERFGQPVSALLGSIEAQIQLGLPSIGGKDSMSGTFEELTVPPTLVAFGVTTADSRKVLSPEFKAAGENIYYIPGQALAQEIDFDLIKSNFAQFEAIQADHKVTAASAVKYGGVIEALALATFGNHIGATVTLENLETALTAQLGGFIFTSPEEIASVGKIGQTVADFTLAVNGVTLDGYKLDSAFQGKLEEVYPTEFAQATELEEVPAVTSDTVIKAKEVVEKPVVYIPVFPGTNSEYDSAKAFEKEGAKVNLVPFVTLNEEAIVKSVDTMVDNIEKANIIFLAGGFSAADEPDGAAKFIVNILLNEKVRAAIDSFIERGGLIIGICNGFQALVKSGLLPYGNFEDASSTSPTLFYNDANQHVAKMVETRIANTNSPWLAGVKVGDIHTIPVSHGEGKFVVTTEEFAELRDNGQIFSQYVDFEGKPSMDSKYNPNGSVNAIEGITSKNGQIIGKMGHSERFEDGLFKNIPGNKEQHLFASAVKYFTGK

SEQ ID NO: 163 - DNA, 768 bp, Streptococcus thermophilus
  source 1..768: mol_type = genomic DNA
atggcgacaattaaagaagtcaaggaacaattggctattttgagggatcttgacgaccctagatgggcaagttttgaggaagatagtcggactggtgtacaagctgccattaggaaacgccgtaaggctatcctcgctgaacttgctgaggaagaacgtttagaaacgcttcttaattatgagaaatcactctatgctcgaggaatagaactcatcgcaggtgtcgatgaagttggacgtggtcctttagctgggccagttgtagcggctgcggtcattttaccaaagctttgcaaaataaaaggtctaaatgacagtaaaaagattccaaaatctaaacatgaagctatctataatcaggtaatgaaggaggcagtagctgtcggtattggtattaaggataattatgtgattgatgacgtcaatatttatgaagctaccaagttagccatgatagaagctattgaaaaactcaatccccaacctgaacatttgctaattgatgccatgaatttggacttacctatagaacaaacctcaatcataaaaggggatgctaattctctatctattgcagcagcttctatcgcagcaaaagtaactcaggataagatgatggctgattatgagcaggagtttcctggatacgcctttgccaaaaatgcaggctatggcaccaaagaacatttgtctggcatagataagtttggtgtcacgccaattcatagaagaagcttcgaaccaataaagtcaataataaaaaagtag

SEQ ID NO: 164 - protein, 255 aa, Streptococcus thermophilus
MATIKEVKEQLAILRDLDDPRWASFEEDSRTGVQAAIRKRRKAILAELAEEERLETLLNYEKSLYARGIELIAGVDEVGRGPLAGPVVAAAVILPKLCKIKGLNDSKKIPKSKHEAIYNQVMKEAVAVGIGIKDNYVIDDVNIYEATKLAMIEAIEKLNPQPEHLLIDAMNLDLPIEQTSIIKGDANSLSIAAASIAAKVTQDKMMADYEQEFPGYAFAKNAGYGTKEHLSGIDKFGVTPIHRRSFEPIKSIIKK

SEQ ID NO: 165 - DNA, 3111 bp, Streptococcus thermophilus
  source 1..3111: mol_type = genomic DNA
atgtttgcacaattggatacaaaatcagtatatagcttcatggacagtgttgtagatttaaacacctatgtaaaacgtgccaaagagcttggttatcaaagcattggtttgatggatcgtgataatctctatgcggcctatcattttgcccagcttgcaatcagaaatggtttacgtcctttaattggaacagaagctaatttattctatgaagggagaaaaattccctttcgcctattagctaaaaataatcagggctataaaaacttgatgaagttggcaacagagctgtcttcaggacagcgggagtttacatatttttcagactatctgaatgacattgccctaattcttccaagtgaggactgggagcctgggatgagtcttggctcagaagttttcattggggtaagacctgaggatgctggtaaagagtttgattatcctgtggttcctttacatacagtgcgttactttgaaaatgcagatcgctcgactatccagacgttacatgctatctcacaaaatgtgtccttgtctgaggtatccatctgtcctatgaatcagctccttttctcggccaaagaaatggaagaagcttatagtaagctttcagaggctttaaacaatttgaatcagcttgtttcagacgtctcttatcaatttgatacaaatctcaagcttcctcgttttaatcgtgaaatgccagcagttgatcaattgcgtcagttagctcaatctggattagatagtaaagcgttaagagaaccagcttatcaggaacgtttagacaaggaattgtcaataatacatcaaatgggttttgatgactatttcttgattgtttgggatttgttacgttttggtcgtagtcgaggttattatatgggaatgggacgtgggtctgctgcagggagtttggtagcttatgtccttgatattactggaattgatccagttaaaaatgatttacttttcgagcgtttcttaaatgaggaacgttatagtatgcctgatatcgatatagacttacctgatatttatcgaagtgagtttcttcattacgttagagaccgttatggtagtaagcactctgcacagattgttactttttcaacttttggggctaagcaggctatccgagatgtttttaaacgttttggagctaaggaatttgaattatcccaactcagtaaaaaaatatcttttcgagatactttgacaactgtctatgagaaaaatatgtcattcagacagctaattaatcagaggcaggaatttcagagagcttttgaaattgctaaggctattgaaggccagccaagacaaacctccatacatgctgcaggtatcgtgatgagcgatgatgacctgaccaatcatattcctcttaaggcaggtgaggacatgatggttacacagtacgatgcttccgcagtcgaggccaatggtttgcttaagatggatttcttggggcttcgaaatttgacctttgttcaaaaaatgaaggagaagcttgaagaagaacagggaatcgctatcgatatcaaatccattgacttggaagattcacaaaccctagcgctttttgccagtggtaagacaaagggaatttttcaattcgagcaggcaggtgctataaatctccttaaacggattaaacctgctaagttcgaagaagtagtagcgacaacctctctcaaccgtccaggagctagcgattatacagataatttcgttgctcgtaaatatggtaaggagaaagtagatctcattgacccagtggttgctccaattcttgaacctacctatggtatcatgctttatcaggagcaggttatgcagattgcccaagtatttgctggctttagccttggaaaggccgatttgcttcgtcgggcaatgtctaaaaaagatgccaatgaaatgtccaaaatgtccgaacaattcatggaaggaagtcgtcagttagggcgtgacccacaggttgctaaacgtctctttagtatgataactaagtttgcaggatatggttttaataaaagtcatgcctatgcttattcatccctagccttccaattagcttattttaaaacgcattatcctcagatattctatgacgtcatgttaaattattcgtcaagtgattacattagggatgcaattgaaaatggcttttccttagctagcctggatattaatagaattccttatatggacaagatttccgatggaaagattgtactaggtctaaaaaccataaaaggtttgcccagagattttgcactttggattattgaggatcgccaacaaaatggtaaatacatttctatagaggattttttgacacgtattcctagtaaatatcaaaaagtagatattttgacacctttgatccaaataggattatttgatcgttttgagcctaatagacggaaaatcatcggaaacttaagcaacctctttacttttgttaatgaattagggagtttgtttgcagagtcgtcttattcttgggtggagtatgaagactatagtcagacagaacgctacagtatagagcaggatttactaggagttggacttagtacacatccattacatttagctcaaaaaatggcatcacggccctaccaaccaatttctgagttgactttaaacagtaaggctacagttttggtccagcttgaatcagttcgcattatcagaacgaaaaagggagaccagatggctttcctaagtgtttttgatggggtgaataaactggatgtcactctttttccagaaacttatttctatcacaaagataagttaagcgaaggtagtctcttttatctagaaggacgaactcaagagcgcgacggacgtcttcaattaattttgtcaaatatagaggaggctagcactgagcgtttctggattttactagagaatcataataaagatttagaagtttctcacattttatccaagtatcctggacatattcctgtggttattcgttatcagggaagcaaggaaacaatagttagtcaacgatataaagtgtctaagaacgaagaactcagtagggaattagcaccttttgtgttgaaaacggttttacgttaa

SEQ ID NO: 166 - protein, 1036 aa, Streptococcus thermophilus
MFAQLDTKSVYSFMDSVVDLNTYVKRAKELGYQSIGLMDRDNLYAAYHFAQLAIRNGLRPLIGTEANLFYEGRKIPFRLLAKNNQGYKNLMKLATELSSGQREFTYFSDYLNDIALILPSEDWEPGMSLGSEVFIGVRPEDAGKEFDYPVVPLHTVRYFENADRSTIQTLHAISQNVSLSEVSICPMNQLLFSAKEMEEAYSKLSEALNNLNQLVSDVSYQFDTNLKLPRFNREMPAVDQLRQLAQSGLDSKALREPAYQERLDKELSIIHQMGFDDYFLIVWDLLRFGRSRGYYMGMGRGSAAGSLVAYVLDITGIDPVKNDLLFERFLNEERYSMPDIDIDLPDIYRSEFLHYVRDRYGSKHSAQIVTFSTFGAKQAIRDVFKRFGAKEFELSQLSKKISFRDTLTTVYEKNMSFRQLINQRQEFQRAFEIAKAIEGQPRQTSIHAAGIVMSDDDLTNHIPLKAGEDMMVTQYDASAVEANGLLKMDFLGLRNLTFVQKMKEKLEEEQGIAIDIKSIDLEDSQTLALFASGKTKGIFQFEQAGAINLLKRIKPAKFEEVVATTSLNRPGASDYTDNFVARKYGKEKVDLIDPVVAPILEPTYGIMLYQEQVMQIAQVFAGFSLGKADLLRRAMSKKDANEMSKMSEQFMEGSRQLGRDPQVAKRLFSMITKFAGYGFNKSHAYAYSSLAFQLAYFKTHYPQIFYDVMLNYSSSDYIRDAIENGFSLASLDINRIPYMDKISDGKIVLGLKTIKGLPRDFALWIIEDRQQNGKYISIEDFLTRIPSKYQKVDILTPLIQIGLFDRFEPNRRKIIGNLSNLFTFVNELGSLFAESSYSWVEYEDYSQTERYSIEQDLLGVGLSTHPLHLAQKMASRPYQPISELTLNSKATVLVQLESVRIIRTKKGDQMAFLSVFDGVNKLDVTLFPETYFYHKDKLSEGSLFYLEGRTQERDGRLQLILSNIEEASTERFWILLENHNKDLEVSHILSKYPGHIPVVIRYQGSKETIVSQRYKVSKNEELSRELAPFVLKTVLR

SEQ ID NO: 167 - DNA, 1737 bp, Streptococcus thermophilus
  source 1..1737: mol_type = genomic DNA
atgcctaagctcggtgttgatatgcaggaaggtgaaatcattgagtggaaaaaacaagagggagacgtggttaatgagggagatatccttcttgaaatcatgtcagataagacaaacatggaacttgaagcagaagactcaggtgttcttttgaagattacccgtcaagctggtgaaacagtaccagttactgaggtgattgattatatcggagctgaaggagaagttgtggctgataattctacaagcgcaccagctgcagaagcaactgctcaattggaagcggctgggcttgaagtccctaaagctccatctcaaccatctccagcgacagcagaaaaagcagcccttgctgataacgaatatgatatcgttgttgttggtggtggtccagctggttactatgcagccatccgtggagcacaacttggcggtaaggttgccatcgttgaaaaatcagaatttggtggaacatgtttgaataagggatgtatcccaactaagacctaccttaaaaacgcagaaatccttgatggacttaaaattgcagcaggtcgtggtatcaacctagcatcaacaaactacaccatcgatatggataagacggttgactttaaaaactctgttgttaagacccttactggtggtgtgcaaggtcttttgaaagctaataaggtaactattttcaatggtcttggtcaagttaaccctgataagactgtcactattggctcagaaacaatcaagggacgtaacatcatccttgcaactggatcaaaagtatcacgtattgatatccctggaatcgattcacaacttgtgatgacatcagatgatattcttgatttgcgtgaattgcctaaatcacttgctgttatgggtggtggtgtcgttggtatcgagcttggtcttgtttatgcttcatacggcgttgacgtaacagttgtagagatggctgatcgtatcatacctgctatggataaagaagtgtcacttgagcttcaaaaaatcctttctaagaagggtatgaagattatgacatctgttggtgtttctgaaattgttgaaggcaataaccaattaacacttaaactcaataatggcgaagagattgtggctgaacgtgcccttctatcaatcgggcgtgttccacaattgaacggtcttgaaaatcttaaccttgagctcgaccgtggccgtatcaaagtaaatgaataccaagaaacttctatccctggtatttacgcccctggtgatgtaaacggaactaagatgcttgctcacgctgcttaccgtatgggtgaagtagcagccgaaaatgctatgcatggtaatgtccgtaaggcacaccttgattatacaccagcggcagtttacacccacccagaggtagcaatgtgtggtctaactgaagaagatgcacgcgctaaatatggtgatgttctcattggtaaatcaagctttgctgggaatggacgtgctatcgcctcaaatgaagctcatggctttgttaaagtcgttgcggatgcgaaataccatgaaatccttggagttcacattattggtccggctgctgctgagttgattaacgaagcagcaactattatggaaaatgaactgactgtagatgaactcttgcaatctatccatggtcacccaactttctctgaaaatatgtatgaggcttttgctgatgtacttggagaagctgttcataacccaccaaaacgtaaataa

SEQ ID NO: 168 - protein, 578 aa, Streptococcus thermophilus
MPKLGVDMQEGEIIEWKKQEGDVVNEGDILLEIMSDKTNMELEAEDSGVLLKITRQAGETVPVTEVIDYIGAEGEVVADNSTSAPAAEATAQLEAAGLEVPKAPSQPSPATAEKAALADNEYDIVVVGGGPAGYYAAIRGAQLGGKVAIVEKSEFGGTCLNKGCIPTKTYLKNAEILDGLKIAAGRGINLASTNYTIDMDKTVDFKNSVVKTLTGGVQGLLKANKVTIFNGLGQVNPDKTVTIGSETIKGRNIILATGSKVSRIDIPGIDSQLVMTSDDILDLRELPKSLAVMGGGVVGIELGLVYASYGVDVTVVEMADRIIPAMDKEVSLELQKILSKKGMKIMTSVGVSEIVEGNNQLTLKLNNGEEIVAERALLSIGRVPQLNGLENLNLELDRGRIKVNEYQETSIPGIYAPGDVNGTKMLAHAAYRMGEVAAENAMHGNVRKAHLDYTPAAVYTHPEVAMCGLTEEDARAKYGDVLIGKSSFAGNGRAIASNEAHGFVKVVADAKYHEILGVHIIGPAAAELINEAATIMENELTVDELLQSIHGHPTFSENMYEAFADVLGEAVHNPPKRK

SEQ ID NO: 169 - DNA, 675 bp, Streptococcus thermophilus
  source 1..675: mol_type = genomic DNA
atgtcaaatctaattttcgactcagtcacaaaaatttttggtaaagggagtagtaagtacgtagcactagaaaatattaatttcgaagcagaaagcggtcagcttattttagttgtagggccatcaggatcagggaaaactacatttttgactatagcaggcggactacagacaccaactaatggtgacgtaaaaataaatgacagtacaattaacagtttgtccaaaaaacaacaaacaaaattaagattggaaaaaataggatttattttacaatcttataacttagtcccatttttaacggtcgaagaatagtttaaattcgtagataaactaaaaaaacaaaatttgactgaacaaaaattgcatgagcttctttctgatttaggacttttagaactgctaaaaaaatatcctaatcaactttctggtggacaaaagcagcgtgtggcaattgctagagcattatacactgatcctgactatattttagcagatgaacccacagccgcattagatacagatagatccatgaaagtaatcgagttactcagagatttagcgcacaaaagaaataaaattattattgtagttactcatgacttaaggttaaaagatatggcagacaaagtttatcagataattgacggtaaaatgacaatcctggagaagtaa

SEQ ID NO: 170 - protein, 223 aa, Streptococcus thermophilus
MSNLIFDSVTKIFGKGSSKYVALENINFEAESGQLILVVGPSGSGKTTFLTIAGGLQTPTNGDVKINDSTINSLSKKQQTKLRLEKIGFILQSYNLVPFLTVEEFKFVDKLKKQNLTEQKLHELLSDLGLLELLKKYPNQLSGGQKQRVAIARALYTDPDYILADEPTAALDTDRSMKVIELLRDLAHKRNKIIIVVTHDLRLKDMADKVYQIIDGKMTILEK

SEQ ID NO: 171 - DNA, 804 bp, Streptococcus thermophilus
  source 1..804: mol_type = genomic DNA
atgacattttgcagtatagaaagggttgtcatgattttaattgaagatttaaccgttgtgagtcagcgtgagattgcacctcgtatatttgagatggtgcttaagggggagatggttgcggatatgcagcttggtcagtttgtccacctcaaagtgccagacccaagcaagctccttcgtcgtccgatttccatctcagagattgactacaataaaaaagaggcaaccattgtctatcgtgtggagcgtgagggaacagctatcctttcaaagatggtagccggtcaaactattgataccatgggccctcaaggaaatggttttgatatttccattattgaggctgggcagaaagctctccttgttggtggtggtattggcgtgccacctttggttgagacggctaagcagttgaaggctaagggagtggaagttgtatcagtaattggtttcgccaataagaatgccgttattttggaggataaactcagggcttgtggtgatgtttatgtgacgactgacgacggttcatacggtatcaagggttacgtttcgacagtcattgataactttgactggacaccagatgccgtttacagttgtggagctccaggtatgctcaaatatgttgatagcaagtttgaaaatcatcctcacgcctatgtttctatggaagcccgtatggcttgtggaatggaggcgtgctatgcctgtgtagttcacgttaaaggagaaacggatgctaaaaatcttcgtgtttgcgaggagggaccagtctttcctaccggaaaagtcatcgtctag

SEQ ID NO: 172 - protein, 267 aa, Streptococcus thermophilus
MTFCSIERVVMILIEDLTVVSQREIAPRIFEMVLKGEMVADMQLGQFVHLKVPDPSKLLRRPISISEIDYNKKEATIVYRVEREGTAILSKMVAGQTIDTMGPQGNGFDISIIEAGQKALLVGGGIGVPPLVETAKQLKAKGVEVVSVIGFANKNAVILEDKLRACGDVYVTTDDGSYGIKGYVSTVIDNFDWTPDAVYSCGAPGMLKYVDSKFENHPHAYVSMEARMACGMEACYACVVHVKGETDAKNLRVCEEGPVFPTGKVIV

SEQ ID NO: 173 - DNA, 1086 bp, Streptococcus thermophilus
  source 1..1086: mol_type = genomic DNA
atgacagaaaataaaaatgtaatattatcggctcgcgatatcgtcgtggaatttgacgttcgtgaccgtgttttgacagctattcgaggggtttctcttgatttagttgaaggtgaagttttagccttggttggtgagtctggttcaggaaaatcagtcttgactaaaacttttacagggatgctggaagaaaatggtcgtgtagctagcgaatctatcgactaccgtggcaaagacttgactaaatttaagagtcaccaagattgggcagctatccgtggagctaagattgcaactattttccaagatccaatgaccagtcttaacccaattaagactatcggaagtcaaattattgaagttattgttaagcaccaagggaaaacagccaaggaagctaaaaaaatggcaattgattacatggacaaggttggtattccagatgctgaaaaacgtttcaatgaatatcctttccaatactctggtgggatgcgtcaacgtatcgttattgccattgccttggcttgtcgtcctgatgtccttatctgtgacgaaccaacaactgcccttgatgtgaccattcaagctcaaatcattgacctcttgaaatcactcaaagaggagtatggtttctcggtcattttcattacccatgaccttggcgtcgtagcaagtatcgcggacaaggttgctgtcatgtatgctggcgaaatcattgaatacgcaactgttgaggaaattttctatgagccttgtcatccatacacgtggagcttgctttcaagtctgccacaattggctgatgataatggaaaactcttctcaattccagggacgccaccatcactttatacaccagttgttggggatgcctttgccttgcgttcagattatgctttgcagattgattttgaagaaaaagcaccacaattccaagtttcagatactcactgggctaagacctggctcttacatgaggatgcgccaaaagttgataaacctgcggttatccaaaatctacatgaaaaaatcctagccaatatgggatttgcacatttaggagatgaggaggaaggcaatgcctga

SEQ ID NO: 174 - protein, 361 aa, Streptococcus thermophilus
MTENKNVILSARDIVVEFDVRDRVLTAIRGVSLDLVEGEVLALVGESGSGKSVLTKTFTGMLEENGRVASESIDYRGKDLTKFKSHQDWAAIRGAKIATIFQDPMTSLNPIKTIGSQIIEVIVKHQGKTAKEAKKMAIDYMDKVGIPDAEKRFNEYPFQYSGGMRQRIVIAIALACRPDVLICDEPTTALDVTIQAQIIDLLKSLKEEYGFSVIFITHDLGVVASIADKVAVMYAGEIIEYATVEEIFYEPCHPYTWSLLSSLPQLADDNGKLFSIPGTPPSLYTPVVGDAFALRSDYALQIDFEEKAPQFQVSDTHWAKTWLLHEDAPKVDKPAVIQNLHEKILANMGFAHLGDEEEGNA

SEQ ID NO: 175 - DNA, 1131 bp, Streptococcus thermophilus
  source 1..1131: mol_type = genomic DNA
atgacaaaattattttcaaattataaacgggcagcgattgattttgcttcggctcaaggcaattatttgacagataccactggcaagacctatttggatttttcatctggtatcggggtgactaatctttgctatcatccccgtgtcaatcaagctctaacagatcaggtaggcaagattttgcaccagcctaacctttattacaatcagttgcaagaagatgttgccggtcttttaactggtgacaaggactatctggcctttttctgtaacagtggtgcagaagccaatgaggcagccataaaaatcgcccgtaaggcatcgggtaaacaagaaatcattaccttccaaaattccttccacggtcgaaccttcggatccatgtctgccactggtcaggataaaatcaaacaaggttttggtgaaggcgtgccccactttagctatgccatttttaacgacatggacagtgttaaggctctagccaataaagaaacgtcggccattatgcttgagttggttcaaggagaatcagatgttcaaccagcggacaaggcctttgtcaaggctttgtctgattactgtcaggaaactggtatttatctcatcgtagacgaagttcagactggtattggtcgtactggcaagctctttgcttatgaacattacgatattgagccagacatctttaccctggccaaaggtttggcaaatggtgtaccagttggtgccatgcttgctaagtcatctcttggaggggctttctcttatggaagtcacggctctacttttggtggcaataaactatctatggcagcggccaaggctacgcttgaagtcatgttgactccaggttttctggatactgcccttgaaaatgggaacaagctacaagttaaattgcaagaggtcttgtctgataaagagacagtgaccactgtgcgtggattgggctacatgattggaattgagactactggaaatctgagtgaactggtacaagctgctagagataagggattaattgtcttaactgcagggacaaatgttatccgtcttctgcctccaatcactctaagtgatgctgaaatcgaaaaaggtgtggctatcctatcagaaatattttactag

SEQ ID NO: 176 - protein, 376 aa, Streptococcus thermophilus
MTKLFSNYKRAAIDFASAQGNYLTDTTGKTYLDFSSGIGVTNLCYHPRVNQALTDQVGKILHQPNLYYNQLQEDVAGLLTGDKDYLAFFCNSGAEANEAAIKIARKASGKQEIITFQNSFHGRTFGSMSATGQDKIKQGFGEGVPHFSYAIFNDMDSVKALANKETSAIMLELVQGESDVQPADKAFVKALSDYCQETGIYLIVDEVQTGIGRTGKLFAYEHYDIEPDIFTLAKGLANGVPVGAMLAKSSLGGAFSYGSHGSTFGGNKLSMAAAKATLEVMLTPGFLDTALENGNKLQVKLQEVLSDKETVTTVRGLGYMIGIETTGNLSELVQAARDKGLIVLTAGTNVIRLLPPITLSDAEIEKGVAILSEIFY

SEQ ID NO: 177 - DNA, 912 bp, Streptococcus thermophilus
  source 1..912: mol_type = genomic DNA
atgacttttgaaacgctcaccattgaaaatttaacaaaatcatttgataaacaacattttgctaataaccatatcttcttagagctagaagctgcaaaaatcacagctcttataggacataatgatgctggtaaatcaactcttttaaagcaaatgattggttttattaagcctgatcagggagatgttcactacggagatatttcttttatacataaccgtaaacaagctcgaacaatgatgagttatatgtcacaacagtatgcacctcttgaaaagttaactgttgagcaaaatttagagatgattggaagaatgagagggttaagtacctcagaactccaaaaagaaattgagcaccttctagcagatctggaaatagtagaataccgtgataaaaaagggaagaatctttcaggaggcttaaaacggcttacatcttttgccatggcattaattggctctgctgatatcatgcttcttgacgaaccaaccaacgatgttgatcctgtacgtcgtgaaaaacagtggcgattattacaaaaactggcttataaaggacacactattatcattgttacccacaatctactagaagttgaaaaatatgcggataattttgctctttttaaccatggtaaattattaaaatctggtccagtacaccaaatgtcttataaaaatcattatcaaatttctttcaattttgttagtaaagaatttttaagtctattcgaacattttacgatggtcaatggttgtgtttgtcagatggaaattgaagaaagtgaattaacccgaatactcattcaagttaaggaaggtatagaaaaagggcttatttcaaacctttcagttaagatgaaaacattatctatttccgatatttataaagaggagcttgttaactga

SEQ ID NO: 178 - protein, 303 aa, Streptococcus thermophilus
MTFETLTIENLTKSFDKQHFANNHIFLELEAAKITALIGHNDAGKSTLLKQMIGFIKPDQGDVHYGDISFIHNRKQARTMMSYMSQQYAPLEKLTVEQNLEMIGRMRGLSTSELQKEIEHLLADLEIVEYRDKKGKNLSGGLKRLTSFAMALIGSADIMLLDEPTNDVDPVRREKQWRLLQKLAYKGHTIIIVTHNLLEVEKYADNFALFNHGKLLKSGPVHQMSYKNHYQISFNFVSKEFLSLFEHFTMVNGCVCQMEIEESELTRILIQVKEGIEKGLISNLSVKMKTLSISDIYKEELVN

SEQ ID NO: 179 - DNA, 1146 bp, Streptococcus thermophilus
  source 1..1146: mol_type = genomic DNA
atgacagaagtattttacgatcatttagcaaagacgcgtcatcagattcaccaacatccagaggtttctggggaggagcatgagacgacagtctttttgaaagcttatttgaaaaatctaggtgttgagcctttaaattatcccttgaagacaggactcatcgctgagattgggtcaggtcatcctattattgccttgcgtgctgatattgatgccctaccgattaaggaaaagacaggacttccttatgctagcgacaatggtgccatgcatgcttatggtcatgattttcaccagacctctctgcttggtgctgctcagcttttaaaagaaagagaagcagagcttaaggggactgttcgattaatttttcaaccggctgaggaaaatttccaaggggcctatcaggtcattgaagcgggcggtattgagggtgtgtcagctatcatcggttatcacaataatcctcacttgaaacctggtcagattggccttcgttcaggtgctattttggcaggcgtggagcagtttagggttgatgtcaagggtgtttcgtcccatgcagcccgtcctgatttgggtgtggatacggtcttggttactacgactatcattaataatttacagcaaattgtggctcggacggtatctccctttgagtcagctgtattgtcagttactcatatcgaagttggaaatacctggaatgttcttccagcagccggcttcttcgaaggaacgattcggactttcaatcctaaggttcgggaagatgtcattgctcgctttgaaaaagttgttcaggcgacagcggatcagtttggtgctcaagttgacattacttggggtaattcaccttacgtgacctacaatgaccagacactcacaccactcatctttgaaaattctaaagccttcgcagaagtgattgaaacgctgccatcaacaggcggagaagactttgcggcttatcaaaaggaaataccaggagtctttgcctttgtcggcactaatggtgaggaggatgccccaggctggcatcatgatgatttccttgtgaaagatgaagccttgcctgtagccgttaattactatgtggaaaacgctcttttcctccttgattattttaaggagcaagacgactga

SEQ ID NO: 180 - protein, 381 aa, Streptococcus thermophilus
MTEVFYDHLAKTRHQIHQHPEVSGEEHETTVFLKAYLKNLGVEPLNYPLKTGLIAEIGSGHPIIALRADIDALPIKEKTGLPYASDNGAMHAYGHDFHQTSLLGAAQLLKEREAELKGTVRLIFQPAEENFQGAYQVIEAGGIEGVSAIIGYHNNPHLKPGQIGLRSGAILAGVEQFRVDVKGVSSHAARPDLGVDTVLVTTTIINNLQQIVARTVSPFESAVLSVTHIEVGNTWNVLPAAGFFEGTIRTFNPKVREDVIARFEKVVQATADQFGAQVDITWGNSPYVTYNDQTLTPLIFENSKAFAEVIETLPSTGGEDFAAYQKEIPGVFAFVGTNGEEDAPGWHHDDFLVKDEALPVAVNYYVENALFLLDYFKEQDD

SEQ ID NO: 181 - DNA, 2556 bp, Streptococcus thermophilus
  source 1..2556: mol_type = genomic DNA
atgaagcgacttatagagagacttcaacctttaaaaactgccgttaaatctgtcttctttatttctattgcggttcttgttgttgtagagttgattcgtttaaaacgaacgatcactttagaatcgctggagtcagctttatcaggtctgtcaatttggcatcttgctttgatggtagtgataggcttagttgctgtgttacccatgctcttttatgaccttatcttgaataaggagctagagacggattattccaaatcctatatactagaaaagagttgggctgtcaataccattaataacttgtctggttttgccggtttagttgacgtgggattgaggtattccttttattctgaagatgggcatgaaaagtctggtgttaaggcactatcacgtttattgccttacttcatgtcgggcttgtcgctcctaagtggcttggtgtttgctaccttttggtttttccctctaagtcctgacttgagaaaatattggttacttttactaggaatctttttatacctaccttttattttctttgtctcaagtcgcgaaaaattgccttattttgggcaagtccctttgaaaacgagactaagccttattttagtatcgactgctgaatggggcacagctcttgtgagttttctgagtgtggcttatttaatgggtctacatgttcctctttataatctgattcccctatattttctagcggttatcattggtatcttttccatgattccaggtgggattggtagttttgatttgattgtgattagtggtctgacgtctatgggtgtcagcaatgctcttgcggtcagtctactattgctctttcgtctaacctactacatcattccatttttattaggtgttgtcttctttttcaaacacatgggaggtcacatcaatgataaatacttcaatattccttcacgagtatttggtggaagtttccatcttatccttgtttatttgttacgttttttaggaatcttcttgattttatctgccctaatcccagaaagcttggcaaatgtttactttattagccgtttcgatcctattcaggaacagttgatctggcagtttccaagtattttatttggaaccctctttgttttcatgtctcgcctcatcaggcgacgcgttaagggagcttttattgtatccttgctgaccttcgtcttgactttgatttacgtcaatctcaatggtacttcctggaccatgtctttcatgattgtgctttctatgatacttatgttacttataaggaaaagattgaatcgtcactattttgtttatagctgggaagacaaaaccaaggatttcctttttttagcctttaccattttggtcctccttgttctgggtggtagtggtttttggtctcatttgttaccacctaggaaccgagatattcttggacattttgttcatatttggtttgatattttactggcatcggtaatcattgcaaccattgtttggcttgtactgagagttttggcacctcgtaagcattttggtcaaagcatggataatgagcgttttacagcacttcttgagaaatatgggggcgcatcagaaagtgctctagcttatttacatgataaacgcctcttttggtatcgtgtcgatggacaagatcaggtagtttttcaatttgcacaaatcagtaacaagtgtgtgattatgggcaatcctattggtaatgaaaaatattatcgtccggcttgggaagctttcttgaaaaacttatctgattggaatttacaggctttattttatgaggcggatgagagagtgacccttatgcttcatgattatggctttgacttcatgaaatttggagaaaatgccatggttgatttgacgacattttctgttgatggtaagcatggtaagaagttccgtaaaccaaccaatcgtgtggaaaaagcaggcttccagtttaaattacttaatcctccattttcggagactcagatgcaggaaatgaaggcagtctcagatatatggcttaacggtcgtaaggagaaaggtttctctcttggtttctttgatgaagcctatttacaacaagcaccgattgctatcgttgagagtaaagaaggagaaattgtggccttcgctaatatcatgccaacccaaaataaacgggtggcaaccattgatcttatgcgttatgactttaaaaaggctcctgaaggcatcatggactacctttttgtcaaactcttccaatacttcaaagaagaaggcaagcaatactttgatatggggatggctcccctagctaacgtaggtactgaagaagacagtttcatagaggaaaaggttgccaatttagtttatgtttttgctcagcgtttttactcttttagtggattgcaacgttacaaagaaaaattctctccaatttggtctccaaaatacattgtctatccaaaaagaacctggcttttgtttgatatgattgctattttcaggattgataatcgtaaaattgaagataaaatgaagaaaagacgtctttggaagtaa

SEQ ID NO: 182 - protein, 851 aa, Streptococcus thermophilus
MKRLIERLQPLKTAVKSVFFISIAVLVVVELIRLKRTITLESLESALSGLSIWHLALMVVIGLVAVLPMLFYDLILNKELETDYSKSYILEKSWAVNTINNLSGFAGLVDVGLRYSFYSEDGHEKSGVKALSRLLPYFMSGLSLLSGLVFATFWFFPLSPDLRKYWLLLLGIFLYLPFIFFVSSREKLPYFGQVPLKTRLSLILVSTAEWGTALVSFLSVAYLMGLHVPLYNLIPLYFLAVIIGIFSMIPGGIGSFDLIVISGLTSMGVSNALAVSLLLLFRLTYYIIPFLLGVVFFFKHMGGHINDKYFNIPSRVFGGSFHLILVYLLRFLGIFLILSALIPESLANVYFISRFDPIQEQLIWQFPSILFGTLFVFMSRLIRRRVKGAFIVSLLTFVLTLIYVNLNGTSWTMSFMIVLSMILMLLIRKRLNRHYFVYSWEDKTKDFLFLAFTILVLLVLGGSGFWSHLLPPRNRDILGHFVHIWFDILLASVIIATIVWLVLRVLAPRKHFGQSMDNERFTALLEKYGGASESALAYLHDKRLFWYRVDGQDQVVFQFAQISNKCVIMGNPIGNEKYYRPAWEAFLKNLSDWNLQALFYEADERVTLMLHDYGFDFMKFGENAMVDLTTFSVDGKHGKKFRKPTNRVEKAGFQFKLLNPPFSETQMQEMKAVSDIWLNGRKEKGFSLGFFDEAYLQQAPIAIVESKEGEIVAFANIMPTQNKRVATIDLMRYDFKKAPEGIMDYLFVKLFQYFKEEGKQYFDMGMAPLANVGTEEDSFIEEKVANLVYVFAQRFYSFSGLQRYKEKFSPIWSPKYIVYPKRTWLLFDMIAIFRIDNRKIEDKMKKRRLWK